(19) United States
(12) Patent Application Publication
(10) Pub. No.: US 2023/0407402 A1
(43) Pub. Date: Dec. 21, 2023

(54) PREDICTION OF A RESPONSE OF A PROSTATE CANCER SUBJECT TO THERAPY OR PERSONALIZATION OF THERAPY OF A PROSTATE CANCER SUBJECT

(71) Applicant: KONINKLIJKE PHILIPS N.V., EINDHOVEN (NL)

(72) Inventors: Ralf Dieter Hoffmann, Brueggen (DE); Monique Stoffels, Eindhoven (NL)

(21) Appl. No.: 18/023,598

(22) PCT Filed: Aug. 24, 2021
(86) PCT No.: PCT/EP2021/073342

§ 371 Date: Feb. 27, 2023

(30) Foreign Application Priority Data
Aug. 31, 2020 (EP) 20193650.7

(51) Int. Cl. C12Q 1/6886

(52) U.S. Cl. CPC C12Q 1/6886; C12Q 2600/158; C12Q 2600/106

(57) ABSTRACT

The invention relates to a method of predicting a response of a prostate cancer subject to therapy or of personalizing therapy of a prostate cancer subject, comprising determining or receiving the result of a determination of a first gene expression profile for each of one or more immune defense response genes, of a second gene expression profile for each of one or more T-Cell receptor signaling genes, and of a third gene expression profile for each of one or more PDE4D7 correlated genes, said first, second, and third expression profile(s) being determined in a biological sample obtained from the subject, determining the prediction of the therapy response or the personalization of the therapy based on the first, second, and third gene expression profile(s), and, optionally, providing the prediction or the personalization or a therapy recommendation based on the prediction or the personalization to a medical caregiver or the subject.

### FIELD OF THE INVENTION

The invention relates to a method of predicting a response of a prostate cancer subject to therapy or of personalizing therapy of a prostate cancer subject, and to an apparatus for predicting a response of a prostate cancer subject to therapy or for personalizing therapy of a prostate cancer subject. Moreover, the invention relates to a diagnostic kit, to a use of the kit, to a use of the kit in a method of predicting a response of a prostate cancer subject to therapy or of personalizing therapy of a prostate cancer subject, to a use of first, second, and third gene expression profile(s) in a method of predicting a response of a prostate cancer subject to therapy or of personalizing therapy of a prostate cancer subject, and to a corresponding computer program product.

### BACKGROUND OF THE INVENTION

Cancer is a class of diseases in which a group of cells displays uncontrolled growth, invasion and sometimes metastasis. These three malignant properties of cancers differentiate them from benign tumours, which are self-limited and do not invade or metastasize. Prostate Cancer (PCa) is the second most commonly-occurring non-skin malignancy in men, with an estimated 1.3 million new cases diagnosed and 360,000 deaths world-wide in 2018 (see Bray F. et al., “Global cancer statistics 2018: GLOBOCAN estimates of incidence and mortality worldwide for 36 cancers in 185 countries”, CA Cancer J Clin, Vol. 68, No. 6, pages 394-424, 2018). In the US, about 90% of the new cases concern localized cancer, meaning that metastases have not yet been formed (see ACS (American Cancer Society), “Cancer Facts & FIGURES 2010”, 2010).

For the treatment of primary localized prostate cancer, several radical therapies are available, of which surgery (radical prostatectomy, RP) and radiation therapy (RT) are most commonly used. RT is administered via an external beam or via the implantation of radioactive seeds into the prostate (brachytherapy) or a combination of both. It is especially preferable for patients who are not eligible for surgery or have been diagnosed with a tumour in an advanced localized or regional stage. Radical RT is provided to up to 50% of patients diagnosed with localized prostate cancer in the US (see ACS, 2010, ibid).

After treatment, prostate cancer antigen (PSA) levels in the blood are measured for disease monitoring. An increase of the blood PSA level provides a biochemical surrogate measure for cancer recurrence or progression. However, the variation in reported biochemical progression-free survival (bPFS) is large (see Grimm P. et al., “Comparative analysis of prostate-specific antigen free survival outcomes for patients with low, intermediate and high risk prostate cancer treatment by radical therapy. Results from the Prostate Cancer Results Study Group”, BJU Int, Suppl. 1, pages 22-29, 2012). For many patients, the bPFS at 5 or even 10 years after radical RT may lie above 90%. Unfortunately, for the group of patients at medium and especially higher risk of recurrence, the bPFS can drop to around 40% at 5 years, depending on the type of RT used (see Grimm P. et al., 2012, ibid).

A large number of the patients with primary localized prostate cancer that are not treated with RT will undergo RP (see ACS, 2010, ibid). After RP, an average of 60% of patients in the highest risk group experience biochemical recurrence after 5 and 10 years (see Grimm P. et al., 2012, ibid). In case of biochemical progression after RP, one of the main challenges is the uncertainty whether this is due to recurring localized disease, one or more metastases or even an indolent disease that will not lead to clinical disease progression (see Dal Pra A. et al., “Contemporary role of postoperative radiotherapy for prostate cancer”, Transl Androl Urol, Vo. 7, No. 3, pages 399-413, 2018, and Herrera F. G. and Berthold D. R., “Radiation therapy after radical prostatectomy: Implications for clinicians”, Front Oncol, Vol. 6, No. 117, 2016). RT to eradicate remaining cancer cells in the prostate bed is one of the main treatment options to salvage survival after a PSA increase following RP. The effectiveness of salvage radiotherapy (SRT) results in 5-year bPFS for 18% to 90% of patients, depending on multiple factors (see Herrera F. G. and Berthold D. R., 2016, ibid, and Pisansky T. M. et al., “Salvage radiation therapy dose response for biochemical failure of prostate cancer after prostatectomy—A multi-institutional observational study”, Int J Radiat Oncol Biol Phys, Vol. 96, No. 5, pages 1046-1053, 2016).

It is clear that for certain patient groups, radical or salvage RT is not effective. Their situation is even worsened by the serious side effects that RT can cause, such as bowel inflammation and dysfunction, urinary incontinence and erectile dysfunction (see Resnick M. J. et al., “Long-term functional outcomes after treatment for localized prostate cancer”, N Engl J Med, Vol. 368, No. 5, pages 436-445, 2013, and Hegarty S. E. et al., “Radiation therapy after radical prostatectomy for prostate cancer: Evaluation of complications and influence of radiation timing on outcomes in a large, population-based cohort”, PLoS One, Vol. 10, No. 2, 2015). In addition, the median cost of one course of RT based on Medicare reimbursement is $18,000, with a wide variation up to about $40,000 (see Paravati A. J. et al., “Variation in the cost of radiation therapy among medicare patients with cancer”, J Oncol Pract, Vol. 11, No. 5, pages 403-409, 2015). These figures do not include the considerable longitudinal costs of follow-up care after radical and salvage RT.

An improved prediction of effectiveness of RT for each patient, be it in the radical or the salvage setting, would improve therapy selection and potentially survival. This can be achieved by 1) optimizing RT for those patients where RT is predicted to be effective (e.g., by dose escalation or a different starting time) and 2) guiding patients where RT is predicted not to be effective to an alternative, potentially more effective form of treatment. Further, this would reduce suffering for those patients who would be spared ineffective therapy and would reduce costs spent on ineffective therapies.

Numerous investigations have been conducted into measures for response prediction of radical RT (see Hall W. A. et al., “Biomarkers of outcome in patients with localized prostate cancer treated with radiotherapy”, Semin Radiat Oncol, Vol. 27, pages 11-20, 2016, and Raymond E. et al., “An appraisal of analytical tools used in predicting clinical outcomes following radiation therapy treatment of men with prostate cancer: A systematic review”, Radiat Oncol, Vol. 12, No. 1, page 56, 2017) and SRT (see Herrera F. G. and Berthold D. R., 2016, ibid). Many of these measures depend on the concentration of the blood-based biomarker PSA. Metrics investigated for prediction of response before start of RT (radical as well as salvage) include the absolute value of the PSA concentration, its absolute value relative to the prostate volume, the absolute increase over a certain time and the doubling time. Other frequently considered factors are the Gleason score and the clinical tumour stage. For the SRT setting, additional factors are relevant, e.g., surgical margin status, time to recurrence after RP, pre-/peri-surgical PSA values and clinico-pathological parameters.

Although these clinical variables provide limited improvements in patient stratification in various risk groups, there is a need for better predictive tools.

A wide range of biomarker candidates in tissue and bodily fluids has been investigated, but validation is often limited and generally demonstrates prognostic information and not a predictive (therapy-specific) value (see Hall W. A. et al., 2016, ibid). A small number of gene expression panels is currently being validated by commercial organizations. One or a few of these may show predictive value for RT in future (see Dal Pra A. et al., 2018, ibid).

In conclusion, a strong need for better prediction of response to RT as well as for personalization of therapy remains, for primary prostate cancer as well as for the post-surgery setting. The same holds true for the prediction of response to salvage androgen deprivation therapy (SADT) and cytotoxic chemotherapy (CTX).

### SUMMARY OF THE INVENTION

It is an objective of the invention to provide a method of predicting a response of a prostate cancer subject to therapy or of personalizing therapy of a prostate cancer subject, and an apparatus for predicting a response of a prostate cancer subject to therapy or for personalizing therapy of a prostate cancer subject, which allow to make better treatment decisions. It is a further aspect of the invention to provide a diagnostic kit, a use of the kit, a use of the kit in a method of predicting a response of a prostate cancer subject to therapy or of personalizing therapy of a prostate cancer subject, a use of first, second, and third gene expression profile(s) in a method of predicting a response of a prostate cancer subject to therapy or of personalizing therapy of a prostate cancer subject, and a corresponding computer program product.

In a first aspect of the present invention, a method of predicting a response of a prostate cancer subject to therapy or of personalizing therapy of a prostate cancer subject is presented, comprising:
determining or receiving the result of a determination of a first gene expression profile for each of one or more, for example, 1, 2, 3, 4, 5, 6, 7, 8, 9, 10, 11, 12, 13 or all, immune defense response genes selected from the group consisting of: AIM2, APOBEC3A, CIAO1, DDX58, DHX9, IFI16, IFIH1, IFIT1, IFIT3, LRRFIP1, MYD88, OAS1, TLR8, and ZBP1, said first gene expression profile(s) being determined in a biological sample obtained from the subject,
determining or receiving the result of a determination of a second gene expression profile for each of one or more, for example, 1, 2, 3, 4, 5, 6, 7, 8, 9, 10, 11, 12, 13, 14, 15, 16 or all, T-Cell receptor signaling genes selected from the group consisting of: CD2, CD247, CD28, CD3E, CD3G, CD4, CSK, EZR, FYN, LAT, LCK, PAG1, PDE4D, PRKACA, PRKACB, PTPRC, and ZAP70, said second gene expression profile(s) being determined in a biological sample obtained from the subject,
determining or receiving the result of a determination of a third gene expression profile for each of one or more, for example, 1, 2, 3, 4, 5, 6, 7 or all, PDE4D7 correlated genes selected from the group consisting of: ABCC5, CUX2, KIAA1549, PDE4D, RAP1GAP2, SLC39A11, TDRD1, and VWA2, said third gene expression profile(s) being determined in a biological sample obtained from the subject,
determining the prediction of the therapy response or the personalization of the therapy based on the first, second, and third gene expression profile(s), and
optionally, providing the prediction or the personalization or a therapy recommendation based on the prediction or the personalization to a medical caregiver or the subject.

Therefore, in an embodiment the invention relates to a method of predicting a response of a prostate cancer subject to therapy or of personalizing therapy of a prostate cancer subject is presented, comprising:
determining of a first gene expression profile for each of one or more, for example, 1, 2, 3, 4, 5, 6, 7, 8, 9, 10, 11, 12, 13 or all, immune defense response genes selected from the group consisting of: AIM2, APOBEC3A, CIAO1, DDX58, DHX9, IFI16, IFIH1, IFIT1, IFIT3, LRRFIP1, MYD88, OAS1, TLR8, and ZBP1, said first gene expression profile(s) being determined in a biological sample obtained from the subject,
determining of a second gene expression profile for each of one or more, for example, 1, 2, 3, 4, 5, 6, 7, 8, 9, 10, 11, 12, 13, 14, 15, 16 or all, T-Cell receptor signaling genes selected from the group consisting of: CD2, CD247, CD28, CD3E, CD3G, CD4, CSK, EZR, FYN, LAT, LCK, PAG1, PDE4D, PRKACA, PRKACB, PTPRC, and ZAP70, said second gene expression profile(s) being determined in a biological sample obtained from the subject,
determining of a third gene expression profile for each of one or more, for example, 1, 2, 3, 4, 5, 6, 7 or all, PDE4D7 correlated genes selected from the group consisting of: ABCC5, CUX2, KIAA1549, PDE4D, RAP1GAP2, SLC39A11, TDRD1, and VWA2, said third gene expression profile(s) being determined in a biological sample obtained from the subject,
determining the prediction of the therapy response or the personalization of the therapy based on the first, second, and third gene expression profile(s), and
optionally, providing the prediction or the personalization or a therapy recommendation based on the prediction or the personalization to a medical caregiver or the subject. For example the expression levels may be determined using a reporter gene, Northern blotting, western blotting, fluorescent in situ hybridiczation (FISH), reverse transcription polymerase chain reaction (RT-PCT), quantitative polymerase chain reaction (qPCR), serial analysis of gene expression (SAGE), DNA microarray, RNA sequencing or Tiling array.

In an alternative embodiment the invention relates to a computer implemented method of predicting a response of a prostate cancer subject to therapy or of personalizing therapy of a prostate cancer subject is presented, comprising:
receiving the result of a determination of a first gene expression profile for each of one or more, for example, 1, 2, 3, 4, 5, 6, 7, 8, 9, 10, 11, 12, 13 or all, immune defense response genes selected from the group consisting of: AIM2, APOBEC3A, CIAO1, DDX58, DHX9, IFI16, IFIH1, IFIT1, IFIT3, LRRFIP1, MYD88, OAS1, TLR8, and ZBP1, said first gene expression profile(s) being determined in a biological sample obtained from the subject,
receiving the result of a determination of a second gene expression profile for each of one or more, for example, 1, 2, 3, 4, 5, 6, 7, 8, 9, 10, 11, 12, 13, 14, 15, 16 or all, T-Cell receptor signaling genes selected from the group consisting of: CD2, CD247, CD28, CD3E, CD3G, CD4, CSK, EZR, FYN, LAT, LCK, PAG1, PDE4D, PRKACA, PRKACB, PTPRC, and ZAP70, said second gene expression profile(s) being determined in a biological sample obtained from the subject,
receiving the result of a determination of a third gene expression profile for each of one or more, for example, 1, 2, 3, 4, 5, 6, 7 or all, PDE4D7 correlated genes selected from the group consisting of: ABCC5, CUX2, KIAA1549, PDE4D, RAP1GAP2, SLC39A11, TDRD1, and VWA2, said third gene expression profile(s) being determined in a biological sample obtained from the subject,
determining the prediction of the therapy response or the personalization of the therapy based on the first, second, and third gene expression profile(s), and
optionally, providing the prediction or the personalization or a therapy recommendation based on the prediction or the personalization to a medical caregiver or the subject.

In recent years, the importance of the immune system in cancer inhibition as well as in cancer initiation, promotion and metastasis has become very evident (see Mantovani A. et al., “Cancer-related inflammation”, Nature, Vol. 454, No. 7203, pages 436-444, 2008, and Giraldo N. A. et al., “The clinical role of the TME in solid cancer”, Br J Cancer, Vol. 120, No. 1, pages 45-53, 2019). The immune cells and the molecules they secrete form a crucial part of the tumour microenvironment and most immune cells can infiltrate the tumour tissue. The immune system and the tumour affect and shape one another. Thus, anti-tumour immunity can prevent tumour formation while an inflammatory tumour environment may promote cancer initiation and proliferation. At the same time, tumour cells that may have originated in an immune system-independent manner will shape the immune microenvironment by recruiting immune cells and can have a pro-inflammatory effect while also suppressing anti-cancer immunity.

Some of the immune cells in the tumour microenvironment will have either a general tumour-promoting or a general tumour-inhibiting effect, while other immune cells exhibit plasticity and show both tumour-promoting and tumour-inhibiting potential. Thus, the overall immune microenvironment of the tumour is a mixture of the various immune cells present, the cytokines they produce and their interactions with tumour cells and with other cells in the tumour microenvironment (see Giraldo N. A. et al., 2019, ibid).

The principles described above with regard to the role of the immune system in cancer in general also apply to prostate cancer. Chronic inflammation has been linked to the formation of benign as well as malignant prostate tissue (see Hall W. A. et al., 2016, ibid) and most prostate cancer tissue samples show immune cell infiltrates. The presence of specific immune cells with a pro-tumour effect has been correlated with worse prognosis, while tumours in which natural killer cells were more activated showed better response to therapy and longer recurrence-free periods (see Shiao S. L. et al., “Regulation of prostate cancer progression by tumor microenvironment”, Cancer Lett, Vol. 380, No. 1, pages 340-348, 2016).

While a therapy will be influenced by the immune components of the tumour microenvironment, RT itself extensively affects the make-up of these components (see Barker H. E. et al., “The tumor microenvironment after radiotherapy: Mechanisms of resistance or recurrence”, Nat Rev Cancer, Vol. 15, No. 7, pages 409-425, 2015). Because suppressive cell types are comparably radiation-insensitive, their relative numbers will increase. Counteractively, the inflicted radiation damage activates cell survival pathways and stimulates the immune system, triggering inflammatory responses and immune cell recruitment. Whether the net effect will be tumour-promoting or tumour-suppressing is as yet uncertain, but its potential for enhancement of cancer immunotherapies is being investigated.

The present invention is based on the idea that, since the status of the immune system and of the immune microenvironment have an impact on therapy effectiveness, the ability to identify markers predictive for this effect might help to be better able to predict the response to pre-surgical RT as well as post-surgical therapies like SRT, SADT or CTX.

The present invention is further based on the idea that, since the known PDE4D7 biomarker has been proven to be a good predictor of radiotherapy response, the ability to identify markers that are highly correlated with the PDE47 biomarker might also help to be better able to predict response to pre-surgical RT as well as post-surgical therapies like SRT, SADT or CTX.

### Immune Response Defense Genes

The integrity and stability of genomics DNA is permanently under stress induced by various cell internal and external factors like exposure to radiation, viral or bacterial infections, but also oxidation and replication stress (see Gasser S. et al., “Sensing of dangerous DNA”, Mechanisms of Aging and Development, Vol. 165, pages 33-46, 2017). In order to maintain DNA structure and stability, a cell must be able to recognize all types of DNA damages like single or double strand breaks etc. induced by various factors. This process involves the participation of a multitude of specific proteins depending on the kind of damage as part of DNA recognition pathways.

Recent evidence suggests that mis-localized DNA (e.g., DNA unnaturally appearing in the cytosolic fraction of the cell in contrast to the nucleus) and damaged DNA (e.g., through mutations occurring in cancer development) is used by the immune system to identify infected or otherwise diseased cells while genomic and mitochondrial DNA present in healthy cells is ignored by DNA recognition pathways. In diseased cells, cytosolic DNA sensor proteins have been demonstrated to be involved in the detection of DNA occurring unnaturally in the cytosol of the cell. Detection of such DNA by different nucleic acid sensors translates into similar responses leading to nuclear factor kappa-B (NF-kB) and interferon type I (IFN type I) signalling followed by the activation of innate immune system components. While the recognition of viral DNA is known to induce an IFN type I response, evidence that sensing of DNA damage can initiate immune responses has only recently been accumulating.

TLR9 (Toll-like receptor 9) located in the endosomes was one of the first DNA sensors molecules identified to be involved in the immune recognition of DNA by signalling downstream via the adaptor protein myeloid differentiation primary-response protein88 (MYD88). This interaction in turn activates mitogen-activated protein kinases (MAPKs) and NF-kB. TLR9 also induces the generation of type I interferons through the activation of IRF7 via IkB Kinase alpha (IKKalpha) in plasmacytoid dendritic cells (pDCs). Various other DNA immune receptors including IFI16 (IFN-gamma-inducible protein 16), cGAS (cyclic DMP-AMP synthase, DDX41 (DEAD-box helicase 41), as well as ZBP1 (Z-DNA-binding protein 1) interact with STING (stimulator of IFN genes), which activates the IKK complex and IRF3 through TBK1 (TANK binding kinase 1). ZBP1 also activates NF-kB via recruitment of RIP1 and RIP3 (receptor-interacting protein 1 and 3, respectively). While the helicase DHX36 (DEAH-box helicase 36) interacts in a complex with TRID to induce NF-kB and IRF-3/7 the DHX9 helicase stimulates MYD88-dependent signalling in plasmacytoid dendritic cells. The DNA sensor LRRFIP1 (leucine-rich repeat flightless-interacting protein) complexes with beta-catenin to activate the transcription of IRF3 whereas AIM2 (absent in melanoma 2) recruits the adaptor protein ASC (apoptosis speck-like protein) to induce a caspase-1-activating inflammasome complex leading to the secretion of interleukin-lbeta (IL-1beta) and IL-18 (see FIG. 1 of Gasser S. et al., 2017, ibid, which provides a schematic overview of DNA damage and DNA sensor pathways leading to the production of inflammatory cytokines and the expression of ligands for activating innate immune receptors. Members of the non-homologous end joining pathway (orange), homologous recombination (red), inflammasome (dark green), NF-kB and interferon responses (light green) are shown).

The factors and mechanisms responsible for activating the DNA sensor pathways in cancer are currently not well elucidated. It will be important to identify the intratumoral DNA species, sensors and pathways implicated in the expression of IFNs in different cancer types at all stages of the disease. In addition to therapeutic targets in cancer, such factors may also have prognostic and predictive value. Novel DNA sensor pathway agonists and antagonists are currently being developed and tested in preclinical trials. Such compounds will be useful in characterizing the role of DNA sensor pathways in the pathogenesis of cancer, autoimmunity and potentially other diseases.

The identified immune defense response genes ZBP1, and AIM2, APOBEC3A, CIAO1, DDX58, DHX9, IFI16, IFIH1, IFIT1, IFIT3, LRRFIP1, MYD88, OAS1, TLR8, and ZBP1, respectively, were identified as follows: A group of 538 prostate cancer patients were treated with RP and the prostate cancer tissue was stored together with clinical (e.g., pathological Gleason grade group (pGGG), pathology state (pT stage)) as well as relevant outcome parameters (e.g., biochemical recurrence (BCR), metastatic recurrence, prostate cancer specific death (PCa death), salvage radiation treatment (SRT), salvage androgen deprivation treatment (SADT), chemotherapy (CTX)). For each of these patients, a PDE4D7 score was calculated and categorized into four PDE4D7 score classes as described in Alves de Inda M. et al., “Validation of Cyclic Adenosine Monophosphate Phosphodiesterase-4D7 for its Independent Contribution to Risk Stratification in a Prostate Cancer Patient Cohort with Longitudinal Biological Outcomes”, Eur Urol Focus, Vol. 4, No. 3, pages 376-384, 2018. PDE4D7 score class 1 represents patient samples with lowest expression levels of PDE4D7, whereas PDE4D7 score class 4 represents patient samples with highest levels of PDE4D7 expression. RNASeq expression data (TPM—Transcripts Per Million) of the 538 prostate cancer subjects was then investigated for differential gene expression between the PDE4D7 score classes 1 and 4. In particular, it was determined for around 20,000 protein coding transcripts whether the mean expression level of the PDE4D7 score class 1 patients was more than twice as high as the mean expression level of the PDE4D7 score class 4 patients. This analysis resulted in 637 genes with a ratio PDE4D7 score class 1/PDE4D7 score class 4 of >2 with a minimum mean expression of 1 TPM in each of the four PDE4D7 score classes. These 637 genes were then further subjected to molecular pathway analysis (www.david.ncifcrf.gov), which resulted in a range of enriched annotation clusters. The annotation cluster #2 demonstrated enrichment (enrichment score: 10.8) in 30 genes with a function in defense response to viruses, negative regulation of viral genome replication as well as type I interferon signaling. A further heat map analysis confirmed that these immune defense response genes were generally higher expressed in samples from patients in PDE4D7 score class 1 than from patients in PDE4D7 score class 4. The class of genes with a function in defense response to viruses, negative regulation of viral genome replication as well as type I interferon signaling was further enriched to 61 genes by literature search to identify additional genes with the same molecular function. A further selection from the 61 genes was made based on the combinatorial power to separate patients who died from prostate cancer vs. those who did not, resulting of a preferred set of 14 genes. It was found that the number of events (metastases, prostate cancer specific death) was enriched in sub-cohorts with a low expression of these genes compared to the total patient cohort (#538) and a sub-cohort of 151 patients undergoing salvage RT (SRT) after post-surgical disease recurrence.

### T-Cell Receptor Signaling Genes

An immune response against pathogens can be elicited at different levels: there are physical barriers, such as the skin, to keep invaders out. If breached, innate immunity comes into play; a first and fast non-specific response. If this is not sufficient, the adaptive immune response is elicited. This is much more specific and needs time to develop when encountering a pathogen for the first time. Lymphocytes are activated by interacting with activated antigen presenting cells from the innate immune system, and are also responsible for maintenance of memory for faster responses upon next encounters with the same pathogen.

As lymphocytes are highly specific and effective when activated, they are subject to negative selection for their ability to recognize self, a process known as central tolerance. As not all self-antigens are expressed at selection sites, peripheral tolerance mechanisms evolved as well, such as ligation of the TCR in absence of co-stimulation, expression of inhibitory co-receptors, and suppression by Tregs. A disturbed balance between activation and suppression may lead to autoimmune disorders, or immune deficiencies and cancer, respectively.

T-cell activation can have different functional consequences, depending on the location the type of T-cell involved. CD8+ T-cells differentiate into cytotoxic effector cells, whereas CD4+ T-cells can differentiate into Th1 (IFNγ secretion and promotion of cell mediated immunity) or Th2 (IL4/5/13 secretion and promotion of B cell and humoral immunity). Differentiation towards other, more recently identified T-cell subsets is also possible, for example the Tregs, which have a suppressive effect on immune activation (see Mosenden R. and Tasken K., “Cyclic AMP-mediated immune regulation—Overview of mechanisms of action in T-cells”, Cell Signal, Vol. 23, No. 6, pages 1009-1016, 2011, in particular, FIG. 4, which T-cell activation and its modulation by PKA, and Tasken K. and Ruppelt A., “Negative regulation of T-cell receptor activation by the cAMP-PKA-Csk signaling pathway in T-cell lipid rafts”, Front Biosci, Vol. 11, pages 2929-2939, 2006).

Both PKA and PDE4 regulated signaling intersect with TCR induced T-cell activation to fine-tune its regulation, with opposing effects (see Abrahamsen H. et al., “TCR- and CD28-mediated recruitment of phosphodiesterase 4 to lipid rafts potentiates TCR signaling”, J Immunol, Vol. 173, pages 4847-4848, 2004, in particular, FIG. 6, which shows opposing effects of PKA and PDE4 on TCR activation). The molecule that connects these effectors is cyclic AMP (cAMP), an intracellular second messenger of extracellular ligand action. In T-cells, it mediates effects of prostaglandins, adenosine, histamine, beta-adrenergic agonists, neuropeptide hormones and beta-endorphin. Binding of these extracellular molecules to GPCRs leads to their conformational change, release of stimulatory subunits and subsequent activation of adenylate cyclases (AC), which hydrolyze ATP to cAMP (see FIG. 6 of Abrahamsen H. et al., 2004, ibid). Although not the only one, PKA is the principal effector of cAMP signaling (see Mosenden R. and Tasken K., 2011, ibid, and Tasken K. and Ruppelt A., 2006, ibid). At a functional level, increased levels of cAMP lead to reduced IFNγ and IL-2 production in T-cells (see Abrahamsen H. et al., 2004, ibid). Aside from interfering with TCR activation, PKA has many more effector (see FIG. 15 of Torheim E. A., “Immunity Leashed-Mechanisms of Regulation in the Human Immune System”, Thesis for the degree of Philosophiae Doctor (PhD), The Biotechnology Centre of Ola, University of Oslo, Norway, 2009).

In naïve T-cells, hyperphosphorylated PAG targets Csk to lipid rafts. Via the Ezrin-EBP50-PAG scaffold complex PKA is targeted to Csk. Through specific phosphorylation by PKA, Csk can negatively regulate Lck and Fyn to dampen their activity and downregulate T-cell activation (see FIG. 6 of Abrahamsen H. et al., 2004, ibid). Upon TCR activation, PAG is dephosphorylated and Csk is released from the rafts. Dissociation of Csk is needed for T-cell activation to proceed. Within the same time course, a Csk-G3BP complex is formed and seems to sequester Csk outside lipid rafts (see Mosenden R. and Tasken K., 2011, ibid, and Tasken K. and Ruppelt A., 2006, ibid).

In contrast, combined TCR and CD28 stimulation mediates recruitment of the cyclic nucleotide phosphodiesterase PDE4 to lipid rafts, which enhances cAMP degradation (see FIG. 6 of Abrahamsen H. et al., 2004, ibid). As such, TCR induced production of cAMP is countered, and the T-cell immune response potentiated. Upon TCR stimulation alone, PDE4 recruitment may be too low to fully reduce the cAMP levels and therefore maximal T-cell activation cannot occur (see Abrahamsen H. et al., 2004, ibid).

Thus, by active suppression of proximal TCR signaling, signaling via cAMP-PKA-Csk is thought to set the threshold (cut-off) for T-cell activation. Recruitment of PDEs can counter this suppression. Tissue or cell-type specific regulation is accomplished through expression of multiple isoforms of AC, PKA, and PDEs. As mentioned above, the balance between activation and suppression needs to be tightly regulated to prevent development of autoimmune disorders, immune deficiencies and cancer.

The identified T-Cell receptor signaling genes CD2, CD247, CD28, CD3E, CD3G, CD4, CSK, EZR, FYN, LAT, LCK, PAG1, PDE4D, PRKACA, PRKACB, PTPRC, and ZAP70 were identified as follows: A group of 538 prostate cancer patients were treated with RP and the prostate cancer tissue was stored together with clinical (e.g., pathological Gleason grade group (pGGG), pathology state (pT stage)) as well as relevant outcome parameters (e.g., biochemical recurrence (BCR), metastatic recurrence, prostate cancer specific death (PCa death), salvage radiation treatment (SRT), salvage androgen deprivation treatment (SADT), chemotherapy (CTX)). For each of these patients, a PDE4D7 score was calculated and categorized into four PDE4D7 score classes as described in Alves de Inda M. et al., “Validation of Cyclic Adenosine Monophosphate Phosphodiesterase-4D7 for its Independent Contribution to Risk Stratification in a Prostate Cancer Patient Cohort with Longitudinal Biological Outcomes”, Eur Urol Focus, Vol. 4, No. 3, pages 376-384, 2018. PDE4D7 score class 1 represents patient samples with lowest expression levels of PDE4D7, whereas PDE4D7 score class 4 represents patient samples with highest levels of PDE4D7 expression. RNASeq expression data (TPM—Transcripts Per Million) of the 538 prostate cancer subjects was then investigated for differential gene expression between the PDE4D7 score classes 1 and 4. In particular, it was determined for around 20,000 protein coding transcripts whether the mean expression level of the PDE4D7 score class 1 patients was more than twice as high as the mean expression level of the PDE4D7 score class 4 patients. This analysis resulted in 637 genes with a ratio PDE4D7 score class 1/PDE4D7 score class 4 of >2 with a minimum mean expression of 1 TPM in each of the four PDE4D7 score classes. These 637 genes were then further subjected to molecular pathway analysis (www.david.ncifcrf.gov), which resulted in a range of enriched annotation clusters. The annotation cluster #6 demonstrated enrichment (enrichment score: 5.9) in 17 genes with a function in primary immune deficiency and activation of T-Cell receptor signaling. A further heat map analysis confirmed that these T-Cell receptor signaling genes were generally higher expressed in samples from patients in PDE4D7 score class 1 than from patients in PDE4D7 score class 4.

### PDE4D7 Correlated Genes

The identified PDE4D7 correlated genes ABCC5, CUX2, KIAA1549, PDE4D, RAP1GAP2, SLC39A11, TDRD1, and VWA2 were identified as follows: We have identified in RNAseq data generated on 571 prostate cancer patients on close to 60,000 transcripts a range of genes that are correlated to the expression of the known biomarker PDE4D7 in this data. The correlation between the expression of any of these genes and PDE4D7 across the 571 samples was done by Pearson correlation and is expressed as a value between 0 to 1 in case of positive correlation or a value between −1 to 0 in case of negative correlation.

The equation for the correlation coefficient is:

$$Corr ( X , Y ) = \frac{\sum ( x - \overline{x} ) ( y - \overline{y} )}{\sqrt{\sum {( x - \overline{x} )}^{2} \sum {( y - \overline{y} )}^{2}}} \qquad ( 1 )$$

where x̅ and y̅ are the sample means AVERAGE(PDE4D7) and AVERAGE(gene) across all samples, respectively. As input data for the calculation of the correlation coefficient we used the PDE4D7 score (see Alves de Inda M. et al., 2018, ibid) and the RNAseq determined TPM gene expression value per gene of interest (see below).

The maximum negative correlation coefficient identified between the expression of any of the approximately 60,000 transcripts and the expression of PDE4D7 was −0.38 while the maximum positive correlation coefficient identified between the expression of any of the approximately 60,000 transcripts and the expression of PDE4D7 was +0.56. We selected genes in the range of correlation −0.31 to −0.38 as well as +0.41 to +0.56. We identified in total 77 transcripts matching these characteristics. From those 77 transcripts we selected the eight PDE4D7 correlated genes ABCC5, CUX2, KIAA1549, PDE4D, RAP1GAP2, SLC39A11, TDRD1, and VWA2 by testing Cox regression combination models iteratively in a sub-cohort of 186 patients who were undergoing salvage radiation treatment (SRT) due to post-surgical biochemical relapse. The clinical endpoint tested was prostate cancer specific death after start of SRT. The boundary condition for the selection of the eight genes was given by the restriction that the p-values in the multivariate Cox-regression were <0.1 for all genes retained in the model.

The term “ABCC5” refers to the human ATP binding cassette subfamily C member 5 gene (Ensembl: ENSG00000114770), for example, to the sequence as defined in NCBI Reference Sequence NM_001023587.2 or in NCBI Reference Sequence NM_005688.3, specifically, to the nucleotide sequence as set forth in SEQ ID NO:1 or in SEQ ID NO:2, which correspond to the sequences of the above indicated NCBI Reference Sequences of the ABCC5 transcript, and also relates to the corresponding amino acid sequence for example as set forth in SEQ ID NO:3 or in SEQ ID NO:4, which correspond to the protein sequences defined in NCBI Protein Accession Reference Sequence NP_001018881.1 and in NCBI Protein Accession Reference Sequence NP_005679 encoding the ABCC5 polypeptide.

The term “ABCC5” also comprises nucleotide sequences showing a high degree of homology to ABCC5, e.g., nucleic acid sequences being at least 75%, 80%, 85%, 90%, 91%, 92%, 93%, 94%, 95%, 96%, 97%, 98% or 99% identical to the sequence as set forth in SEQ ID NO:1 or in SEQ ID NO:2 or amino acid sequences being at least 75%, 80%, 85%, 90%, 91%, 92%, 93%, 94%, 95%, 96%, 97%, 98% or 99% identical to the sequence as set forth in SEQ ID NO:3 or in SEQ ID NO:4 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:3 or in SEQ ID NO:4 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:1 or in SEQ ID NO:2.

The term “AIM2” refers to the Absent in Melanoma 2 gene (Ensembl: ENSG00000163568), for example, to the sequence as defined in NCBI Reference Sequence NM_004833, specifically, to the nucleotide sequence as set forth in SEQ ID NO:5, which corresponds to the sequence of the above indicated NCBI Reference Sequence of the AIM2 transcript, and also relates to the corresponding amino acid sequence for example as set forth in SEQ ID NO:6, which corresponds to the protein sequence defined in NCBI Protein Accession Reference Sequence NP_004824 encoding the AIM2 polypeptide.

The term “AIM2” also comprises nucleotide sequences showing a high degree of homology to AIM2, e.g., nucleic acid sequences being at least 75%, 80%, 85%, 90%, 91%, 92%, 93%, 94%, 95%, 96%, 97%, 98% or 99% identical to the sequence as set forth in SEQ ID NO:5 or amino acid sequences being at least 75%, 80%, 85%, 90%, 91%, 92%, 93%, 94%, 95%, 96%, 97%, 98% or 99% identical to the sequence as set forth in SEQ ID NO:6 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:6 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:5.

The term “APOBEC3A” refers to the Apolipoprotein B mRNA Editing Enzyme Catalytic Subunit 3A gene (Ensembl: ENSG00000128383), for example, to the sequence as defined in NCBI Reference Sequence NM_145699, specifically, to the nucleotide sequence as set forth in SEQ ID NO:7, which corresponds to the sequence of the above indicated NCBI Reference Sequence of the APOBEC3A transcript, and also relates to the corresponding amino acid sequence for example as set forth in SEQ ID NO:8, which corresponds to the protein sequence defined in NCBI Protein Accession Reference Sequence NP_663745 encoding the APOBEC3A polypeptide.

The term “APOBEC3A” also comprises nucleotide sequences showing a high degree of homology to APOBEC3A, e.g., nucleic acid sequences being at least 75%, 80%, 85%, 90%, 91%, 92%, 93%, 94%, 95%, 96%, 97%, 98% or 99% identical to the sequence as set forth in SEQ ID NO:7 or amino acid sequences being at least 75%, 80%, 85%, 90%, 91%, 92%, 93%, 94%, 95%, 96%, 97%, 98% or 99% identical to the sequence as set forth in SEQ ID NO: 8 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:8 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:7.

The term “CD2” refers to the Cluster Of Differentiation 2 gene (Ensembl: ENSG00000116824), for example, to the sequence as defined in NCBI Reference Sequence NM_001767, specifically, to the nucleotide sequence as set forth in SEQ ID NO:9, which corresponds to the sequence of the above indicated NCBI Reference Sequence of the CD2 transcript, and also relates to the corresponding amino acid sequence for example as set forth in SEQ ID NO:10, which corresponds to the protein sequence defined in NCBI Protein Accession Reference Sequence NP_001758 encoding the CD2 polypeptide.

The term “CD2” also comprises nucleotide sequences showing a high degree of homology to CD2, e.g., nucleic acid sequences being at least 75%, 80%, 85%, 90%, 91%, 92%, 93%, 94%, 95%, 96%, 97%, 98% or 99% identical to the sequence as set forth in SEQ ID NO:9 or amino acid sequences being at least 75%, 80%, 85%, 90%, 91%, 92%, 93%, 94%, 95%, 96%, 97%, 98% or 99% identical to the sequence as set forth in SEQ ID NO:10 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:10 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:9.

The term “CD247” refers to the Cluster Of Differentiation 247 gene (Ensembl: ENSG00000198821), for example, to the sequence as defined in NCBI Reference Sequence NM_000734 or in NCBI Reference Sequence NM_198053, specifically, to the nucleotide sequence as set forth in SEQ ID NO:11 or in SEQ ID NO:12, which correspond to the sequences of the above indicated NCBI Reference Sequences of the CD247 transcript, and also relates to the corresponding amino acid sequence for example as set forth in SEQ ID NO:13 or in SEQ ID NO:14, which correspond to the protein sequences defined in NCBI Protein Accession Reference Sequence NP_000725 and in NCBI Protein Accession Reference Sequence NP_932170 encoding the CD247 polypeptide.

The term “CD247” also comprises nucleotide sequences showing a high degree of homology to CD247, e.g., nucleic acid sequences being at least 75%, 80%, 85%, 90%, 91%, 92%, 93%, 94%, 95%, 96%, 97%, 98% or 99% identical to the sequence as set forth in SEQ ID NO:11 or in SEQ ID NO:12 or amino acid sequences being at least 75%, 80%, 85%, 90%, 91%, 92%, 93%, 94%, 95%, 96%, 97%, 98% or 99% identical to the sequence as set forth in SEQ ID NO:13 or in SEQ ID NO:14 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:13 or in SEQ ID NO:14 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:11 or in SEQ ID NO:12.

The term “CD28” refers to the Cluster Of Differentiation 28 gene (Ensembl: ENSG00000178562), for example, to the sequence as defined in NCBI Reference Sequence NM_006139 or in NCBI Reference Sequence NM_001243078, specifically, to the nucleotide sequence as set forth in SEQ ID NO:15 or in SEQ ID NO:16, which correspond to the sequences of the above indicated NCBI Reference Sequences of the CD28 transcript, and also relates to the corresponding amino acid sequence for example as set forth in SEQ ID NO:17 or in SEQ ID NO:18, which correspond to the protein sequences defined in NCBI Protein Accession Reference Sequence NP_006130 and in NCBI Protein Accession Reference Sequence NP_001230007 encoding the CD28 polypeptide.

The term “CD28” also comprises nucleotide sequences showing a high degree of homology to CD28, e.g., nucleic acid sequences being at least 75%, 80%, 85%, 90%, 91%, 92%, 93%, 94%, 95%, 96%, 97%, 98% or 99% identical to the sequence as set forth in SEQ ID NO:15 or in SEQ ID NO:16 or amino acid sequences being at least 75%, 80%, 85%, 90%, 91%, 92%, 93%, 94%, 95%, 96%, 97%, 98% or 99% identical to the sequence as set forth in SEQ ID NO:17 or in SEQ ID NO:18 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:17 or in SEQ ID NO:18 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:15 or in SEQ ID NO:16.

The term “CD3E” refers to the Cluster Of Differentiation 3E gene (Ensembl: ENSG00000198851), for example, to the sequence as defined in NCBI Reference Sequence NM_000733, specifically, to the nucleotide sequence as set forth in SEQ ID NO:19, which corresponds to the sequence of the above indicated NCBI Reference Sequence of the CD3E transcript, and also relates to the corresponding amino acid sequence for example as set forth in SEQ ID NO:20, which corresponds to the protein sequence defined in NCBI Protein Accession Reference Sequence NP_000724 encoding the CD3E polypeptide.

The term “CD3E” also comprises nucleotide sequences showing a high degree of homology to CD3E, e.g., nucleic acid sequences being at least 75%, 80%, 85%, 90%, 91%, 92%, 93%, 94%, 95%, 96%, 97%, 98% or 99% identical to the sequence as set forth in SEQ ID NO:19 or amino acid sequences being at least 75%, 80%, 85%, 90%, 91%, 92%, 93%, 94%, 95%, 96%, 97%, 98% or 99% identical to the sequence as set forth in SEQ ID NO:20 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:20 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:19.

The term “CD3G” refers to the Cluster Of Differentiation 3G gene (Ensembl: ENSG00000160654), for example, to the sequence as defined in NCBI Reference Sequence NM_000073, specifically, to the nucleotide sequence as set forth in SEQ ID NO:21, which corresponds to the sequence of the above indicated NCBI Reference Sequence of the CD3G transcript, and also relates to the corresponding amino acid sequence for example as set forth in SEQ ID NO:22, which corresponds to the protein sequence defined in NCBI Protein Accession Reference Sequence NP_000064 encoding the CD3G polypeptide.

The term “CD3G” also comprises nucleotide sequences showing a high degree of homology to CD3G, e.g., nucleic acid sequences being at least 75%, 80%, 85%, 90%, 91%, 92%, 93%, 94%, 95%, 96%, 97%, 98% or 99% identical to the sequence as set forth in SEQ ID NO:21 or amino acid sequences being at least 75%, 80%, 85%, 90%, 91%, 92%, 93%, 94%, 95%, 96%, 97%, 98% or 99% identical to the sequence as set forth in SEQ ID NO:22 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:22 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:21.

The term “CD4” refers to the Cluster Of Differentiation 4 gene (Ensembl: ENSG00000010610), for example, to the sequence as defined in NCBI Reference Sequence NM_000616, specifically, to the nucleotide sequence as set forth in SEQ ID NO:23, which corresponds to the sequence of the above indicated NCBI Reference Sequence of the CD4 transcript, and also relates to the corresponding amino acid sequence for example as set forth in SEQ ID NO:24, which corresponds to the protein sequence defined in NCBI Protein Accession Reference Sequence NP_000607 encoding the CD4 polypeptide.

The term “CD4” also comprises nucleotide sequences showing a high degree of homology to CD4, e.g., nucleic acid sequences being at least 75%, 80%, 85%, 90%, 91%, 92%, 93%, 94%, 95%, 96%, 97%, 98% or 99% identical to the sequence as set forth in SEQ ID NO:23 or amino acid sequences being at least 75%, 80%, 85%, 90%, 91%, 92%, 93%, 94%, 95%, 96%, 97%, 98% or 99% identical to the sequence as set forth in SEQ ID NO:24 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:24 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:23.

The term “CIAO1” refers to the Cytosolic Iron-Sulfur Assembly Component 1 gene (Ensembl: ENSG00000144021), for example, to the sequence as defined in NCBI Reference Sequence NM_004804, specifically, to the nucleotide sequence as set forth in SEQ ID NO:25, which corresponds to the sequence of the above indicated NCBI Reference Sequence of the CIAO1 transcript, and also relates to the corresponding amino acid sequence for example as set forth in SEQ ID NO:26, which corresponds to the protein sequence defined in NCBI Protein Accession Reference Sequence NP_663745 encoding the CIAO1 polypeptide.

The term “CIAO1” also comprises nucleotide sequences showing a high degree of homology to CIAO1, e.g., nucleic acid sequences being at least 75%, 80%, 85%, 90%, 91%, 92%, 93%, 94%, 95%, 96%, 97%, 98% or 99% identical to the sequence as set forth in SEQ ID NO:25 or amino acid sequences being at least 75%, 80%, 85%, 90%, 91%, 92%, 93%, 94%, 95%, 96%, 97%, 98% or 99% identical to the sequence as set forth in SEQ ID NO:26 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:26 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:25.

The term “CSK” refers to the C-Terminal Src Kinase gene (Ensembl: ENSG00000103653), for example, to the sequence as defined in NCBI Reference Sequence NM_004383, specifically, to the nucleotide sequence as set forth in SEQ ID NO:27, which corresponds to the sequence of the above indicated NCBI Reference Sequence of the CSK transcript, and also relates to the corresponding amino acid sequence for example as set forth in SEQ ID NO:28, which corresponds to the protein sequence defined in NCBI Protein Accession Reference Sequence NP_004374 encoding the CSK polypeptide.

The term “CSK” also comprises nucleotide sequences showing a high degree of homology to CSK, e.g., nucleic acid sequences being at least 75%, 80%, 85%, 90%, 91%, 92%, 93%, 94%, 95%, 96%, 97%, 98% or 99% identical to the sequence as set forth in SEQ ID NO:27 or amino acid sequences being at least 75%, 80%, 85%, 90%, 91%, 92%, 93%, 94%, 95%, 96%, 97%, 98% or 99% identical to the sequence as set forth in SEQ ID NO:28 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:28 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:27.

The term “CUX2” refers to the human Cut Like Homeobox 2 gene (Ensembl: ENSG00000111249), for example, to the sequence as defined in NCBI Reference Sequence NM_015267.3, specifically, to the nucleotide sequence as set forth in SEQ ID NO:29, which corresponds to the sequence of the above indicated NCBI Reference Sequence of the CUX2 transcript, and also relates to the corresponding amino acid sequence for example as set forth in SEQ ID NO:30, which corresponds to the protein sequence defined in NCBI Protein Accession Reference Sequence NP_056082.2 encoding the CUX2 polypeptide.

The term “CUX2” also comprises nucleotide sequences showing a high degree of homology to CUX2, e.g., nucleic acid sequences being at least 75%, 80%, 85%, 90%, 91%, 92%, 93%, 94%, 95%, 96%, 97%, 98% or 99% identical to the sequence as set forth in SEQ ID NO:29 or amino acid sequences being at least 75%, 80%, 85%, 90%, 91%, 92%, 93%, 94%, 95%, 96%, 97%, 98% or 99% identical to the sequence as set forth in SEQ ID NO:30 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:30 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:29.

The term “DDX58” refers to the DExD/H-box Helicase 58 gene (Ensembl: ENSG00000107201), for example, to the sequence as defined in NCBI Reference Sequence NM_014314, specifically, to the nucleotide sequence as set forth in SEQ ID NO:31, which corresponds to the sequence of the above indicated NCBI Reference Sequence of the DDX58 transcript, and also relates to the corresponding amino acid sequence for example as set forth in SEQ ID NO:32, which corresponds to the protein sequence defined in NCBI Protein Accession Reference Sequence NP_055129 encoding the DDX58 polypeptide.

The term “DDX58” also comprises nucleotide sequences showing a high degree of homology to DDX58, e.g., nucleic acid sequences being at least 75%, 80%, 85%, 90%, 91%, 92%, 93%, 94%, 95%, 96%, 97%, 98% or 99% identical to the sequence as set forth in SEQ ID NO:31 or amino acid sequences being at least 75%, 80%, 85%, 90%, 91%, 92%, 93%, 94%, 95%, 96%, 97%, 98% or 99% identical to the sequence as set forth in SEQ ID NO:32 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:32 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:31.

The term “DHX9” refers to the DExD/H-box Helicase 9 gene (Ensembl: ENSG00000135829), for example, to the sequence as defined in NCBI Reference Sequence NM_001357, specifically, to the nucleotide sequence as set forth in SEQ ID NO:33, which corresponds to the sequence of the above indicated NCBI Reference Sequence of the DHX9 transcript, and also relates to the corresponding amino acid sequence for example as set forth in SEQ ID NO:34, which corresponds to the protein sequence defined in NCBI Protein Accession Reference Sequence NP_001348 encoding the DHX9 polypeptide.

The term “DHX9” also comprises nucleotide sequences showing a high degree of homology to DHX9, e.g., nucleic acid sequences being at least 75%, 80%, 85%, 90%, 91%, 92%, 93%, 94%, 95%, 96%, 97%, 98% or 99% identical to the sequence as set forth in SEQ ID NO:33 or amino acid sequences being at least 75%, 80%, 85%, 90%, 91%, 92%, 93%, 94%, 95%, 96%, 97%, 98% or 99% identical to the sequence as set forth in SEQ ID NO:34 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:34 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:33.

The term “EZR” refers to the Ezrin gene (Ensembl: ENSG00000092820), for example, to the sequence as defined in NCBI Reference Sequence NM_003379, specifically, to the nucleotide sequence as set forth in SEQ ID NO:35, which corresponds to the sequence of the above indicated NCBI Reference Sequence of the EZR transcript, and also relates to the corresponding amino acid sequence for example as set forth in SEQ ID NO:36, which corresponds to the protein sequence defined in NCBI Protein Accession Reference Sequence NP_003370 encoding the EZR polypeptide.

The term “EZR” also comprises nucleotide sequences showing a high degree of homology to EZR, e.g., nucleic acid sequences being at least 75%, 80%, 85%, 90%, 91%, 92%, 93%, 94%, 95%, 96%, 97%, 98% or 99% identical to the sequence as set forth in SEQ ID NO:35 or amino acid sequences being at least 75%, 80%, 85%, 90%, 91%, 92%, 93%, 94%, 95%, 96%, 97%, 98% or 99% identical to the sequence as set forth in SEQ ID NO:36 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:36 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:35.

The term “FYN” refers to the FYN Proto-Oncogene gene (Ensembl: ENSG00000010810), for example, to the sequence as defined in NCBI Reference Sequence NM_002037 or in NCBI Reference Sequence NM_153047 or in NCBI Reference Sequence NM_153048, specifically, to the nucleotide sequence as set forth in SEQ ID NO:37 or in SEQ ID NO:38 or in SEQ ID NO:39, which correspond to the sequences of the above indicated NCBI Reference Sequences of the FYN transcript, and also relates to the corresponding amino acid sequence for example as set forth in SEQ ID NO:40 or in SEQ ID NO:41 or in SEQ ID NO:42, which correspond to the protein sequences defined in NCBI Protein Accession Reference Sequence NP_002028 and in NCBI Protein Accession Reference Sequence NP_694592 and in NCBI Protein Accession Reference Sequence XP_005266949 encoding the FYN polypeptide.

The term “FYN” also comprises nucleotide sequences showing a high degree of homology to FYN, e.g., nucleic acid sequences being at least 75%, 80%, 85%, 90%, 91%, 92%, 93%, 94%, 95%, 96%, 97%, 98% or 99% identical to the sequence as set forth in SEQ ID NO:37 or in SEQ ID NO:38 or in SEQ ID NO:39 or amino acid sequences being at least 75%, 80%, 85%, 90%, 91%, 92%, 93%, 94%, 95%, 96%, 97%, 98% or 99% identical to the sequence as set forth in SEQ ID NO:40 or in SEQ ID NO:41 or in SEQ ID NO:42 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:40 or in SEQ ID NO:41 or in SEQ ID NO:42 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:37 or in SEQ ID NO:38 or in SEQ ID NO:39.

The term “IFI16” refers to the Interferon Gamma Inducible Protein 16 gene (Ensembl: ENSG00000163565), for example, to the sequence as defined in NCBI Reference Sequence NM_005531, specifically, to the nucleotide sequence as set forth in SEQ ID NO:43, which corresponds to the sequence of the above indicated NCBI Reference Sequence of the IFI16 transcript, and also relates to the corresponding amino acid sequence for example as set forth in SEQ ID NO:44, which corresponds to the protein sequence defined in NCBI Protein Accession Reference Sequence NP_005522 encoding the IFI16 polypeptide.

The term “IFI16” also comprises nucleotide sequences showing a high degree of homology to IFI16, e.g., nucleic acid sequences being at least 75%, 80%, 85%, 90%, 91%, 92%, 93%, 94%, 95%, 96%, 97%, 98% or 99% identical to the sequence as set forth in SEQ ID NO:43 or amino acid sequences being at least 75%, 80%, 85%, 90%, 91%, 92%, 93%, 94%, 95%, 96%, 97%, 98% or 99% identical to the sequence as set forth in SEQ ID NO:44 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:44 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:43.

The term “IFIH1” refers to the Interferon Induced With Helicase C Domain 1 gene (Ensembl: ENSG00000115267), for example, to the sequence as defined in NCBI Reference Sequence NM_022168, specifically, to the nucleotide sequence as set forth in SEQ ID NO:45, which corresponds to the sequence of the above indicated NCBI Reference Sequence of the IFIH1 transcript, and also relates to the corresponding amino acid sequence for example as set forth in SEQ ID NO:46, which corresponds to the protein sequence defined in NCBI Protein Accession Reference Sequence NP_071451 encoding the IFIH1 polypeptide.

The term “IFIH1” also comprises nucleotide sequences showing a high degree of homology to IFIH1, e.g., nucleic acid sequences being at least 75%, 80%, 85%, 90%, 91%, 92%, 93%, 94%, 95%, 96%, 97%, 98% or 99% identical to the sequence as set forth in SEQ ID NO:45 or amino acid sequences being at least 75%, 80%, 85%, 90%, 91%, 92%, 93%, 94%, 95%, 96%, 97%, 98% or 99% identical to the sequence as set forth in SEQ ID NO:46 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:46 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:45.

The term “IFIT1” refers to the Interferon Induced Protein With Tetratricopeptide Repeats 1 gene (Ensembl: ENSG00000185745), for example, to the sequence as defined in NCBI Reference Sequence NM_001270929 or in NCBI Reference Sequence NM_001548.5, specifically, to the nucleotide sequence as set forth in SEQ ID NO:47 or in SEQ ID NO:48, which correspond to the sequences of the above indicated NCBI Reference Sequences of the IFIT1 transcript, and also relates to the corresponding amino acid sequence for example as set forth in SEQ ID NO:49 or in SEQ ID NO:50, which correspond to the protein sequences defined in NCBI Protein Accession Reference Sequence NP_001257858 and in NCBI Protein Accession Reference Sequence NP_001539 encoding the IFIT1 polypeptide.

The term “IFIT1” also comprises nucleotide sequences showing a high degree of homology to IFIT1, e.g., nucleic acid sequences being at least 75%, 80%, 85%, 90%, 91%, 92%, 93%, 94%, 95%, 96%, 97%, 98% or 99% identical to the sequence as set forth in SEQ ID NO:47 or in SEQ ID NO:48 or amino acid sequences being at least 75%, 80%, 85%, 90%, 91%, 92%, 93%, 94%, 95%, 96%, 97%, 98% or 99% identical to the sequence as set forth in SEQ ID NO:49 or in SEQ ID NO:50 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:49 or SEQ ID NO:50 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:47 or in SEQ ID NO:48.

The term “IFIT3” refers to the Interferon Induced Protein With Tetratricopeptide Repeats 3 gene (Ensembl: ENSG00000119917), for example, to the sequence as defined in NCBI Reference Sequence NM_001031683, specifically, to the nucleotide sequence as set forth in SEQ ID NO:51, which corresponds to the sequence of the above indicated NCBI Reference Sequence of the IFIT3 transcript, and also relates to the corresponding amino acid sequence for example as set forth in SEQ ID NO:52, which corresponds to the protein sequence defined in NCBI Protein Accession Reference Sequence NP_001026853 encoding the IFIT3 polypeptide.

The term “IFIT3” also comprises nucleotide sequences showing a high degree of homology to IFIT3, e.g., nucleic acid sequences being at least 75%, 80%, 85%, 90%, 91%, 92%, 93%, 94%, 95%, 96%, 97%, 98% or 99% identical to the sequence as set forth in SEQ ID NO:51 or amino acid sequences being at least 75%, 80%, 85%, 90%, 91%, 92%, 93%, 94%, 95%, 96%, 97%, 98% or 99% identical to the sequence as set forth in SEQ ID NO:52 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:52 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:51.

The term “KIAA1549” refers to the human KIAA1549 gene (Ensembl: ENSG00000122778), for example, to the sequence as defined in NCBI Reference Sequence NM_020910 or in NCBI Reference Sequence NM_001164665, specifically, to the nucleotide sequence as set forth in SEQ ID NO:53 or in SEQ ID NO:54, which correspond to the sequences of the above indicated NCBI Reference Sequence of the KIAA1549 transcript, and also relates to the corresponding amino acid sequence for example as set forth in SEQ ID NO:55 or in SEQ ID NO:56, which correspond to the protein sequences defined in NCBI Protein Accession Reference Sequence NP_065961 and in NCBI Protein Accession Reference Sequence NP_001158137 encoding the KIAA1549 polypeptide.

The term “KIAA1549” also comprises nucleotide sequences showing a high degree of homology to KIAA1549, e.g., nucleic acid sequences being at least 75%, 80%, 85%, 90%, 91%, 92%, 93%, 94%, 95%, 96%, 97%, 98% or 99% identical to the sequence as set forth in SEQ ID NO:53 or in SEQ ID NO:54 or amino acid sequences being at least 75%, 80%, 85%, 90%, 91%, 92%, 93%, 94%, 95%, 96%, 97%, 98% or 99% identical to the sequence as set forth in SEQ ID NO:55 or in SEQ ID NO:56 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:55 or in SEQ ID NO:56 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:53 or in SEQ ID NO:54.

The term “LAT” refers to the Linker For Activation Of T-Cells gene (Ensembl: ENSG00000213658), for example, to the sequence as defined in NCBI Reference Sequence NM_ 001014987 or in NCBI Reference Sequence NM_014387, specifically, to the nucleotide sequence as set forth in SEQ ID NO:57 or in SEQ ID NO:58, which correspond to the sequences of the above indicated NCBI Reference Sequences of the LAT transcript, and also relates to the corresponding amino acid sequence for example as set forth in SEQ ID NO:59 or in SEQ ID NO:60, which corresponds to the protein sequences defined in NCBI Protein Accession Reference Sequence NP_001014987 and in NCBI Protein Accession Reference Sequence NP_055202 encoding the LAT polypeptide.

The term “LAT” also comprises nucleotide sequences showing a high degree of homology to LAT, e.g., nucleic acid sequences being at least 75%, 80%, 85%, 90%, 91%, 92%, 93%, 94%, 95%, 96%, 97%, 98% or 99% identical to the sequence as set forth in SEQ ID NO:57 or in SEQ ID NO:58 or amino acid sequences being at least 75%, 80%, 85%, 90%, 91%, 92%, 93%, 94%, 95%, 96%, 97%, 98% or 99% identical to the sequence as set forth in SEQ ID NO:59 or in SEQ ID NO:60 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:59 or in SEQ ID NO:60 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:57 or in SEQ ID NO:58.

The term “LCK” refers to the LCK Proto-Oncogene gene (Ensembl: ENSG00000182866), for example, to the sequence as defined in NCBI Reference Sequence NM_005356, specifically, to the nucleotide sequence as set forth in SEQ ID NO:61, which corresponds to the sequence of the above indicated NCBI Reference Sequence of the LCK transcript, and also relates to the corresponding amino acid sequence for example as set forth in SEQ ID NO:62, which corresponds to the protein sequence defined in NCBI Protein Accession Reference Sequence NP_005347 encoding the LCK polypeptide.

The term “LCK” also comprises nucleotide sequences showing a high degree of homology to LCK, e.g., nucleic acid sequences being at least 75%, 80%, 85%, 90%, 91%, 92%, 93%, 94%, 95%, 96%, 97%, 98% or 99% identical to the sequence as set forth in SEQ ID NO:61 or amino acid sequences being at least 75%, 80%, 85%, 90%, 91%, 92%, 93%, 94%, 95%, 96%, 97%, 98% or 99% identical to the sequence as set forth in SEQ ID NO:62 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:62 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:61.

The term “LRRFIP1” refers to the LRR Binding FLII Interacting Protein 1 gene (Ensembl: ENSG00000124831), for example, to the sequence as defined in NCBI Reference Sequence NM_004735 or in NCBI Reference Sequence NM_001137550 or in NCBI Reference Sequence NM_001137553 or in NCBI Reference Sequence NM_001137552, specifically, to the nucleotide sequence as set forth in SEQ ID NO:63 or in SEQ ID NO:64 or in SEQ ID NO:65 or in SEQ ID NO:66, which correspond to the sequences of the above indicated NCBI Reference Sequences of the LRRFIP1 transcript, and also relates to the corresponding amino acid sequence for example as set forth in SEQ ID NO:67 or in SEQ ID NO:68 or in SEQ ID NO:69 or in SEQ ID NO:70, which correspond to the protein sequences defined in NCBI Protein Accession Reference Sequence NP_004726 and in NCBI Protein Accession Reference Sequence NP_001131022 and in NCBI Protein Accession Reference Sequence NP_001131025 and in NCBI Protein Accession Reference Sequence NP_001131024 encoding the LRRFIP1 polypeptide.

The term “LRRFIP1” also comprises nucleotide sequences showing a high degree of homology to LRRFIP1, e.g., nucleic acid sequences being at least 75%, 80%, 85%, 90%, 91%, 92%, 93%, 94%, 95%, 96%, 97%, 98% or 99% identical to the sequence as set forth in SEQ ID NO:63 or in SEQ ID NO:64 or in SEQ ID NO:65 or in SEQ ID NO:66 or amino acid sequences being at least 75%, 80%, 85%, 90%, 91%, 92%, 93%, 94%, 95%, 96%, 97%, 98% or 99% identical to the sequence as set forth in SEQ ID NO:67 or in SEQ ID NO:68 or in SEQ ID NO:69 or in SEQ ID NO:70 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:67 or in SEQ ID NO:68 or in SEQ ID NO:69 or in SEQ ID NO:70 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:63 or in SEQ ID NO:64 or in SEQ ID NO:65 or in SEQ ID NO:66.

The term “MYD88” refers to the MYD88 Innate Immune Signal Transduction Adaptor gene (Ensembl: ENSG00000172936), for example, to the sequence as defined in NCBI Reference Sequence NM_001172567 or in NCBI Reference Sequence NM_001172568 or in NCBI Reference Sequence NM_001172569 or in NCBI Reference Sequence NM_001172566 or in NCBI Reference Sequence NM_002468, specifically, to the nucleotide sequences as set forth in SEQ ID NO:71 or in SEQ ID NO:72 or in SEQ ID NO:73 or in SEQ ID NO:74 or in SEQ ID NO:75, which correspond to the sequences of the above indicated NCBI Reference Sequences of the MYD88 transcript, and also relates to the corresponding amino acid sequence for example as set forth in SEQ ID NO:76 or in SEQ ID NO:77 or in SEQ ID NO:78 or in SEQ ID NO:79 or in SEQ ID NO:80, which correspond to the protein sequences defined in NCBI Protein Accession Reference Sequence NP_001166038 and in NCBI Protein Accession Reference Sequence NP_001166039 and in NCBI Protein Accession Reference Sequence NP_001166040 and in NCBI Protein Accession Reference Sequence NP_001166037 and in NCBI Protein Accession Reference Sequence NP_002459 encoding the MYD88 polypeptide.

The term “MYD88” also comprises nucleotide sequences showing a high degree of homology to MYD88, e.g., nucleic acid sequences being at least 75%, 80%, 85%, 90%, 91%, 92%, 93%, 94%, 95%, 96%, 97%, 98% or 99% identical to the sequence as set forth in SEQ ID NO:71 or in SEQ ID NO:72 or in SEQ ID NO:73 or in SEQ ID NO:74 or in SEQ ID NO:75 or amino acid sequences being at least 75%, 80%, 85%, 90%, 91%, 92%, 93%, 94%, 95%, 96%, 97%, 98% or 99% identical to the sequence as set forth in SEQ ID NO:76 or in SEQ ID NO:77 or in SEQ ID NO:78 or in SEQ ID NO:79 or in SEQ ID NO:80 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:76 or in SEQ ID NO:77 or in SEQ ID NO:78 or in SEQ ID NO:79 or in SEQ ID NO:80 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:71 or in SEQ ID NO:72 or in SEQ ID NO:73 or in SEQ ID NO:74 or in SEQ ID NO:75.

The term “OAS1” refers to the 2′-5′-Oligoadenylate Synthetase 1 gene (Ensembl: ENSG00000089127), for example, to the sequence as defined in NCBI Reference Sequence NM_001320151 or in NCBI Reference Sequence NM_002534 or in NCBI Reference Sequence NM_001032409 or in NCBI Reference Sequence NM_016816, specifically, to the nucleotide sequences as set forth in SEQ ID NO:81 or in SEQ ID NO:82 or in SEQ ID NO:83 or in SEQ ID NO:84, which correspond to the sequences of the above indicated NCBI Reference Sequences of the OAS1 transcript, and also relates to the corresponding amino acid sequences for example as set forth in SEQ ID NO:85 or in SEQ ID NO:86 or in SEQ ID NO:87 or in SEQ ID NO:88, which correspond to the protein sequences defined in NCBI Protein Accession Reference Sequence NP_001307080 and in NCBI Protein Accession Reference Sequence NP_002525 and in NCBI Protein Accession Reference Sequence NP_001027581 and in NCBI Protein Accession Reference Sequence NP_058132 encoding the OAS1 polypeptide.

The term “OAS1” also comprises nucleotide sequences showing a high degree of homology to OAS1, e.g., nucleic acid sequences being at least 75%, 80%, 85%, 90%, 91%, 92%, 93%, 94%, 95%, 96%, 97%, 98% or 99% identical to the sequence as set forth in SEQ ID NO:81 or in SEQ ID NO:82 or in SEQ ID NO:83 or in SEQ ID NO:84 or amino acid sequences being at least 75%, 80%, 85%, 90%, 91%, 92%, 93%, 94%, 95%, 96%, 97%, 98% or 99% identical to the sequence as set forth in SEQ ID NO:85 or in SEQ ID NO:86 or in SEQ ID NO:87 or in SEQ ID NO:88 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:85 or in SEQ ID NO:86 or in SEQ ID NO:87 or in SEQ ID NO:88 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:81 or in SEQ ID NO:82 or in SEQ ID NO:83 or in SEQ ID NO:84.

The term “PAG1” refers to the Phosphoprotein Membrane Anchor With Glycosphingolipid Microdomains 1 gene (Ensembl: ENSG00000076641), for example, to the sequence as defined in NCBI Reference Sequence NM_018440, specifically, to the nucleotide sequence as set forth in SEQ ID NO:89, which corresponds to the sequence of the above indicated NCBI Reference Sequence of the PAG1 transcript, and also relates to the corresponding amino acid sequence for example as set forth in SEQ ID NO:90, which corresponds to the protein sequence defined in NCBI Protein Accession Reference Sequence NP_060910 encoding the PAG1 polypeptide.

The term “PAG1” also comprises nucleotide sequences showing a high degree of homology to PAG1, e.g., nucleic acid sequences being at least 75%, 80%, 85%, 90%, 91%, 92%, 93%, 94%, 95%, 96%, 97%, 98% or 99% identical to the sequence as set forth in SEQ ID NO:89 or amino acid sequences being at least 75%, 80%, 85%, 90%, 91%, 92%, 93%, 94%, 95%, 96%, 97%, 98% or 99% identical to the sequence as set forth in SEQ ID NO:90 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:90 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:89.

The term “PDE4D” refers to the human Phosphodiesterase 4D gene (Ensembl: ENSG00000113448), for example, to the sequence as defined in NCBI Reference Sequence NM_001104631 or in NCBI Reference Sequence NM_001349242 or in NCBI Reference Sequence NM_001197218 or in NCBI Reference Sequence NM_006203 or in NCBI Reference Sequence NM_001197221 or in NCBI Reference Sequence NM_001197220 or in NCBI Reference Sequence NM_001197223 or in NCBI Reference Sequence NM_001165899 or in NCBI Reference Sequence NM_001165899, specifically, to the nucleotide sequence as set forth in SEQ ID NO:91 or in SEQ ID NO:92 or in SEQ ID NO:93 or in SEQ ID NO:94 or in SEQ ID NO:95 or in SEQ ID NO:96 or in SEQ ID NO:97 or in SEQ ID NO:98 or in SEQ ID NO:99, which correspond to the sequences of the above indicated NCBI Reference Sequence of the PDE4D transcript, and also relates to the corresponding amino acid sequence for example as set forth in SEQ ID NO:100 or in SEQ ID NO:101 or in SEQ ID NO:102 or in SEQ ID NO:103 or in SEQ ID NO:104 or in SEQ ID NO:105 or in SEQ ID NO:106 or in SEQ ID NO:107 or in SEQ ID NO:108, which correspond to the protein sequences defined in NCBI Protein Accession Reference Sequence NP_001098101 and in NCBI Protein Accession Reference Sequence NP_001336171 and in NCBI Protein Accession Reference Sequence NP_001184147 and in NCBI Protein Accession Reference Sequence NP_006194 and in NCBI Protein Accession Reference Sequence NP_001184150 and in NCBI Protein Accession Reference Sequence NP_001184149 and in NCBI Protein Accession Reference Sequence NP_001184152 and in NCBI Protein Accession Reference Sequence NP_001159371 and in NCBI Protein Accession Reference Sequence NP_001184148 encoding the PDE4D polypeptide.

The term “PDE4D” also comprises nucleotide sequences showing a high degree of homology to PDE4D, e.g., nucleic acid sequences being at least 75%, 80%, 85%, 90%, 91%, 92%, 93%, 94%, 95%, 96%, 97%, 98% or 99% identical to the sequence as set forth in SEQ ID NO:91 or in SEQ ID NO:92 or in SEQ ID NO:93 or in SEQ ID NO:94 or in SEQ ID NO:95 or in SEQ ID NO:96 or in SEQ ID NO:97 or in SEQ ID NO:98 or in SEQ ID NO:99 or amino acid sequences being at least 75%, 80%, 85%, 90%, 91%, 92%, 93%, 94%, 95%, 96%, 97%, 98% or 99% identical to the sequence as set forth in SEQ ID NO:100 or in SEQ ID NO:101 or in SEQ ID NO:102 or in SEQ ID NO:103 or in SEQ ID NO:104 or in SEQ ID NO:105 or in SEQ ID NO:106 or in SEQ ID NO:107 or in SEQ ID NO:108 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:100 or in SEQ ID NO:101 or in SEQ ID NO:102 or in SEQ ID NO:103 or in SEQ ID NO:104 or in SEQ ID NO:105 or in SEQ ID NO:106 or in SEQ ID NO:107 or in SEQ ID NO:108 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:91 or in SEQ ID NO:92 or in SEQ ID NO:93 or in SEQ ID NO:94 or in SEQ ID NO:95 or in SEQ ID NO:96 or in SEQ ID NO:97 or in SEQ ID NO:98 or in SEQ ID NO:99.

The term “PRKACA” refers to the Protein Kinase cAMP-Activated Catalytic Subunit Alpha gene (Ensembl: ENSG00000072062), for example, to the sequence as defined in NCBI Reference Sequence NM_002730 or in NCBI Reference Sequence NM_207518, specifically, to the nucleotide sequences as set forth in SEQ ID NO:109 or in SEQ ID NO:110, which correspond to the sequences of the above indicated NCBI Reference Sequences of the PRKACA transcript, and also relates to the corresponding amino acid sequence for example as set forth in SEQ ID NO:111 or in SEQ ID NO:112, which correspond to the protein sequences defined in NCBI Protein Accession Reference Sequence NP_002721 and in NCBI Protein Accession Reference Sequence NP_997401 encoding the PRKACA polypeptide.

The term “PRKACA” also comprises nucleotide sequences showing a high degree of homology to PRKACA, e.g., nucleic acid sequences being at least 75%, 80%, 85%, 90%, 91%, 92%, 93%, 94%, 95%, 96%, 97%, 98% or 99% identical to the sequence as set forth in SEQ ID NO:109 or in SEQ ID NO:110 or amino acid sequences being at least 75%, 80%, 85%, 90%, 91%, 92%, 93%, 94%, 95%, 96%, 97%, 98% or 99% identical to the sequence as set forth in SEQ ID NO:111 or in SEQ ID NO:112 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:111 or in SEQ ID NO:112 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:109 or in SEQ ID NO:110.

The term “PRKACB” refers to the Protein Kinase cAMP-Activated Catalytic Subunit Beta gene (Ensembl: ENSG00000142875), for example, to the sequence as defined in NCBI Reference Sequence NM_002731 or in NCBI Reference Sequence NM_182948 or in NCBI Reference Sequence NM_001242860 or in NCBI Reference Sequence NM_001242859 or in NCBI Reference Sequence NM_001242858 or in NCBI Reference Sequence NM_001242862 or in NCBI Reference Sequence NM_001242861 or in NCBI Reference Sequence NM_001300915 or in NCBI Reference Sequence NM_207578 or in NCBI Reference Sequence NM_001242857 or in NCBI Reference Sequence NM_001300917, specifically, to the nucleotide sequence as set forth in SEQ ID NO:113 or in SEQ ID NO:114 or in SEQ ID NO:115 or in SEQ ID NO:116 or in SEQ ID NO:117 or in SEQ ID NO:118 or in SEQ ID NO:119 or in SEQ ID NO:120 or in SEQ ID NO:121 or in SEQ ID NO:122 or in SEQ ID NO:123, which correspond to the sequences of the above indicated NCBI Reference Sequences of the PRKACB transcript, and also relates to the corresponding amino acid sequence for example as set forth in SEQ ID NO:124 or in SEQ ID NO:125 or in SEQ ID NO:126 or in SEQ ID NO:127 or in SEQ ID NO:128 or in SEQ ID NO:129 or in SEQ ID NO:130 or in SEQ ID NO:131 or in SEQ ID NO:132 or in SEQ ID NO:133 or in SEQ ID NO:134, which correspond to the protein sequences defined in NCBI Protein Accession Reference Sequence NP_002722 and in NCBI Protein Accession Reference Sequence NP_891993 and in NCBI Protein Accession Reference Sequence NP_001229789 and in NCBI Protein Accession Reference Sequence NP_001229788 and in NCBI Protein Accession Reference Sequence NP_001229787 and in NCBI Protein Accession Reference Sequence NP_001229791 and in NCBI Protein Accession Reference Sequence NP_001229790 and in NCBI Protein Accession Reference Sequence NP_001287844 and in NCBI Protein Accession Reference Sequence NP_997461 and in NCBI Protein Accession Reference Sequence NP_001229786 and in NCBI Protein Accession Reference Sequence NP_001287846 encoding the PRKACB polypeptide.

The term “PRKACB” also comprises nucleotide sequences showing a high degree of homology to PRKACB, e.g., nucleic acid sequences being at least 75%, 80%, 85%, 90%, 91%, 92%, 93%, 94%, 95%, 96%, 97%, 98% or 99% identical to the sequence as set forth in SEQ ID NO:113 or in SEQ ID NO:114 or in SEQ ID NO:115 or in SEQ ID NO:116 or in SEQ ID NO:117 or in SEQ ID NO:118 or in SEQ ID NO:119 or in SEQ ID NO:120 or in SEQ ID NO:121 or in SEQ ID NO:122 or in SEQ ID NO:123 or amino acid sequences being at least 75%, 80%, 85%, 90%, 91%, 92%, 93%, 94%, 95%, 96%, 97%, 98% or 99% identical to the sequence as set forth in SEQ ID NO:124 or in SEQ ID NO:125 or in SEQ ID NO:126 or in SEQ ID NO:127 or in SEQ ID NO:128 or in SEQ ID NO:129 or in SEQ ID NO:130 or in SEQ ID NO:131 or in SEQ ID NO:132 or in SEQ ID NO:133 or in SEQ ID NO:134 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:124 or in SEQ ID NO:125 or in SEQ ID NO:126 or in SEQ ID NO:127 or in SEQ ID NO:128 or in SEQ ID NO:129 or in SEQ ID NO:130 or in SEQ ID NO:131 or in SEQ ID NO:132 or in SEQ ID NO:133 or in SEQ ID NO:134 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:113 or in SEQ ID NO:114 or in SEQ ID NO:115 or in SEQ ID NO:116 or in SEQ ID NO:117 or in SEQ ID NO:118 or in SEQ ID NO:119 or in SEQ ID NO:120 or in SEQ ID NO:121 or in SEQ ID NO:122 or in SEQ ID NO:123.

The term “PTPRC” refers to the Protein Tyrosine Phosphatase Receptor Type C gene (Ensembl: ENSG00000081237), for example, to the sequence as defined in NCBI Reference Sequence NM_002838 or in NCBI Reference Sequence NM_080921, specifically, to the nucleotide sequence as set forth in SEQ ID NO:135 or in SEQ ID NO:136, which correspond to the sequences of the above indicated NCBI Reference Sequences of the PTPRC transcript, and also relates to the corresponding amino acid sequence for example as set forth in SEQ ID NO:137 or in SEQ ID NO:138, which correspond to the protein sequences defined in NCBI Protein Accession Reference Sequence NP_002829 encoding the PTPRC polypeptide and in NCBI Protein Accession Reference Sequence NP_563578.

The term “PTPRC” also comprises nucleotide sequences showing a high degree of homology to PTPRC, e.g., nucleic acid sequences being at least 75%, 80%, 85%, 90%, 91%, 92%, 93%, 94%, 95%, 96%, 97%, 98% or 99% identical to the sequence as set forth in SEQ ID NO:135 or in SEQ ID NO:136 or amino acid sequences being at least 75%, 80%, 85%, 90%, 91%, 92%, 93%, 94%, 95%, 96%, 97%, 98% or 99% identical to the sequence as set forth in SEQ ID NO:137 or in SEQ ID NO:138 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:137 or in SEQ ID NO:138 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:135 or in SEQ ID NO:136.

The term “RAP1GAP2” refers to the human RAP1 GTPase Activating Protein 2 gene (ENSG00000132359), for example, to the sequence as defined in NCBI Reference Sequence NM_015085 or in NCBI Reference Sequence NM_001100398 or in NCBI Reference Sequence NM_001330058, specifically, to the nucleotide sequence as set forth in SEQ ID NO:139 or in SEQ ID NO:140 or in SEQ ID NO:141, which correspond to the sequences of the above indicated NCBI Reference Sequences of the RAP1GAP2 transcript, and also relates to the corresponding amino acid sequence for example as set forth in SEQ ID NO:142 or in SEQ ID NO:143 or in SEQ ID NO:144, which correspond to the protein sequences defined in NCBI Protein Accession Reference Sequence NP_055900 and in NCBI Protein Accession Reference Sequence NP_001093868 and in NCBI Protein Accession Reference Sequence NP_001316987 encoding the RAP1GAP2 polypeptide.

The term “RAP1GAP2” also comprises nucleotide sequences showing a high degree of homology to RAP1GAP2, e.g., nucleic acid sequences being at least 75%, 80%, 85%, 90%, 91%, 92%, 93%, 94%, 95%, 96%, 97%, 98% or 99% identical to the sequence as set forth in SEQ ID NO:139 or in SEQ ID NO:140 or in SEQ ID NO:141 or amino acid sequences being at least 75%, 80%, 85%, 90%, 91%, 92%, 93%, 94%, 95%, 96%, 97%, 98% or 99% identical to the sequence as set forth in SEQ ID NO:142 or in SEQ ID NO:143 or in SEQ ID NO:144 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:142 or in SEQ ID NO:143 or in SEQ ID NO:144 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:139 or in SEQ ID NO:140 or in SEQ ID NO:141.

The term “SLC39A11” refers to the human Solute Carrier Family 39 Member 11 gene (Ensembl: ENSG00000133195), for example, to the sequence as defined in NCBI Reference Sequence NM_139177 or in NCBI Reference Sequence NM_001352692, specifically, to the nucleotide sequence as set forth in SEQ ID NO:145 or in SEQ ID NO:146, which correspond to the sequences of the above indicated NCBI Reference Sequences of the SLC39A11 transcript, and also relates to the corresponding amino acid sequence for example as set forth in SEQ ID NO:147 or in SEQ ID NO:148, which correspond to the protein sequences defined in NCBI Protein Accession Reference Sequence NP_631916 and in NCBI Protein Accession Reference Sequence NP_001339621 encoding the SLC39A11 polypeptide.

The term “SLC39A11” also comprises nucleotide sequences showing a high degree of homology to SLC39A11, e.g., nucleic acid sequences being at least 75%, 80%, 85%, 90%, 91%, 92%, 93%, 94%, 95%, 96%, 97%, 98% or 99% identical to the sequence as set forth in SEQ ID NO:145 or in SEQ ID NO:146 or amino acid sequences being at least 75%, 80%, 85%, 90%, 91%, 92%, 93%, 94%, 95%, 96%, 97%, 98% or 99% identical to the sequence as set forth in SEQ ID NO:147 or in SEQ ID NO:148 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:147 or in SEQ ID NO:148 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:145 or in SEQ ID NO:146.

The term “TDRD1” refers to the human Tudor Domain Containing 1 gene (Ensembl: ENSG00000095627), for example, to the sequence as defined in NCBI Reference Sequence NM_198795, specifically, to the nucleotide sequence as set forth in SEQ ID NO:149, which corresponds to the sequence of the above indicated NCBI Reference Sequence of the TDRD 1 transcript, and also relates to the corresponding amino acid sequence for example as set forth in SEQ ID NO:150, which corresponds to the protein sequence defined in NCBI Protein Accession Reference Sequence NP_942090 encoding the TDRD1 polypeptide.

The term “TDRD1” also comprises nucleotide sequences showing a high degree of homology to TDRD1, e.g., nucleic acid sequences being at least 75%, 80%, 85%, 90%, 91%, 92%, 93%, 94%, 95%, 96%, 97%, 98% or 99% identical to the sequence as set forth in SEQ ID NO:149 or amino acid sequences being at least 75%, 80%, 85%, 90%, 91%, 92%, 93%, 94%, 95%, 96%, 97%, 98% or 99% identical to the sequence as set forth in SEQ ID NO:150 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:150 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:149.

The term “TLR8” refers to the Toll Like Receptor 8 gene (Ensembl: ENSG00000101916), for example, to the sequence as defined in NCBI Reference Sequence NM_138636 or in NCBI Reference Sequence NM_016610, specifically, to the nucleotide sequences as set forth in SEQ ID NO:151 or in SEQ ID NO:152, which correspond to the sequences of the above indicated NCBI Reference Sequences of the TLR8 transcript, and also relates to the corresponding amino acid sequences for example as set forth in SEQ ID NO:153 or in SEQ ID NO:154, which corresponds to the protein sequences defined in NCBI Protein Accession Reference Sequence NP_619542 and in NCBI Protein Accession Reference Sequence NP_057694 encoding the TLR8 polypeptide.

The term “TLR8” also comprises nucleotide sequences showing a high degree of homology to TLR8, e.g., nucleic acid sequences being at least 75%, 80%, 85%, 90%, 91%, 92%, 93%, 94%, 95%, 96%, 97%, 98% or 99% identical to the sequence as set forth in SEQ ID NO:151 or in SEQ ID NO:152 or amino acid sequences being at least 75%, 80%, 85%, 90%, 91%, 92%, 93%, 94%, 95%, 96%, 97%, 98% or 99% identical to the sequence as set forth in SEQ ID NO:153 or in SEQ ID NO:154 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:153 or in SEQ ID NO:154 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:151 or in SEQ ID NO:152.

The term “VWA2” refers to the human Von Willebrand Factor A Domain Containing 2 gene (Ensembl: ENSG00000165816), for example, to the sequence as defined in NCBI Reference Sequence NM_001320804, specifically, to the nucleotide sequence as set forth in SEQ ID NO:155, which corresponds to the sequence of the above indicated NCBI Reference Sequence of the VWA2 transcript, and also relates to the corresponding amino acid sequence for example as set forth in SEQ ID NO:156, which corresponds to the protein sequence defined in NCBI Protein Accession Reference Sequence NP_001307733 encoding the VWA2 polypeptide.

The term “VWA2” also comprises nucleotide sequences showing a high degree of homology to VWA2, e.g., nucleic acid sequences being at least 75%, 80%, 85%, 90%, 91%, 92%, 93%, 94%, 95%, 96%, 97%, 98% or 99% identical to the sequence as set forth in SEQ ID NO:155 or amino acid sequences being at least 75%, 80%, 85%, 90%, 91%, 92%, 93%, 94%, 95%, 96%, 97%, 98% or 99% identical to the sequence as set forth in SEQ ID NO:156 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:156 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:155.

The term “ZAP70” refers to the Zeta Chain Of T-Cell Receptor Associated Protein Kinase 70 gene (Ensembl: ENSG00000115085), for example, to the sequence as defined in NCBI Reference Sequence NM_001079 or in NCBI Reference Sequence NM_207519, specifically, to the nucleotide sequences as set forth in SEQ ID NO:157 or in SEQ ID NO:158, which correspond to the sequences of the above indicated NCBI Reference Sequences of the ZAP70 transcript, and also relates to the corresponding amino acid sequence for example as set forth in SEQ ID NO:159 or in SEQ ID NO:160, which corresponds to the protein sequences defined in NCBI Protein Accession Reference Sequence NP_001070 and in NCBI Protein Accession Reference Sequence NP_997402 encoding the ZAP70 polypeptide.

The term “ZAP70” also comprises nucleotide sequences showing a high degree of homology to ZAP70, e.g., nucleic acid sequences being at least 75%, 80%, 85%, 90%, 91%, 92%, 93%, 94%, 95%, 96%, 97%, 98% or 99% identical to the sequence as set forth in SEQ ID NO:157 or in SEQ ID NO:158or amino acid sequences being at least 75%, 80%, 85%, 90%, 91%, 92%, 93%, 94%, 95%, 96%, 97%, 98% or 99% identical to the sequence as set forth in SEQ ID NO:159 or in SEQ ID NO:160 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:159 or in SEQ ID NO:160 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:157 or in SEQ ID NO:158.

The term “ZBP1” refers to the Z-DNA Binding Protein 1 gene (Ensembl: ENSG00000124256), for example, to the sequence as defined in NCBI Reference Sequence NM_030776 or in NCBI Reference Sequence NM_001160418 or in NCBI Reference Sequence NM_001160419, specifically, to the nucleotide sequence as set forth in SEQ ID NO:161 or in SEQ ID NO:162 or in SEQ ID NO:163, which corresponds to the sequence of the above indicated NCBI Reference Sequence of the ZBP1 transcript, and also relates to the corresponding amino acid sequence for example as set forth in SEQ ID NO:164 or in SEQ ID NO:165 or in SEQ ID NO:166, which corresponds to the protein sequences defined in NCBI Protein Accession Reference Sequence NP_110403 and in NCBI Protein Accession Reference Sequence NP_001153890 and in NCBI Protein Accession Reference Sequence NP_001153891 encoding the ZBP1 polypeptide.

The term “ZBP1” also comprises nucleotide sequences showing a high degree of homology to ZBP1, e.g., nucleic acid sequences being at least 75%, 80%, 85%, 90%, 91%, 92%, 93%, 94%, 95%, 96%, 97%, 98% or 99% identical to the sequence as set forth in SEQ ID NO:161 or in SEQ ID NO:162 or in SEQ ID NO:163 or amino acid sequences being at least 75%, 80%, 85%, 90%, 91%, 92%, 93%, 94%, 95%, 96%, 97%, 98% or 99% identical to the sequence as set forth in SEQ ID NO:164 or in SEQ ID NO:165 or in SEQ ID NO:166or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:164 or in SEQ ID NO:165 or in SEQ ID NO:166 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:161 or in SEQ ID NO:162 or in SEQ ID NO:163.

The term “biological sample” or “sample obtained from a subject” refers to any biological material obtained via suitable methods known to the person skilled in the art from a subject, e.g., a prostate cancer patient. The term “prostate cancer subject” refers to a person having or suspected of having prostate cancer.

It was further demonstrated that use of the full model (i.e. using all 8 PDE4D7 correlated genes, all 14 immune defense response genes and all 17 T-Cell receptor signaling genes) is not essential to obtain a significant predictive effect, and that significant results can already be obtained with a random selection of one of the PDE4D7 correlated genes combined with one randomly selected immune defense response gene and one randomly selected T-Cell receptor signaling gene. The examples and FIGS. 15-32 demonstrate that a random selection of one of the PDE4D7 correlated genes combined with one randomly selected immune defense response gene and one randomly selected T-Cell receptor signaling gene, that were selected from the full set of genes suffice to make a significant prediction. These random selections make it plausible that any selection of one gene from each group allows for a significant prediction of the response of a prostate cancer subject to radiotherapy.

The biological sample used may be collected in a clinically acceptable manner, e.g., in a way that nucleic acids (in particular RNA) or proteins are preserved.

The biological sample(s) may include body tissue and/or a fluid, such as, but not limited to, blood, sweat, saliva, and urine. Furthermore, the biological sample may contain a cell extract derived from or a cell population including an epithelial cell, such as a cancerous epithelial cell or an epithelial cell derived from tissue suspected to be cancerous. The biological sample may contain a cell population derived from a glandular tissue, e.g., the sample may be derived from the prostate of a male subject. Additionally, cells may be purified from obtained body tissues and fluids if necessary, and then used as the biological sample. In some realizations, the sample may be a tissue sample, a urine sample, a urine sediment sample, a blood sample, a saliva sample, a semen sample, a sample including circulating tumour cells, extracellular vesicles, a sample containing prostate secreted exosomes, or cell lines or cancer cell line.

In one particular realization, biopsy or resections samples may be obtained and/or used. Such samples may include cells or cell lysates.

It is also conceivable that the content of a biological sample is submitted to an enrichment step. For instance, a sample may be contacted with ligands specific for the cell membrane or organelles of certain cell types, e.g., prostate cells, functionalized for example with magnetic particles. The material concentrated by the magnetic particles may subsequently be used for detection and analysis steps as described herein above or below.

Furthermore, cells, e.g., tumour cells, may be enriched via filtration processes of fluid or liquid samples, e.g., blood, urine, etc. Such filtration processes may also be combined with enrichment steps based on ligand specific interactions as described herein above.

The term “prostate cancer” refers to a cancer of the prostate gland in the male reproductive system, which occurs when cells of the prostate mutate and begin to multiply out of control. Typically, prostate cancer is linked to an elevated level of prostate-specific antigen (PSA). In one embodiment of the present invention the term “prostate cancer” relates to a cancer showing PSA levels above 3.0. In another embodiment the term relates to cancer showing PSA levels above 2.0. The term “PSA level” refers to the concentration of PSA in the blood in ng/ml.

The term “non-progressive prostate cancer state” means that a sample of an individual does not show parameter values indicating “biochemical recurrence” and/or “clinical recurrence” and/or “metastases” and/or “castration-resistant disease” and/or “prostate cancer or disease specific death”.

The term “progressive prostate cancer state” means that a sample of an individual shows parameter values indicating “biochemical recurrence” and/or “clinical recurrence” and/or “metastases” and/or “castration-resistant disease” and/or “prostate cancer or disease specific death”.

The term “biochemical recurrence” generally refers to recurrent biological values of increased PSA indicating the presence of prostate cancer cells in a sample. However, it is also possible to use other markers that can be used in the detection of the presence or that rise suspicion of such presence.

The term “clinical recurrence” refers to the presence of clinical signs indicating the presence of tumour cells as measured, for example using in vivo imaging.

The term “metastases” refers to the presence of metastatic disease in organs other than the prostate.

The term “castration-resistant disease” refers to the presence of hormone-insensitive prostate cancer; i.e., a cancer in the prostate that does not any longer respond to androgen deprivation therapy (ADT).

The term “prostate cancer specific death or disease specific death” refers to death of a patient from his prostate cancer.

It is preferred that:
the one or more immune defense response genes comprise three or more, preferably, six or more, more preferably, nine or more, most preferably, all of the immune defense genes, and/or
the one or more T-Cell receptor signaling genes comprise three or more, preferably, six or more, more preferably, nine or more, most preferably, all of the T-Cell receptor signaling genes, and/or
the one or more PDE4D7 correlated genes comprise three or more, preferably, six or more, most preferably, all of the PDE4D7 correlated genes.
   It is preferred that the determining of the prediction of the therapy response or of the personalization of the therapy comprises:
combining the first gene expression profiles for two or more, for example, 2, 3, 4, 5, 6, 7, 8, 9, 10, 11, 12, 13 or all, of the immune defense response genes with a regression function that had been derived from a population of prostate cancer subjects, and/or
combining the second gene expression profiles for two or more, for example, 2, 3, 4, 5, 6, 7, 8, 9, 10, 11, 12, 13, 14, 15, 16 or all, of the T-Cell receptor signaling genes with a regression function that had been derived from a population of prostate cancer subjects, and/or
combining the third gene expression profiles for two or more, for example, 2, 3, 4, 5, 6, 7 or all, of the PDE4D7 correlated genes with a regression function that had been derived from a population of prostate cancer subjects.

Cox proportional-hazards regression allows analyzing the effect of several risk factors on time to a tested event like survival. Thereby, the risk factors maybe dichotomous or discrete variables like a risk score or a clinical stage but may also be a continuous variable like a biomarker measurement or gene expression values. The probability of the endpoint (e.g., death or disease recurrence) is called the hazard. Next to the information on whether or not the tested endpoint was reached by e.g. subject in a patient cohort (e.g., patient did die or not) also the time to the endpoint is considered in the regression analysis. The hazard is modeled as: H(t)=H₀(t)·exp(w₁·V₁+w₂·V₂+w₃·V₃+ . . . ), where V₁, V₂, V₃ . . . are predictor variables and H₀(t) is the baseline hazard while H(t) is the hazard at any time t. The hazard ratio (or the risk to reach the event) is represented by Ln[H(t)/H₀(t)]=w₁·V₁+w₂·V₂+w₃·V₃+ . . . , where the coefficients or weights w₁, w₂, w₃ . . . are estimated by the Cox regression analysis and can be interpreted in a similar manner as for logistic regression analysis.

In one particular realization, the combination of the first gene expression profiles for the two or more, for example, 2, 3, 4, 5, 6, 7, 8, 9, 10, 11, 12, 13 or all, of the immune defense response genes with a regression function is determined as follows:

IDR_model:

(w₁·AIM2)+(w₂·APOBEC3A)+(w₃·CIAO1)+(w₄·DDX58)+(w₅·DHX9)+(w₆·IFI16)+(w₇·IFIH1)+(w₈·IFIT1)+(w₉·IFIT3)+(w₁₀·LRRFIP1)+(w₁₁·MYD88)+(w₁₂·OAS1)+(w₁₃·TLR8)+(w₁₄·ZBP1) (2)

where w₁ to w₁₄ are weights and AIM2, APOBEC3A, CIAO1, DDX58, DHX9, IFI16, IFIH1, IFIT1, IFIT3, LRRFIP1, MYD88, OAS1, TLR8, and ZBP1 are the expression levels of the immune defense response genes.

In one example, w₁ may be about −0.8 to 0.2, such as −0.313, w₂ may be about −0.7 to 0.3, such as −0.1417, w₃ may be about −0.4 to 0.6, such as 0.1008, w₄ may be about −0.5 to 0.5, such as −0.07478, w₅ may be about −0.2 to 0.8, such as 0.277, w₆ may be about −0.6 to 0.4, such as −0.07944, w₇ may be about −0.2 to 0.8, such as 0.3036, w₈ may be about −0.6 to 0.4, such as −0.09188, w₉ may be about −0.3 to 0.7, such as 0.1661, w₁₀ may be about −1.2 to 0.2, such as −0.7105, w₁₁ may be about −0.3 to 0.7, such as 0.1615, w₁₂ may be about −0.6 to 0.4, such as −0.07468, w₁₃ may be about −0.6 to 0.4, such as −0.06677, and w₁₄ may be about −0.7 to 0.3, such as −0.155.

In one particular realization, the combination of the second gene expression profiles for the two or more, for example, 2, 3, 4, 5, 6, 7, 8, 9, 10, 11, 12, 13, 14, 15, 16 or all, of the T-Cell receptor signaling genes with a regression function is determined as follows:

TCR_SIGNALING_model:

(w₁₅·C2)+(w₁₆·CD247)+(w₁₇·CD28)+(w₁₈·CD3E)+(w₁₉·CD3G)+(w₂₀·CD4)+(w₂₁·CSK)+(w₂₂·EZR)+(w₂₃·FYN)+(w₂₄·LAT)+(w₂₅·LCK)+(w₂₆·PAG1)+(w₂₇·PDE4D)+(w₂₈·PRKACA)+(w₂₉·PRKACB)+(w₃₀·PTPRC)+(w₃₁·ZAP70) (3)

where w₁₅ to w₃₁ are weights and CD2, CD247, CD28, CD3E, CD3G, CD4, CSK, EZR, FYN, LAT, LCK, PAG1, PDE4D, PRKACA, PRKACB, PTPRC, and ZAP70 are the expression levels of the T-Cell receptor signaling genes.

In one example, w₁₅ may be about −0.1 to 0.9, such as 0.4137, w₁₆ may be about −0.6 to 0.4, such as −0.1323, w₁₇ may be about −0.1 to 0.9, such as 0.3695, w₁₈ may be about −0.8 to 0.2, such as −0.267, w₁₉ may be about −0.7 to 0.3, such as −0.1984, w₂₀ may be about −0.2 to 0.8, such as 0.3018, w₂₁ may be about 0.1 to 1.1, such as 0.6169, w₂₂ may be about −0.8 to 0.2, such as −0.2789, w₂₃ may be about −0.7 to 0.3, such as −0.1842, w₂₄ may be about 0.5 to 1.5, such as 0.4672, w₂₅ may be about −0.5 to 0.5, such as −0.07028, w₂₆ may be about −0.2 to 0.8, such as 0.3278, w₂₇ may be about −1.3 to −0.3, such as −0.8253, w₂₈ may be about 0.1 to 1.1, such as 0.6212, w₂₉ may be about −0.9 to 0.1, such as −0.4462, w₃₀ may be about −0.9 to 0.1, such as −0.4622, and w₃₁ may be about −0.1 to 0.9, such as −0.3702.

In one particular realization, the combination of the third gene expression profiles for the two or more, for example, 2, 3, 4, 5, 6, 7 or all, of the PDE4D7 correlated genes with a regression function is determined as follows:

PDE4D7_CORR_model:

(w₃₂·ABCC5)+(w₃₃·CUX2)+(w₃₄·KIAA1549)+(w₃₅·PDE4D)+(w₃₆·RAP1GAP2)+(w₃₇·SLC39A11)+(w₃₈·TDRD1)+(w₃₉·VWA2) (4)

where w₃₂ to w₃₉ are weights and ABCC5, CUX2, KIAA1549, PDE4D, RAP1GAP2, SLC39A11, TDRD1, and VWA2 are the expression levels of the PDE4D7 correlated genes.

In one example, w₃₂ may be about −0.1 to 0.9, such as 0.368, w₃₃ may be about −0.3 to −1.3, such as −0.7675, w₃₄ may be about −0.1 to 0.9, such as 0.4108, w₃₅ may be about −0.1 to 0.9, such as 0.4007, w₃₆ may be about −1.2 to −0.2, such as −0.679, w₃₇ may be about 0.0 to 0.1, such as 0.5433, w₃₈ may be about 0.1 to 1.1, such as 0.6366, and w₃₉ may be about −1.0 to 0.0, such as −0.4749.

It is further preferred that the determining of the prediction of the therapy response or of the personalization of the therapy further comprises combining the combination of the first gene expression profiles, the combination of the second gene expression profiles, and the combination of the third gene expression profiles with a regression function that had been derived from a population of prostate cancer subjects.

In one particular realization, the prediction of the therapy response or the personalization of the therapy is determined as follows:

PCAI_model:

(w₄₀·IDR_model)+(w₄₁TCR_SIGNALING_model)+(w₄₂·PDE4D7_CORR_model) (5)

where w₄₀ to w₄₂ are weights, IDR_model is the above-described regression model based on the expression profiles for the two or more, for example, 2, 3, 4, 6, 7, 8, 9, 10, 11, 12, 13 or all, of the immune defense response genes, TCR_SIGNALING_model is the above-described regression model based on the expression profiles for the two or more, for example, 2, 3, 4, 6, 7, 8, 9, 10, 11, 12, 13, 14, 15, 16 or all, of the T-Cell receptor signaling genes, and PDE4D7_CORR_model is the above-described regression model based on the expression profiles for the two or more, for example, 2, 3, 4, 6, 7 or all, of the PDE4D7 correlated genes.

In one example, w₄₀ may be about 0.2 to 1.2, such as 0.674, w₄₁ may be about 0.0 to 1.0, such as 0.5474, and w₄₂ may be about 0.1 to 1.1, such as 0.6372.

The prediction of the therapy response may also be classified or categorized into one of at least two risk groups, based on the value of the prediction of the therapy response. For example, there may be two risk groups, or three risk groups, or four risk groups, or more than four predefined risk groups. Each risk group covers a respective range of (non-overlapping) values of the prediction of the therapy response. For example, a risk group may indicate a probability of occurrence of a specific clinical event from 0 to <0.1 or from 0.1 to <0.25 or from 0.25 to <0.5 or from 0.5 to 1.0 or the like.

It is further preferred that the determining of the prediction of the therapy response or of the personalization of the therapy is further based on one or more clinical parameters obtained from the subject.

As mentioned above, various measures based on clinical parameters have been investigated. By further basing the prediction of the therapy response or the personalization of the therapy on such clinical parameter(s), it can be possible to further improve the prediction.

It is preferred that the clinical parameters comprise one or more of: (i) a prostate-specific antigen (PSA) level; (ii) a pathologic Gleason score (pGS); (iii) a clinical tumour stage; (iv) a pathological Gleason grade group (pGGG); (v) a pathological stage; (vi) one or more pathological variables, for example, a status of surgical margins and/or a lymph node invasion and/or an extra-prostatic growth and/or a seminal vesicle invasion; (vii) CAPRA; (viii) CAPRA-S; (ix) EAU-BCR risk groups, and; (x) another clinical risk score.

It is further preferred that the determining of the prediction of the therapy response or the personalization of the therapy comprises combining one or more of: (i) the first gene expression profile(s) for the one or more immune defense response genes; (ii) the second gene expression profile(s) for the one or more T-Cell receptor signaling genes; (iii) the third gene expression profile(s) for the one or more PDE4D7 correlated genes, and; (iv) the combination of the first gene expression profiles, the combination of the second gene expression profiles, and the combination of the third gene expression profiles, and the one or more clinical parameters obtained from the subject with a regression function that had been derived from a population of prostate cancer subjects.

In one particular realization, the prediction of the therapy response is determined as follows:

PCAI&Clinical_model:

(w₄₃·PCAI_model)+(w₄₄·EAU_BCR) (6)

where w₄₃ and w₄₄ are weights, PCAI_model is the above-described regression model based on the expression profiles for the two or more, for example, 2, 3, 4, 6, 7, 8, 9, 10, 11, 12, 13 or all, of the immune defense response genes, the expression profiles for the two or more, for example, 2, 3, 4, 6, 7, 8, 9, 10, 11, 12, 13, 14, 15, 16 or all, of the T-Cell receptor signaling genes, and the expression profiles for the two or more, for example, 2, 3, 4, 6, 7 or all, of the PDE4D7 correlated genes, and EAU_BCR is the EAU-BCR risk group (see Tilki D. et al., “External validation of the European Association of Urology Biochemical Recurrence Risk groups to predict metastasis and mortality after radical prostatectomy in a European cohort”, Eur Urol, Vol. 75, No. 6, pages 896-900, 2019).

In one example, w₄₃ may be about 0.4 to 1.4, such as 0.8887, and w₄₄ may be about 1.1 to 2.1, such as 1.6085.

It is preferred that the biological sample is obtained from the subject before the start of the therapy. The gene expression profile(s) may be determined in the form of mRNA or protein in tissue of prostate cancer. Alternatively, if the genes are present in a soluble form, the gene expression profile(s) may be determined in blood.

It is further preferred that the therapy is radical radiotherapy, salvage radiotherapy (SRT), salvage androgen deprivation therapy (SADT), or cytotoxic chemotherapy (CTX).

It is preferred that the therapy is radiotherapy, wherein the prediction of the therapy response is negative or positive for the effectiveness of the therapy, wherein a therapy is recommended based on the prediction and, if the prediction is negative, the recommended therapy comprises one or more of: (i) radiotherapy provided earlier than is the standard; (ii) radiotherapy with an increased radiation dose; (iii) an adjuvant therapy, such as androgen deprivation therapy; and iv) an alternative therapy that is not a radiation therapy.

In a further aspect of the present invention, an apparatus for predicting a response of a prostate cancer subject to therapy or for personalizing therapy of a prostate cancer subject is presented, comprising:
an input adapted to receive data indicative of a first gene expression profile for each of one or more, for example, 1, 2, 3, 4, 5, 6, 7, 8, 9, 10, 11, 12, 13 or all, immune defense response genes selected from the group consisting of: AIM2, APOBEC3A, CIAO1, DDX58, DHX9, IFI16, IFIH1, IFIT1, IFIT3, LRRFIP1, MYD88, OAS1, TLR8, and ZBP1, said first gene expression profile(s) being determined in a biological sample obtained from the subject, of a second gene expression profile for each of one or more, for example, 1, 2, 3, 4, 5, 6, 7, 8, 9, 10, 11, 12, 13, 14, 15, 16 or all, T-Cell receptor signaling genes selected from the group consisting of: CD2, CD247, CD28, CD3E, CD3G, CD4, CSK, EZR, FYN, LAT, LCK, PAG1, PDE4D, PRKACA, PRKACB, PTPRC, and ZAP70, said second gene expression profile(s) being determined in a biological sample obtained from the subject, and of a third gene expression profile for each of one or more, for example, 1, 2, 3, 4, 5, 6, 7 or all, PDE4D7 correlated genes selected from the group consisting of: ABCC5, CUX2, KIAA1549, PDE4D, RAP1GAP2, SLC39A11, TDRD1, and VWA2, said third gene expression profile(s) being determined in a biological sample obtained from the subject,
a processor adapted to determine the prediction of the therapy response or the personalization of the therapy based on the first, second, and third gene expression profile(s), and
optionally, a providing unit adapted to provide the prediction or the personalization or a therapy recommendation based on the prediction or the personalization to a medical caregiver or the subject.

In a further aspect of the present invention, a computer program product is presented comprising instructions which, when the program is executed by a computer, cause the computer to carry out a method comprising:
receiving data indicative of a first gene expression profile for each of one or more, for example, 1, 2, 3, 4, 5, 6, 7, 8, 9, 10, 11, 12, 13 or all, immune defense response genes selected from the group consisting of: AIM2, APOBEC3A, CIAO1, DDX58, DHX9, IFI16, IFIH1, IFIT1, IFIT3, LRRFIP1, MYD88, OAS1, TLR8, and ZBP1, said first gene expression profile(s) being determined in a biological sample obtained from the subject, of a second gene expression profile for each of one or more, for example, 1, 2, 3, 4, 5, 6, 7, 8, 9, 10, 11, 12, 13, 14, 15, 16 or all, T-Cell receptor signaling genes selected from the group consisting of: CD2, CD247, CD28, CD3E, CD3G, CD4, CSK, EZR, FYN, LAT, LCK, PAG1, PDE4D, PRKACA, PRKACB, PTPRC, and ZAP70, said second gene expression profile(s) being determined in a biological sample obtained from the subject, and of a third gene expression profile for each of one or more, for example, 1, 2, 3, 4, 5, 6, 7 or all, PDE4D7 correlated genes selected from the group consisting of: ABCC5, CUX2, KIAA1549, PDE4D, RAP1GAP2, SLC39A11, TDRD1, and VWA2, said third gene expression profile(s) being determined in a biological sample obtained from the subject,
determining a prediction of a response of a prostate cancer subject to therapy or a personalization of therapy of a prostate cancer subject based on the first, second, and third gene expression profile(s), and
optionally, providing the prediction or the personalization or a therapy recommendation based on the prediction or the personalization to a medical caregiver or the subject.

In a further aspect of the present invention, a diagnostic kit is presented, comprising:
at least one primer and/or probe for determining a first gene expression profile for each of one or more, for example, 1, 2, 3, 4, 5, 6, 7, 8, 9, 10, 11, 12, 13 or all, immune defense response genes selected from the group consisting of: AIM2, APOBEC3A, CIAO1, DDX58, DHX9, IFI16, IFIH1, IFIT1, IFIT3, LRRFIP1, MYD88, OAS1, TLR8, and ZBP1, in a biological sample obtained from the subject,
at least one primer and/or probe for determining a second gene expression profile for each of one or more, for example, 1, 2, 3, 4, 5, 6, 7, 8, 9, 10, 11, 12, 13, 14, 15, 16 or all, T-Cell receptor signaling genes selected from the group consisting of: CD2, CD247, CD28, CD3E, CD3G, CD4, CSK, EZR, FYN, LAT, LCK, PAG1, PDE4D, PRKACA, PRKACB, PTPRC, and ZAP70, in a biological sample obtained from the subject,
at least one primer and/or probe for determining a gene expression profile for each of one or more, for example, 1, 2, 3, 4, 5, 6, 7 or all, PDE4D7 correlated genes selected from the group consisting of: ABCC5, CUX2, KIAA1549, PDE4D, RAP1GAP2, SLC39A11, TDRD1, and VWA2, in a biological sample obtained from the subject, and
optionally, an apparatus as defined in claim 11 or a computer program product as defined in claim 12.

In a further aspect of the present invention, a use of the kit as defined in claim 13 is presented.

It is preferred that the use as defined in claim 14 is in a method of predicting a response of a prostate cancer subject to therapy or of personalizing therapy of a prostate cancer subject.

In a further aspect of the present invention, a method is presented, comprising:
receiving one or more biological sample(s) obtained from a prostate cancer subject,
using the kit as defined in claim 13 to determine a first gene expression profile for each of one or more, for example, 1, 2, 3, 4, 5, 6, 7, 8, 9, 10, 11, 12, 13 or all, immune defense response genes selected from the group consisting of: AIM2, APOBEC3A, CIAO1, DDX58, DHX9, IFI16, IFIH1, IFIT1, IFIT3, LRRFIP1, MYD88, OAS1, TLR8, and ZBP1, in a biological sample obtained from the subject,
using the kit as defined in claim 13 to determine a second gene expression profile for each of one or more, for example, 1, 2, 3, 4, 5, 6, 7, 8, 9, 10, 11, 12, 13, 14, 15, 16 or all, T-Cell receptor signaling genes selected from the group consisting of: CD2, CD247, CD28, CD3E, CD3G, CD4, CSK, EZR, FYN, LAT, LCK, PAG1, PDE4D, PRKACA, PRKACB, PTPRC, and ZAP70, in a biological sample obtained from the subject,
using the kit as defined in claim 13 to determine a third gene expression profile for each of one or more, for example, 1, 2, 3, 4, 5, 6, 7 or all, PDE4D7 correlated genes selected from the group consisting of: ABCC5, CUX2, KIAA1549, PDE4D, RAP1GAP2, SLC39A11, TDRD1, and VWA2, in a biological sample obtained from the subject.

In a further aspect of the present invention, a use of a first gene expression profile for each of one or more, for example, 1, 2, 3, 4, 5, 6, 7, 8, 9, 10, 11, 12, 13 or all, immune defense response genes selected from the group consisting of: AIM2, APOBEC3A, CIAO1, DDX58, DHX9, IFI16, IFIH1, IFIT1, IFIT3, LRRFIP1, MYD88, OAS1, TLR8, and ZBP1, of a second gene expression profile for each of one or more, for example, 1, 2, 3, 4, 5, 6, 7, 8, 9, 10, 11, 12, 13, 14, 15, 16 or all, T-Cell receptor signaling genes selected from the group consisting of: CD2, CD247, CD28, CD3E, CD3G, CD4, CSK, EZR, FYN, LAT, LCK, PAG1, PDE4D, PRKACA, PRKACB, PTPRC, and ZAP70, and of a third gene expression profile for each of one or more, for example, 1, 2, 3, 4, 5, 6, 7 or all, PDE4D7 correlated genes selected from the group consisting of: ABCC5, CUX2, KIAA1549, PDE4D, RAP1GAP2, SLC39A11, TDRD1, and VWA2, in a method of predicting a response of a prostate cancer subject to therapy or of personalizing therapy of a prostate cancer subject is presented, comprising:
determining the prediction of the therapy response or the personalization of the therapy based on the first, second, and third gene expression profile(s), and
optionally, providing the prediction or the personalization or a therapy recommendation based on the prediction or the personalization to a medical caregiver or the subject.

It shall be understood that the method of claim 1, the apparatus of claim 11, the computer program product of claim 12, the diagnostic kit of claim 13, the use of the diagnostic kit of claim 14, the method of claim 16, and the use of first, second, and third gene expression profile(s) of claim 17 have similar and/or identical preferred embodiments, in particular, as defined in the dependent claims.

It shall be understood that a preferred embodiment of the present invention can also be any combination of the dependent claims or above embodiments with the respective independent claim.

These and other aspects of the invention will be apparent from and elucidated with reference to the embodiments described hereinafter.

### BRIEF DESCRIPTION OF THE DRAWINGS

In the following drawings:

FIG. 1 shows schematically and exemplarily a flowchart of an embodiment of a method of predicting a response of a prostate cancer subject to therapy or of personalizing therapy of a prostate cancer subject,

FIG. 2 shows a Kaplan-Meier curve of the PCAI_model in a 186 patient cohort (training set used to develop the PCAI_model) with all patients undergoing SRT (salvage radiation treatment) after post-surgical BCR (biochemical recurrence),

FIG. 3 shows a Kaplan-Meier curve of the PCAI_model in a 151 patient cohort (testing set used to validate the PCAI_model as developed on the 186 patient training set) with all patients undergoing SRT (salvage radiation treatment) after post-surgical BCR (biochemical recurrence),

FIG. 4 shows a Kaplan-Meier curve of the PCAI_model in a 151 patient cohort (testing set used to validate the PCAI_model as developed on the 186 patient training set) with all patients undergoing SRT (salvage radiation treatment) after post-surgical BCR (biochemical recurrence),

FIG. 5 shows a Kaplan-Meier curve of the PCAI_model in a 125 patient cohort (training set used to develop the PCAI_model) with all patients undergoing SADT (salvage androgen deprivation therapy) after post-surgical BCR (biochemical recurrence),

FIG. 6 shows a Kaplan-Meier curve of the PCAI_model in a 66 patient cohort (testing set used to validate the PCAI_model as developed on the 186 patient training set) with all patients undergoing SADT (salvage androgen deprivation therapy) after post-surgical BCR (biochemical recurrence),

FIG. 7 shows a Kaplan-Meier curve of the PCAI_model in a 66 patient cohort (testing set used to validate the PCAI_model as developed on the 186 patient training set) with all patients undergoing SADT (salvage androgen deprivation therapy) after post-surgical BCR (biochemical recurrence),

FIG. 8 shows a Kaplan-Meier curve of the PCAI_model in a 20 patient cohort (training set used to develop the PCAI_model) with all patients undergoing CTX (chemotherapy) after post-surgical BCR (biochemical recurrence),

FIG. 9 shows a Kaplan-Meier curve of the PCAI&Clinical_model in a 159 patient cohort (training set used to develop the PCAI&Clinical_model) with all patients undergoing SRT (salvage radiation treatment) after post-surgical BCR (biochemical recurrence),

FIG. 10 shows a Kaplan-Meier curve of the PCAI&Clinical_model in a 151 patient cohort (testing set used to validate the PCAI&Clinical_model as developed on the 186 patient training set) with all patients undergoing SRT (salvage radiation treatment) after post-surgical BCR (biochemical recurrence),

FIG. 11 shows a Kaplan-Meier curve of the PCAI&Clinical_model in a 151 patient cohort (testing set used to validate the PCAI&Clinical_model as developed on the 186 patient training set) with all patients undergoing SRT (salvage radiation treatment) after post-surgical BCR (biochemical recurrence),

FIG. 12 shows a Kaplan-Meier curve of the PCAI&Clinical_model in a 91 patient cohort (training set used to develop the PCAI_model) with all patients undergoing SADT (salvage androgen deprivation therapy) after post-surgical BCR (biochemical recurrence),

FIG. 13 shows a Kaplan-Meier curve of the PCAI&Clinical_model in a 66 patient cohort (testing set used to validate the PCAI_model as developed on the 186 patient training set) with all patients undergoing SADT (salvage androgen deprivation therapy) after post-surgical BCR (biochemical recurrence), and

FIG. 14 shows a Kaplan-Meier curve of the PCAI&Clinical_model in a 66 patient cohort (testing set used to validate the PCAI_model as developed on the 186 patient training set) with all patients undergoing SADT (salvage androgen deprivation therapy) after post-surgical BCR (biochemical recurrence).

FIG. 15 shows a Kaplan-Meier curve of a model comprising a random combination of genes comprising one immune defense response gene, one TCR signaling gene and one PDE4D7 correlated gene (PCAI-3.1_model) in a 185 patients cohort. The clinical endpoint that was tested was the time to prostate cancer specific death (PCa Death) after the start of salvage radiation therapy (SRT) due to post-surgical disease recurrence.

FIG. 16 shows a Kaplan-Meier curve of another model comprising a random combination of genes comprising one immune defense response gene, one TCR signaling gene and one PDE4D7 correlated gene (PCAI-3.2_model) in a 185 patients cohort. The clinical endpoint that was tested was the time to prostate cancer specific death (PCa Death) after the start of salvage radiation therapy (SRT) due to post-surgical disease recurrence.

FIG. 17 shows a Kaplan-Meier curve of a reference model comprising a random combination of genes comprising two TCR signaling gene and one PDE4D7 correlated gene (PCAI-3.3_model) in a 185 patients cohort. The clinical endpoint that was tested was the time to prostate cancer specific death (PCa Death) after the start of salvage radiation therapy (SRT) due to post-surgical disease recurrence.

FIG. 18 shows a Kaplan-Meier curve of another model comprising a random combination of genes comprising one immune defense response gene, one TCR signaling gene and one PDE4D7 correlated gene (PCAI-3.4 model) in a 185 patients cohort. The clinical endpoint that was tested was the time to prostate cancer specific death (PCa Death) after the start of salvage radiation therapy (SRT) due to post-surgical disease recurrence.

FIG. 19 shows a Kaplan-Meier curve of another model comprising a random combination of genes comprising one immune defense response gene, one TCR signaling gene and one PDE4D7 correlated gene (PCAI-3.5_model) in a 185 patients cohort. The clinical endpoint that was tested was the time to prostate cancer specific death (PCa Death) after the start of salvage radiation therapy (SRT) due to post-surgical disease recurrence.

FIG. 20 shows a Kaplan-Meier curve of another model comprising a random combination of genes comprising one immune defense response gene, one TCR signaling gene and one PDE4D7 correlated gene (PCAI-3.6_model) in a 185 patients cohort. The clinical endpoint that was tested was the time to prostate cancer specific death (PCa Death) after the start of salvage radiation therapy (SRT) due to post-surgical disease recurrence.

FIG. 21 shows a Kaplan-Meier curve of a model comprising a random combination of genes comprising one immune defense response gene, one TCR signaling gene and one PDE4D7 correlated gene (PCAI-3.1_model) in a 106 patients cohort. The clinical endpoint that was tested was the time to prostate cancer specific death (PCa Death) after the start of salvage androgen deprivation therapy (SADT) due to post-surgical disease recurrence.

FIG. 22 shows a Kaplan-Meier curve of a model comprising a random combination of genes comprising one immune defense response gene, one TCR signaling gene and one PDE4D7 correlated gene (PCAI-3.2_model) in a 106 patients cohort. The clinical endpoint that was tested was the time to prostate cancer specific death (PCa Death) after the start of salvage androgen deprivation therapy (SADT) due to post-surgical disease recurrence.

FIG. 23 shows a Kaplan-Meier curve of a reference model comprising a random combination of genes comprising two TCR signaling gene and one PDE4D7 correlated gene (PCAI-3.3_model) in a 106 patients cohort. The clinical endpoint that was tested was the time to prostate cancer specific death (PCa Death) after the start of salvage androgen deprivation therapy (SADT) due to post-surgical disease recurrence.

FIG. 24 shows a Kaplan-Meier curve of a model comprising a random combination of genes comprising one immune defense response gene, one TCR signaling gene and one PDE4D7 correlated gene (PCAI-3.4_model) in a 106 patients cohort. The clinical endpoint that was tested was the time to prostate cancer specific death (PCa Death) after the start of salvage androgen deprivation therapy (SADT) due to post-surgical disease recurrence.

FIG. 25 shows a Kaplan-Meier curve of a model comprising a random combination of genes comprising one immune defense response gene, one TCR signaling gene and one PDE4D7 correlated gene (PCAI-3.5_model) in a 106 patients cohort. The clinical endpoint that was tested was the time to prostate cancer specific death (PCa Death) after the start of salvage androgen deprivation therapy (SADT) due to post-surgical disease recurrence.

FIG. 26 shows a Kaplan-Meier curve of a model comprising a random combination of genes comprising one immune defense response gene, one TCR signaling gene and one PDE4D7 correlated gene (PCAI-3.6_model) in a 106 patients cohort. The clinical endpoint that was tested was the time to prostate cancer specific death (PCa Death) after the start of salvage androgen deprivation therapy (SADT) due to post-surgical disease recurrence.

FIG. 27 shows a Kaplan-Meier curve of a model comprising a random combination of genes comprising one immune defense response gene, one TCR signaling gene and one PDE4D7 correlated gene (PCAI-3.1_model) in a 571 patients cohort. The clinical endpoint that was tested was the time to prostate cancer specific death (PCa Death) after surgery.

FIG. 28 shows a Kaplan-Meier curve of a model comprising a random combination of genes comprising one immune defense response gene, one TCR signaling gene and one PDE4D7 correlated gene (PCAI-3.2_model) in a 571 patients cohort. The clinical endpoint that was tested was the time to prostate cancer specific death (PCa Death) after surgery.

FIG. 29 shows a Kaplan-Meier curve of a reference model comprising a random combination of genes comprising two TCR signaling gene and one PDE4D7 correlated gene (PCAI-3.3_model) in a 571 patients cohort. The clinical endpoint that was tested was the time to prostate cancer specific death (PCa Death) after surgery.

FIG. 30 shows a Kaplan-Meier curve of a model comprising a random combination of genes comprising one immune defense response gene, one TCR signaling gene and one PDE4D7 correlated gene (PCAI-3.4_model) in a 571 patients cohort. The clinical endpoint that was tested was the time to prostate cancer specific death (PCa Death) after surgery.

FIG. 31 shows a Kaplan-Meier curve of a model comprising a random combination of genes comprising one immune defense response gene, one TCR signaling gene and one PDE4D7 correlated gene (PCAI-3.5_model) in a 571 patients cohort. The clinical endpoint that was tested was the time to prostate cancer specific death (PCa Death) after surgery.

FIG. 32 shows a Kaplan-Meier curve of a model comprising a random combination of genes comprising one immune defense response gene, one TCR signaling gene and one PDE4D7 correlated gene (PCAI-3.6_model) in a 571 patients cohort. The clinical endpoint that was tested was the time to prostate cancer specific death (PCa Death) after surgery.

### DETAILED DESCRIPTION OF EMBODIMENTS

### Overview Of Therapy Response Prediction Or Therapy Personalization

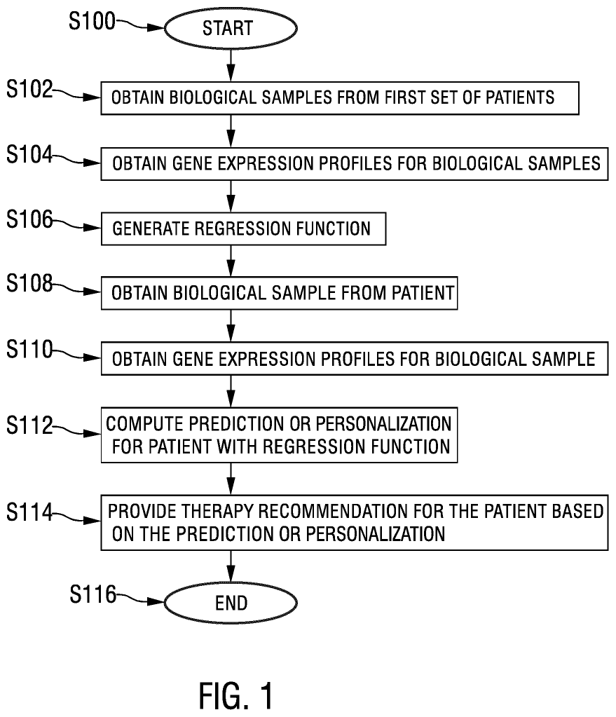

FIG. 1 shows schematically and exemplarily a flowchart of an embodiment of a method of predicting a response of a prostate cancer subject to therapy or of personalizing therapy of a prostate cancer subject.

The method begins at step S100.

At step S102, a biological sample is obtained from each of a first set of patients (subjects) diagnosed with prostate cancer. Preferably, monitoring prostate cancer has been performed for these prostate cancer patients over a period of time, such as at least one year, or at least two years, or about five years, after obtaining the biological sample.

At step S104, a first gene expression profile for each of one or more, for example, 1, 2, 3, 4, 5, 6, 7, 8, 9, 10, 11, 12, 13 or all, immune defense response genes selected from the group consisting of: AIM2, APOBEC3A, CIAO1, DDX58, DHX9, IFI16, IFIH1, IFIT1, IFIT3, LRRFIP1, MYD88, OAS1, TLR8, and ZBP1, a second gene expression profile for each of one or more, for example, 1, 2, 3, 4, 5, 6, 7, 8, 9, 10, 11, 12, 13, 14, 15, 16 or all, T-Cell receptor signaling genes selected from the group consisting of: CD2, CD247, CD28, CD3E, CD3G, CD4, CSK, EZR, FYN, LAT, LCK, PAG1, PDE4D, PRKACA, PRKACB, PTPRC, and ZAP70, and a third gene expression profile for each of two or more, for example, 2, 3, 4, 5, 6, 7 or all, PDE4D7 correlated genes selected from the group consisting of: ABCC5, CUX2, KIAA1549, PDE4D, RAP1GAP2, SLC39A11, TDRD1, and VWA2, is obtained for each of the biological samples obtained from the first set of patients, e.g., by performing RT-qPCR (real-time quantitative PCR) on RNA extracted from each biological sample. The exemplary gene expression profiles include an expression level (e.g., value) for each of the two or more genes which can be normalized using value(s) for each of a set of reference genes, such as B2M, HPRT1, POLR2A, and/or PUM1. In one realization, the gene expression level for each of the two or more genes of the first gene expression profiles, the second gene expression profiles, and the third gene expression profiles is normalized with respect to one or more reference genes selected from the group consisting of ACTB, ALAS1, B2M, HPRT1, POLR2A, PUM1, RPLP0, TBP, TUBA1B, and/or YWHAZ, e.g., at least one, or at least two, or at least three, or, preferably, all of these reference genes.

At step S106, a regression function for assigning a prediction of the therapy response or a personalization of the therapy is determined based on the first gene expression profiles for the two or more immune defense response genes, AIM2, APOBEC3A, CIAO1, DDX58, DHX9, IFI16, IFIH1, IFIT1, IFIT3, LRRFIP1, MYD88, OAS1, TLR8, and/or ZBP1, the second gene expression profiles for the two or more T-Cell receptor signaling genes, CD2, CD247, CD28, CD3E, CD3G, CD4, CSK, EZR, FYN, LAT, LCK, PAG1, PDE4D, PRKACA, PRKACB, PTPRC, and/or ZAP70, and the third gene expression profiles for the two or more PDE4D7 correlated genes, ABCC5, CUX2, KIAA1549, PDE4D, RAP1GAP2, SLC39A11, TDRD1, and/or VWA2, obtained for at least some of the biological samples obtained for the first set of patients and respective results obtained from the monitoring. In one particular realization, the regression function is determined as specified in Eq. (5) above.

At step S108, a biological sample is obtained from a patient (subject or individual). The patient can be a new patient or one of the first set.

At step S110, a first gene expression profile for each of one or more, for example, 1, 2, 3, 4, 5, 6, 7, 8, 9, 10, 11, 12, 13 or all, immune defense response genes selected from the group consisting of: AIM2, APOBEC3A, CIAO1, DDX58, DHX9, IFI16, IFIH1, IFIT1, IFIT3, LRRFIP1, MYD88, OAS1, TLR8, and ZBP1, a second gene expression profile for each of one or more, for example, 1, 2, 3, 4, 5, 6, 7, 8, 9, 10, 11, 12, 13, 14, 15, 16 or all, T-Cell receptor signaling genes selected from the group consisting of: CD2, CD247, CD28, CD3E, CD3G, CD4, CSK, EZR, FYN, LAT, LCK, PAG1, PDE4D, PRKACA, PRKACB, PTPRC, and ZAP70, and a third gene expression profile is obtained for each of the two or more, for example, 2, 3, 4, 5, 6, 7 or all, PDE4D7 correlated genes, e.g., by performing PCR on the biological sample. In one realization, the gene expression level for each of the two or more genes of the first gene expression profiles, the second gene expression profiles, and the third gene expression profiles is normalized with respect to one or more reference genes selected from the group consisting of ACTB, ALAS1, B2M, HPRT1, POLR2A, PUM1, RPLP0, TBP, TUBA1B, and/or YWHAZ, e.g., at least one, or at least two, or at least three, or, preferably, all of these reference genes. This is substantially the same as in step S104.

At step S112, a prediction of the therapy response or a personalization of the therapy based on the first, second, and third gene expression profiles is determined for the patient using the regression function. This will be described in more detail later in the description.

At S114, a therapy recommendation may be provided, e.g., to the patient or his or her guardian, to a doctor, or to another healthcare worker, based on the prediction or the personalization. To this end, the prediction or personalization may be categorized into one of a predefined set of risk groups, based on the value of the prediction or personalization. In one particular realization, the therapy may be radiotherapy and the prediction of the therapy response may be negative or positive for the effectiveness of the therapy. If the prediction is negative, the recommended therapy may comprise one or more of: (i) radiotherapy provided earlier than is the standard; (ii) radiotherapy with an increased radiation dose; (iii) an adjuvant therapy, such as androgen deprivation therapy; and (iv) an alternative therapy that is not a radiation therapy.

The method ends at S116.

In one embodiment, the gene expression profiles at steps S104 and S110 are determined by detecting mRNA expression using two or more primers and/or probes and/or two or more sets thereof.

In one embodiment, steps S104 and S110 further comprise obtaining clinical parameters from the first set of patients and the patient, respectively. The clinical parameters may comprise one or more of: (i) a prostate-specific antigen (PSA) level; (ii) a pathologic Gleason score (pGS); (iii) a clinical tumour stage; (iv) a pathological Gleason grade group (pGGG); (v) a pathological stage; (vi) one or more pathological variables, for example, a status of surgical margins and/or a lymph node invasion and/or an extra-prostatic growth and/or a seminal vesicle invasion; (vii) CAPRA; (viii) CAPRA-S; (ix) EAU-BCR risk groups, and; (x) another clinical risk score. The regression function for assigning the prediction of the therapy response or the personalization of the therapy that is determined in step S106 is then further based on the one or more clinical parameters obtained from at least some of the first set of patients. In step S112, the prediction of the therapy response or the personalization of the therapy is then further based on the one or more clinical parameters, e.g., the EAU-BCR risk groups, obtained from the patient and is determined for the patient using the regression function. In one particular realization, the regression function is determined as specified in Eq. (6) above.

Based on the significant correlation with outcome after SRT and/or SADT and/or CTX, we expect that the identified molecules will provide predictive value with regard to the effectiveness of radical RT and/or post-surgical SRT and/or SADT and/or CTX.

### Results

### TPM Gene Expression Values

For each gene a TPM (transcript per million) expression value was calculated based on the following steps:
1. Divide the read counts derived from the RNAseq fastq raw data after mapping and alignment to the human genome by the length of each gene in kilobases. This results in reads per kilobase (RPK).
2. Sum up all RPK values in a samples and divide this number by 1,000,000. This results in a ‘per million’ scaling factor.
3. Divide the RPK values by the “per million” scaling factor. This results in a TPM expression value for each gene.
   The TPM value per gene was used to calculate the reference normalized gene expression of each gene.

### Normalized Gene Expression Values

For the Cox regression modeling all gene TPM (transcript per million) based expression values from the RNAseq data were log 2 normalized by the following transformation:

TPM_log 2=log 2(TPM+1) (7)

In a second step of normalization of the TPM_log 2 expression values were normalized against the mean average of four reference genes (mean(refgenes)) as follows:

TPM_log 2_norm=log 2(TPM+1)−log 2(mean(ref_genes)) (8)

The following reference genes were considered (TABLE 1):

TABLE 1
Reference genes.

| Gene Symbol | Ensembl_ID |
|---|---|
| ALAS1 | ENSG00000023330 |
| ACTB | ENSG00000075624 |
| RPLP0 | ENSG00000089157 |
| TBP | ENSG00000112592 |
| TUBA1B | ENSG00000123416 |
| PUM1 | ENSG00000134644 |
| YWHAZ | ENSG00000164924 |
| HPRT1 | ENSG00000165704 |
| B2M | ENSG00000166710 |
| POLR2A | ENSG00000181222 |

For these reference genes, we selected the following four B2M, HPRT1, POLR2A, and PUM1 in order to calculate the:

log 2(mean(ref_genes))=AVERAGE((log 2(B2M+1), log 2(HPRT1+1), log 2(POLR2A+1), log 2(PUM1+1)) (9)

where the input data for the reference genes is their RNAseq measured gene expression in TPM (transcript per million), and AVERAGE is the mathematical mean.

In a final step, the reference gene normalized TPM_log 2_norm expression values for each gene were transformed into z-scores by calculating:

TPM_log 2_norm_mean_stdev=((TPM_log 2_norm)−(mean_samples))/(stdev_samples) (10)

where TPM_log 2_norm is the log 2, reference gene normalized TPM value per gene; mean_samples is the mathematical mean of TPM_log 2_norm vales across all samples and stdev_samples is the standard deviation of the TPM_lo2_norm values across all samples.

This process distributes the TPM_log 2_norm vales around the mean 0 with a standard deviation of 1.

For the multivariate analysis of the genes of interest we used the reference gene normalized log 2(TPM) z-score value of each gene as input.

### Cox Regression Analysis

We then set out to test whether the combination of the 14 immune defense response genes, the combination of the 17 T-Cell receptor signalling genes, the combination of the eight PDE4D7 correlated genes, and a combination thereof will exhibit more prognostic value. With Cox regression we modelled the expression levels of the 14 immune defense response genes, of the 17 T-Cell receptor signalling genes, and of the eight PDE4D7 correlated genes, respectively, to prostate cancer specific death after post-surgical salvage RT in a cohort of 571 prostate cancer patients.

The Cox regression functions were derived as follows:

(w₁·AIM2)+(w₂·APOBEC3A)+(w₃·CIAO1)+(w₄·DDX58)+(w₅·DHX9)+(w₆·IFI16)+(w₇·IFIH1)+(w₈·IFIT1)+(w₉·IFIT3)+(w₁₀·LRRFIP1)+(w₁₁·MYD88)+(w₁₂·OAS1)+(w₁₃·TLR8)+(w₁₄·ZBP1) IDR_model:

(w₁₅·C2)+(w₁₆·CD247)+(w₁₇·CD28)+(w₁₈·CD3E)+(w₁₉·CD3G)+(w₂₀·CD4)+(w₂₁·CSK)+(w₂₂·EZR)+(w₂₃·FYN)+(w₂₄·LAT)+(w₂₅·LCK)+(w₂₆·PAG1)+(w₂₇·PDE4D)+(w₂₈·PRKACA)+(w₂₉·PRKACB)+(w₃₀·PTPRC)+(w₃₁·ZAP70) TCR_SIGNALING_model:

(w₃₂·ABCC5)+(w₃₃·CUX2)+(w₃₄·KIAA1549)+(w₃₅·PDE4D)+(w₃₆·RAP1GAP2)+(w₃₇·SLC39A11)+(w₃₈·TDRD1)+(w₃₉·VWA2) PDE4D7_CORR_model:

The details for the weights w₁ to w₃₉ are shown in the following TABLE 2.

TABLE 2
Variables and weights for the three individual Cox regression models,
i.e., the immune defense response model (IDR_model), the T-Cell
receptor signaling model (TCR SIGNALING model), and the PDE4D7
correlation model (PDE4D7_CORR_model); NA - not available.

| | Weights | | | |
|---|---|---|---|---|
| | | | TCR_ | |
| Variable | | | SIGNALING_ | PDE4D7_ |
| Model | | IDR_model | model | CORR_model |
| AIM2 | w₁ | −0.313 | NA | NA |
| APOBEC3A | w₂ | −0.1417 | NA | NA |
| CIAO1 | w₃ | 0.1008 | NA | NA |
| DDX58 | w₄ | −0.07478 | NA | NA |
| DHX9 | w₅ | 0.277 | NA | NA |
| IFI16 | w₆ | −0.07944 | NA | NA |
| IFIH1 | w₇ | 0.3036 | NA | NA |
| IFIT1 | w₈ | −0.09188 | NA | NA |
| IFIT3 | w₉ | 0.1661 | NA | NA |
| LRRFIP1 | w₁₀ | −0.7105 | NA | NA |
| MYD88 | w₁₁ | 0.1615 | NA | NA |
| OAS1 | w₁₂ | −0.07468 | NA | NA |
| TLR8 | w₁₃ | −0.06677 | NA | NA |
| ZBP1 | w₁₄ | −0.155 | NA | NA |
| CD2 | w₁₅ | NA | 0.4137 | NA |
| CD247 | w₁₆ | NA | −0.1323 | NA |
| CD28 | w₁₇ | NA | 0.3695 | NA |
| CD3E | w₁₈ | NA | −0.267 | NA |
| CD3G | w₁₉ | NA | −0.1984 | NA |
| CD4 | w₂₀ | NA | 0.3018 | NA |
| CSK | w₂₁ | NA | 0.6169 | NA |
| EZR | w₂₂ | NA | −0.2789 | NA |
| FYN | w₂₃ | NA | −0.1842 | NA |
| LAT | w₂₄ | NA | 0.4672 | NA |
| LCK | w₂₅ | NA | −0.07028 | NA |
| PAG1 | w₂₆ | NA | 0.3278 | NA |
| PDE4D | w₂₇ | NA | −0.8253 | NA |
| PRKACA | w₂₈ | NA | 0.6212 | NA |
| PRKACB | w₂₉ | NA | −0.4462 | NA |
| PTPRC | w₃₀ | NA | −0.4622 | NA |
| ZAP70 | w₃₁ | NA | −0.3702 | NA |
| ABCC5 | w₃₂ | NA | NA | 0.368 |
| CUX2 | w₃₃ | NA | NA | −0.7675 |
| KIAA1549 | w₃₄ | NA | NA | 0.4108 |
| PDE4D | w₃₅ | NA | NA | 0.4007 |
| RAP1GAP2 | w₃₆ | NA | NA | −0.679 |
| SLC39A11 | w₃₇ | NA | NA | 0.5433 |
| TDRD1 | w₃₈ | NA | NA | 0.6366 |
| VWA2 | w₃₉ | NA | NA | −0.4749 |

Based on the three individual Cox regression models (IDR_model, TCR_SIGNALING_model, PDE4D7_CORR_model) we then again used Cox regression to model the combination thereof to prostate cancer specific death after post-surgical salvage RT either with (PCAI&Clinical_model) or without (PCAI_model) the presence of the variable EAU_BCR in the cohort of 571 prostate cancer patients. We tested the two models in Kaplan-Meier survival analysis.

The Cox regression functions were derived as follows:

(w₄₀·IDR_model)+(w₄₁·TCR_SIGNALING_model)+(w₄₂·PDE4D7_CORR_model) PCAI_model:

(w₄₃·PCAI_model)+(w₄₄·EAU_BCR) PCAI&Clinical_model:

The details for the weights w₄₀ to w₄₄ are shown in the following TABLE 3.

TABLE 3
Variables and weights for two combination Cox regression models,
i.e., prostate cancer AI model (PCAI_model) and the PCAI & clinical
model (PCAI&Clinical_model); NA - not available.

| Variable | Weights | | |
|---|---|---|---|
| Model | | PCAI_model | PCAI&Clinical |
| IDR_model | w₄₀ | 0.674 | NA |
| TCR_SIGNALING_ | w₄₁ | 0.5474 | NA |
| model | | | |
| PDE47_CORR_model | w₄₂ | 0.6372 | NA |
| PCAI_model | w₄₃ | NA | 0.8887 |
| EAU_BCR | w₄₄ | NA | 1.6085 |

### Kaplan-Meier Survival Analysis

For Kaplan-Meier survival curve analysis, the Cox functions of the risk models (PCAI_model and PCAI&Clinical_model) were categorized into three sub-cohorts based on a cut-off. The threshold for group separation into low, intermediate and high risk was based on the risk to experience the clinical endpoint as calculated by the Cox regression model. The Cox regression function calculated prognostic index (PI) was transformed to a 0-1 distribution by

PI(0-1)=1/(1+EXP(−PI)) (11)

where PI(0-1) is the prognostic index (PI) as calculated by the Cox regression function and EXP is the natural logarithmic function e.

For the PCAI_model the risk classes were defined as follows:

Low risk=PI(0-1)<0.6

Intermediate risk=PI(0-1)>=0.6 to <0.8

High risk=PI(0-1)>=0.1 to 1.0

For the PCAI&Clinical_model the risk categories were defined as follows:

Low risk=PI(0-1)<0.7

Intermediate risk=PI(0-1)>=0.7 to <0.9

High risk=PI(0-1)>=0.9 to 1.0

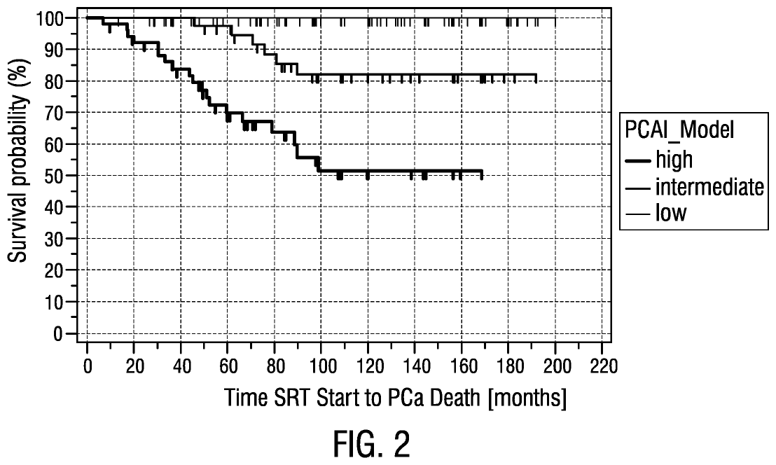

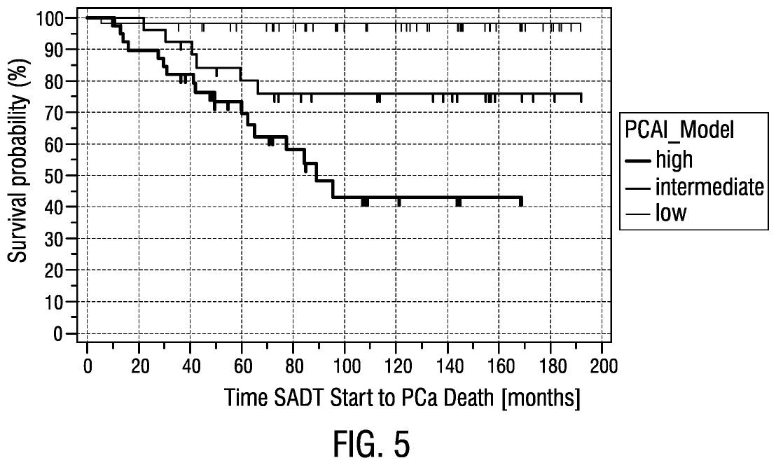

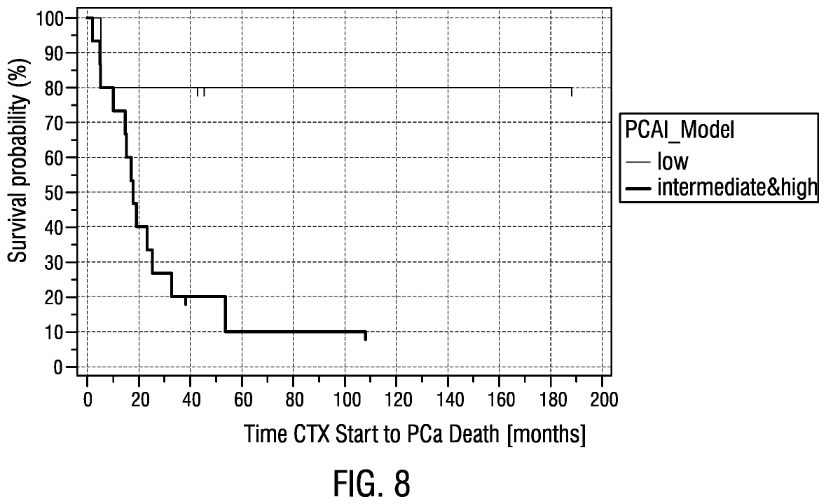

The patient classes represent an increasing risk to experience the tested clinical endpoints of prostate cancer specific death (PCa Death) and overall death after the start of salvage radiation therapy (SRT) due to post-surgical disease recurrence (FIGS. 2 to 4 and 9 to 11), prostate cancer specific death (PCa Death) and overall death after the start of salvage androgen deprivation therapy (SADT) due to post-surgical disease recurrence (FIGS. 5 to 7 and 12 to 14), and prostate cancer specific death (PCa death) after the start of cytotoxic chemotherapy (CTX) (FIG. 8) for the two created risk models (PCAI_model; PCAI&Clinical_model).

FIG. 2 shows a Kaplan-Meier curve of the PCAI_model in a 186 patient cohort (training set used to develop the PCAI_model) with all patients undergoing SRT (salvage radiation treatment) after post-surgical BCR (biochemical recurrence). The clinical endpoint that was tested was the prostate cancer specific death (PCa Death) after the start of salvage radiation therapy (SRT) due to post-surgical disease recurrence (log rank p<0.0001; HR low risk vs. intermediate risk=NA; 95% CI=NA; HR low risk vs. high risk=NA; 95% CI=NA; HR intermediate risk vs. high risk=3.6; 95% CI=1.1-11.6). The following supplementary lists indicate the number of patients at risk for the PCAI_model classes analyzed, i.e., the patients at risk at any time interval +20 months after SRT are shown: Low risk: 94, 93, 89, 82, 71, 53, 49, 36, 19, 9, 1, 0; Intermediate risk: 40, 40, 38, 35, 29, 21, 18, 13, 8, 2, 0, 0; High risk: 52, 46, 38, 27, 19, 12, 8, 6, 1, 0, 0, 0.

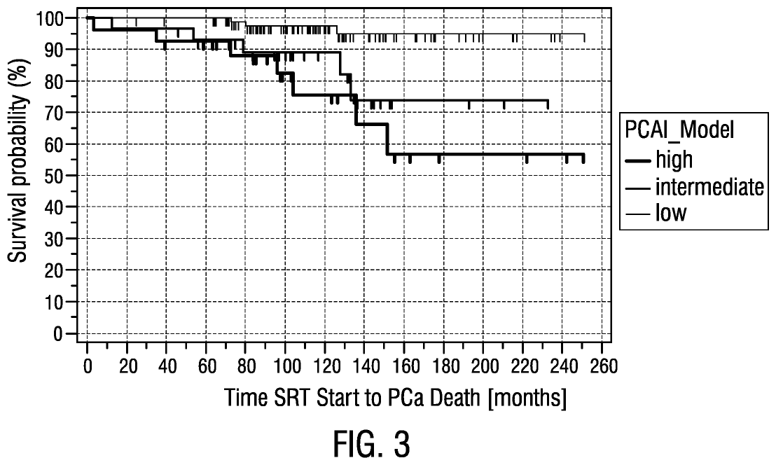

FIG. 3 shows a Kaplan-Meier curve of the PCAI_model in a 151 patient cohort (testing set used to validate the PCAI_model as developed on the 186 patient training set) with all patients undergoing SRT (salvage radiation treatment) after post-surgical BCR (biochemical recurrence). The clinical endpoint that was tested was the prostate cancer specific death (PCa Death) after the start of salvage radiation therapy (SRT) due to post-surgical disease recurrence (log rank p=0.001; HR low risk vs. intermediate risk=5.5; 95% CI=1.5-20.4; HR low risk vs. high risk=8.8; 95% CI=2.2-35.1; HR intermediate risk vs. high risk=1.6; 95% CI=0.3-8.6). The following supplementary lists indicate the number of patients at risk for the PCAI_model classes analyzed, i.e., the patients at risk at any time interval +20 months after SRT are shown: Low risk: 95, 95, 93, 93, 79, 59, 42, 28, 18, 12, 7, 4, 1, 0; Intermediate risk: 29, 28, 28, 25, 22, 18, 13, 8, 3, 3, 2, 1, 0, 0; High risk: 27, 26, 24, 23, 19, 13, 11, 7, 5, 3, 3, 3, 2, 0.

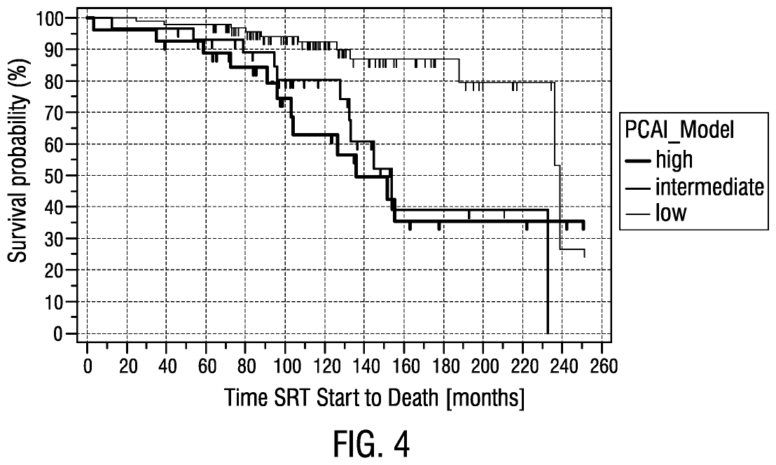

FIG. 4 shows a Kaplan-Meier curve of the PCAI_model in a 151 patient cohort (testing set used to validate the PCAI_model as developed on the 186 patient training set) with all patients undergoing SRT (salvage radiation treatment) after post-surgical BCR (biochemical recurrence). The clinical endpoint that was tested was the overall death after the start of salvage radiation therapy (SRT) due to post-surgical disease recurrence (log rank p=0.0009; HR low risk vs. intermediate risk=3.6; 95% CI=1.4-8.8; HR low risk vs. high risk=3.6; 95% CI=1.5-8.7; HR intermediate risk vs. high risk=1.0; 95% CI=0.3-3.1). The following supplementary lists indicate the number of patients at risk for the PCAI_model classes analyzed, i.e., the patients at risk at any time interval +20 months after SRT are shown: Low risk: 95, 95, 93, 93, 79, 59, 42, 28, 18, 12, 7, 4, 1, 0; Intermediate risk: 29, 28, 28, 25, 22, 18, 13, 8, 3, 3, 2, 1, 0, 0; High risk: 27, 26, 24, 23, 19, 13, 11, 7, 5, 3, 3, 3, 2, 0.

FIG. 5 shows a Kaplan-Meier curve of the PCAI_model in a 186 patient cohort (training set used to develop the PCAI_model) with all patients undergoing SADT (salvage androgen deprivation therapy) after post-surgical BCR (biochemical recurrence). The clinical endpoint that was tested was the prostate cancer specific death (PCa Death) after the start of salvage androgen deprivation therapy (SADT) due to post-surgical disease recurrence (log rank p<0.0001; HR low risk vs. intermediate risk=15.2; 95% CI=5.4-42.3; HR low risk vs. high risk=36.1; 95% CI=13.7-94.9; HR intermediate risk vs. high risk=2.4; 95% CI=0.7-7.7). The following supplementary lists indicate the number of patients at risk for the PCAI_model classes analyzed, i.e., the patients at risk at any time interval +20 months after SADT are shown: Low risk: 59, 57, 56, 52, 47, 34, 32, 25, 13, 6, 0; Intermediate risk: 26, 26, 23, 19, 16, 14, 12, 10, 4, 2, 0; High risk: 59, 57, 56, 52, 47, 34, 32, 25, 13, 6, 0.

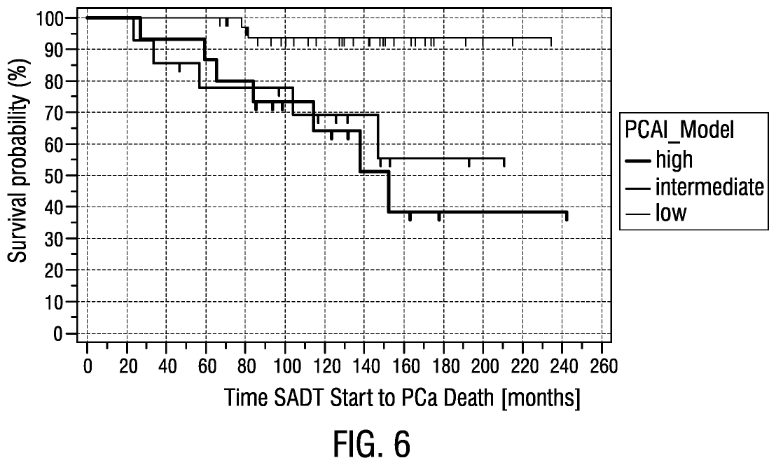

FIG. 6 shows a Kaplan-Meier curve of the PCAI_model in a 151 patient cohort (testing set used to validate the PCAI_model as developed on the 186 patient training set) with all patients undergoing SADT (salvage androgen deprivation therapy) after post-surgical BCR (biochemical recurrence). The clinical endpoint that was tested was the prostate cancer specific death (PCa Death) after the start of salvage androgen deprivation therapy (SADT) due to post-surgical disease recurrence (log rank p=0.003; HR low risk vs. intermediate risk=7.6; 95% CI=1.9-30.0; HR low risk vs. high risk=9.5; 95% CI=2.6-35.4; HR intermediate risk vs. high risk=1.2; 95% CI=0.2-6.4). The following supplementary lists indicate the number of patients at risk for the PCAI_model classes analyzed, i.e., the patients at risk at any time interval +20 months after SADT are shown: Low risk: 37, 37, 37, 37, 32, 24, 20, 16, 10, 5, 3, 1, 0, 0; Intermediate risk: 14, 14, 12, 10, 10, 9, 7, 5, 2, 2, 1, 0, 0, 0; High risk: 15, 15, 14, 13, 12, 8, 7, 4, 3, 1, 1, 1, 1, 0.

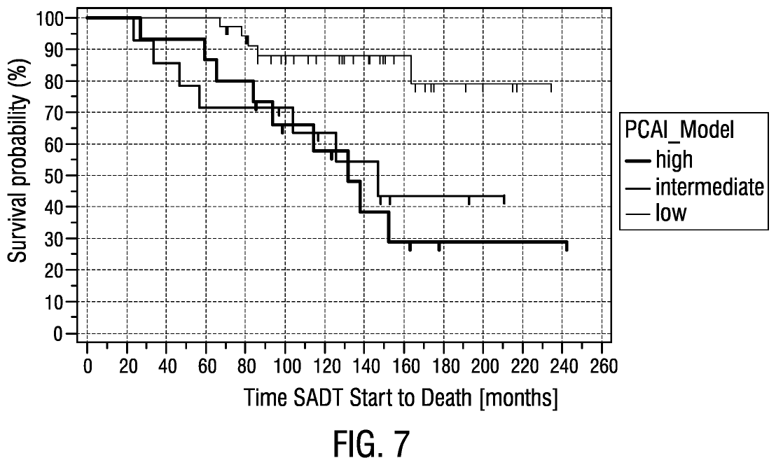

FIG. 7 shows a Kaplan-Meier curve of the PCAI_model in a 151 patient cohort (testing set used to validate the PCAI_model as developed on the 186 patient training set) with all patients undergoing SADT (salvage androgen deprivation therapy) after post-surgical BCR (biochemical recurrence). The clinical endpoint that was tested was the overall death after the start of salvage androgen deprivation therapy (SADT) due to post-surgical disease recurrence (log rank p=0.003; HR low risk vs. intermediate risk=7.6; 95% CI=1.9-30.0; HR low risk vs. high risk=9.5; 95% CI=2.6-35.4; HR intermediate risk vs. high risk=1.2; 95% CI=0.2-6.4). The following supplementary lists indicate the number of patients at risk for the PCAI_model classes analyzed, i.e., the patients at risk at any time interval +20 months after SADT are shown: Low risk: 37, 37, 37, 37, 32, 24, 20, 16, 10, 5, 3, 1, 0, 0; Intermediate risk: 14, 14, 12, 10, 10, 9, 7, 5, 2, 2, 1, 0, 0, 0; High risk: 15, 15, 14, 13, 12, 8, 7, 4, 3, 1, 1, 1, 1, 0.

FIG. 8 shows a Kaplan-Meier curve of the PCAI_model in a 186 patient cohort (training set used to develop the PCAI_model) with all patients undergoing CTX (cytotoxic chemotherapy) after post-surgical BCR (biochemical recurrence). The clinical endpoint that was tested was the prostate cancer specific death (PCa Death) after the start of cytotoxic chemotherapy (CTX) due to post-surgical disease recurrence (log rank p=0.03; HR low risk vs. intermediate&high risk=3.4; 95% CI=1.1-10.6). The following supplementary lists indicate the number of patients at risk for the PCAI_model classes analyzed, i.e., the patients at risk at any time interval +20 months after CTX are shown: Low risk: 5, 4, 4, 2, 2, 2, 2, 2, 2, 2, 0; Intermediate&high risk: 15, 6, 2, 1, 1, 1, 0, 0, 0, 0, 0.

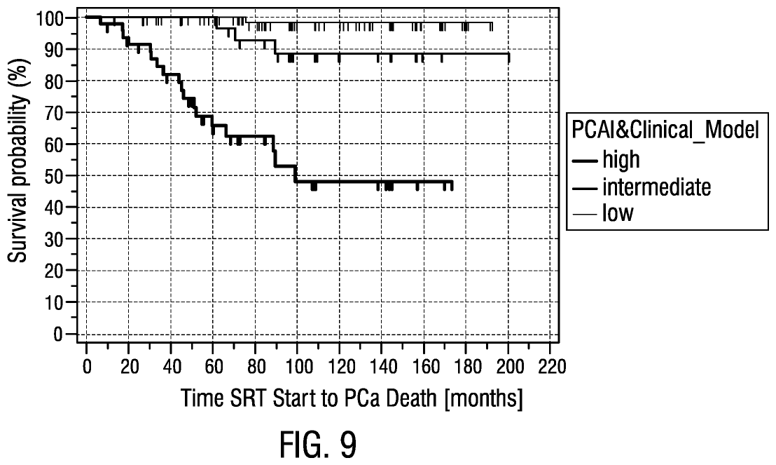

FIG. 9 shows a Kaplan-Meier curve of the PCAI&Clinical_model in a 186 patient cohort (training set used to develop the PCAI&Clinical_model) with all patients undergoing SRT (salvage radiation treatment) after post-surgical BCR (biochemical recurrence). The clinical endpoint that was tested was the prostate cancer specific death (PCa Death) after the start of salvage radiation therapy (SRT) due to post-surgical disease recurrence (log rank p<0.0001; HR low risk vs. intermediate risk=7.4; 95% CI=2.6-20.9; HR low risk vs. high risk=46.9; 95% CI=16.2-135.0; HR intermediate risk vs. high risk=6.3; 95% CI=1.8-22.3). The following supplementary lists indicate the number of patients at risk for the PCAI&Clinical_model classes analyzed, i.e., the patients at risk at any time interval +20 months after SRT are shown: Low risk: 79, 79, 75, 69, 60, 42, 37, 28, 15, 5, 0, 0; Intermediate risk: 33, 32, 31, 30, 23, 15, 12, 9, 2, 1, 1, 0; High risk: 47, 41, 32, 21, 16, 10, 7, 6, 2, 0, 0, 0.

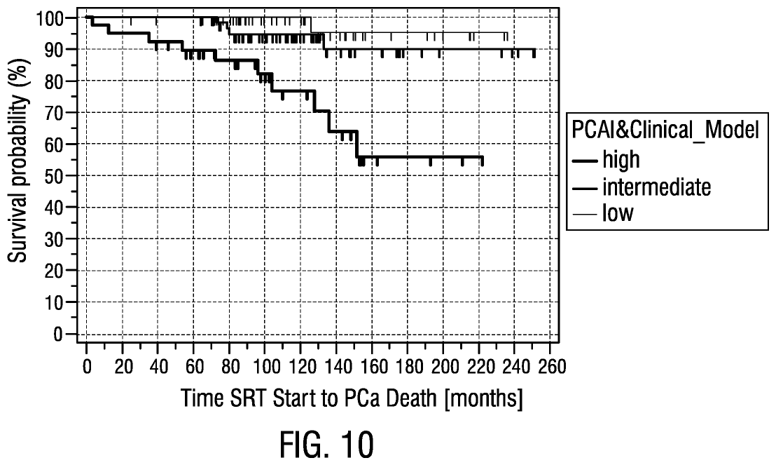

FIG. 10 shows a Kaplan-Meier curve of the PCAI&Clinical_model in a 151 patient cohort (testing set used to validate the PCAI&Clinical_model as developed on the 186 patient training set) with all patients undergoing SRT (salvage radiation treatment) after post-surgical BCR (biochemical recurrence). The clinical endpoint that was tested was the prostate cancer specific death (PCa Death) after the start of salvage radiation therapy (SRT) due to post-surgical disease recurrence (log rank p=0.0002; HR low risk vs. intermediate risk=3.2; 95% CI=1.0-10.3; HR low risk vs. high risk=15.4; 95% CI=3.9-60.6; HR intermediate risk vs. high risk=4.7; 95% CI=1.3-17.7). The following supplementary lists indicate the number of patients at risk for the PCAI&Clinical_model classes analyzed, i.e., the patients at risk at any time interval +20 months after SRT are shown: Low risk: 52, 52, 50, 50, 41, 29, 25, 16, 9, 8, 5, 2, 0, 0; Intermediate risk: 60, 60, 60, 60, 53, 42, 28, 17, 13, 7, 5, 5, 3, 0; High risk: 39, 37, 35, 31, 26, 19, 13, 10, 4, 3, 2, 1, 0, 0.

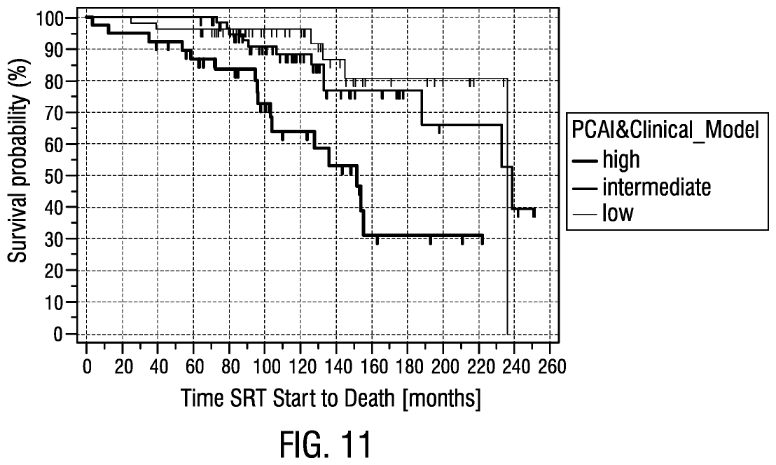

FIG. 11 shows a Kaplan-Meier curve of the PCAI&Clinical_model in a 151 patient cohort (testing set used to validate the PCAI&Clinical_model as developed on the 186 patient training set) with all patients undergoing SRT (salvage radiation treatment) after post-surgical BCR (biochemical recurrence). The clinical endpoint that was tested was the overall death after the start of salvage radiation therapy (SRT) due to post-surgical disease recurrence (log rank p=0.0003; HR low risk vs. intermediate risk=1.4; 95% CI=0.7-3.0; HR low risk vs. high risk=4.4; 95% CI=1.7-11.5; HR intermediate risk vs. high risk=3.1; 95% CI=1.3-7.7). The following supplementary lists indicate the number of patients at risk for the PCAI&Clinical_model classes analyzed, i.e., the patients at risk at any time interval +20 months after SRT are shown: Low risk: 52, 52, 50, 50, 41, 29, 25, 16, 9, 8, 5, 2, 0, 0; Intermediate risk: 60, 60, 60, 60, 53, 42, 28, 17, 13, 7, 5, 5, 3, 0; High risk: 39, 37, 35, 31, 26, 19, 13, 10, 4, 3, 2, 1, 0, 0.

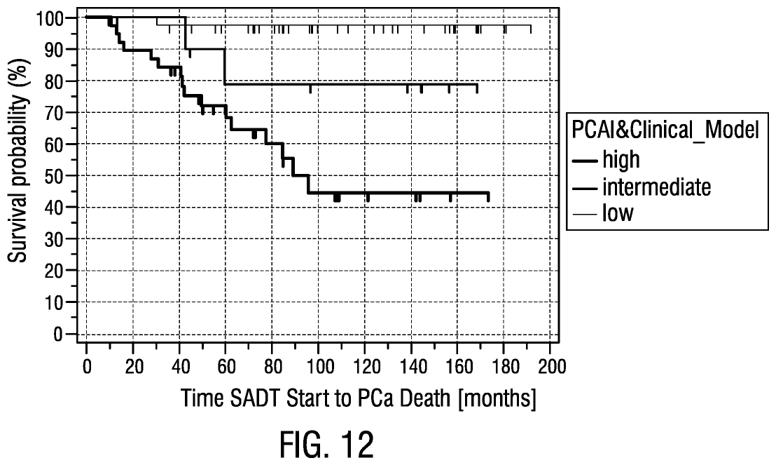

FIG. 12 shows a Kaplan-Meier curve of the PCAI&Clinical_model in a 186 patient cohort (training set used to develop the PCAI_model) with all patients undergoing SADT (salvage androgen deprivation therapy) after post-surgical BCR (biochemical recurrence). The clinical endpoint that was tested was the prostate cancer specific death (PCa Death) after the start of salvage androgen deprivation therapy (SADT) due to post-surgical disease recurrence (log rank p<0.0001; HR low risk vs. intermediate risk=8.2; 95% CI=2.0-33.3; HR low risk vs. high risk=24.6; 95% CI=9.2-65.9; HR intermediate risk vs. high risk=3.0; 95% CI=0.7-13.0). The following supplementary lists indicate the number of patients at risk for the PCAI&Clinical_model classes analyzed, i.e., the patients at risk at any time interval +20 months after SADT are shown: Low risk: 41, 41, 39, 36, 31, 20, 18, 14, 7, 3, 0; Intermediate risk: 11, 10, 10, 7, 7, 5, 5, 4, 1, 0, 0; High risk: 39, 34, 28, 20, 14, 8, 5, 4, 1, 0, 0.

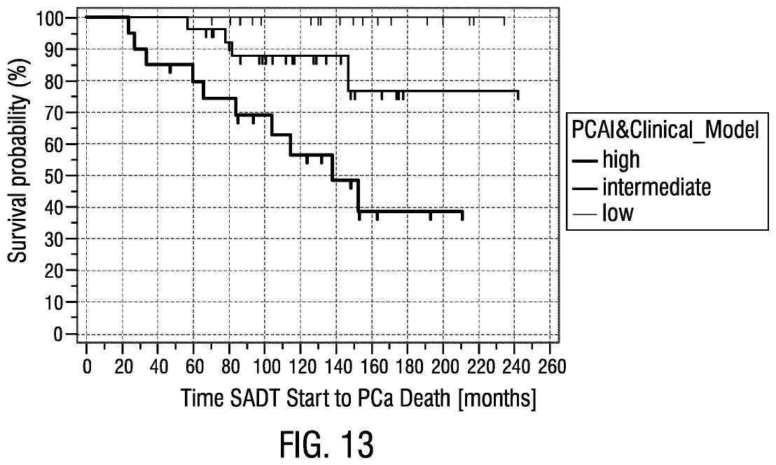

FIG. 13 shows a Kaplan-Meier curve of the PCAI&Clinical_model in a 151 patient cohort (testing set used to validate the PCAI_model as developed on the 186 patient training set) with all patients undergoing SADT (salvage androgen deprivation therapy) after post-surgical BCR (biochemical recurrence). The clinical endpoint that was tested was the prostate cancer specific death (PCa Death) after the start of salvage androgen deprivation therapy (SADT) due to post-surgical disease recurrence (log rank p=0.0005; HR low risk vs. intermediate risk=NA; 95% CI=NA; HR low risk vs. high risk=NA; 95% CI=NA; HR intermediate risk vs. high risk=3.6; 95% CI=1.0-13.4). The following supplementary lists indicate the number of patients at risk for the PCAI&Clinical_model classes analyzed, i.e., the patients at risk at any time interval +20 months after SADT are shown: Low risk: 19, 19, 19, 19, 18, 13, 13, 10, 7, 5, 3, 1, 0, 0; Intermediate risk: 27, 27, 27, 26, 22, 17, 12, 9, 5, 1, 1, 1, 1, 0; High risk: 20, 20, 17, 15, 14, 11, 9, 6, 3, 2, 1, 0, 0, 0.

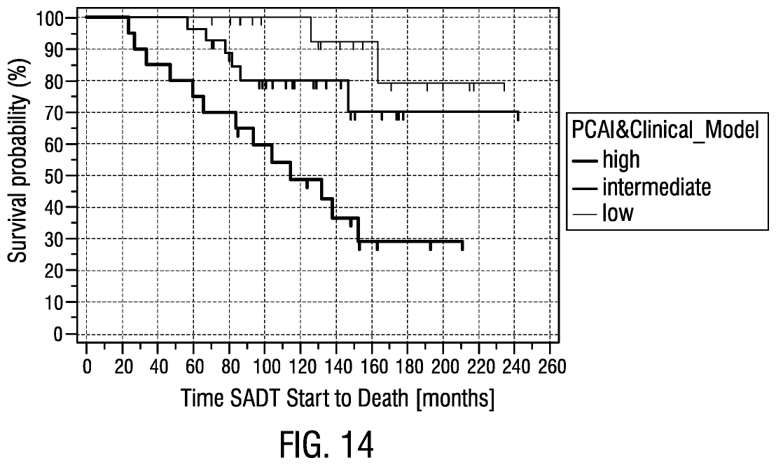

FIG. 14 shows a Kaplan-Meier curve of the PCAI&Clinical_model in a 151 patient cohort (testing set used to validate the PCAI_model as developed on the 186 patient training set) with all patients undergoing SADT (salvage androgen deprivation therapy) after post-surgical BCR (biochemical recurrence). The clinical endpoint that was tested was the overall death after the start of salvage androgen deprivation therapy (SADT) due to post-surgical disease recurrence (log rank p=0.0006; HR low risk vs. intermediate risk=2.76; 95% CI=1.0-7.1; HR low risk vs. high risk=8.5; 95% CI=2.8-25.9; HR intermediate risk vs. high risk=3.3 95% CI=1.1-9.6). The following supplementary lists indicate the number of patients at risk for the PCAI&Clinical_model classes analyzed, i.e., the patients at risk at any time interval +20 months after SADT are shown: Low risk: 19, 19, 19, 19, 18, 13, 13, 10, 7, 5, 3, 1, 0, 0; Intermediate risk: 27, 27, 27, 26, 22, 17, 12, 9, 5, 1, 1, 1, 1, 1, 0; High risk: 20, 20, 17, 15, 14, 11, 9, 6, 3, 2, 1, 0, 0, 0.

The Kaplan-Meier survival curve analysis as shown in FIGS. 2 to 14 demonstrates the presence of different patient risk groups. The risk group of a patient is determined by the probability to suffer from the respective clinical endpoint (death, prostate cancer specific death) as calculated by the risk model PCAI_model or PCAI&Clinical_model. Depending on the predicted risk of a patient (i.e., depending on in which risk group the patient may belong) to die from prostate cancer different types of interventions might be indicated. In the lower risk groups standard of care (SOC), which is SRT potentially combined with SADT (salvage androgen deprivation therapy), delivers acceptable long-term oncological control. For the patient group demonstrating a higher risk to eventually suffer from prostate cancer death dose escalation of the applied RT and/or combination with chemotherapy might provide improved longitudinal survival outcomes. Alternative options for treatment escalation are early combination of SRT (considering higher dose regimens), SADT, and chemotherapy or alternative therapies like immunotherapies (e.g., Sipuleucil-T) or other experimental therapies.

### Further Results

The inventors speculated that use of the full model model (i.e. using all 8 PDE4D7 correlated genes, all 14 immune defense response genes and all 17 T-Cell receptor signaling genes) would not be essential to obtain a significant predictive effect. Therefore, a random selection of one of the PDE4D7 correlated genes combined with one randomly selected immune defense response gene and one randomly selected T-Cell receptor signaling gene were selected from the full set to investigate whether a set of one gene selected from each group would suffice to make a prediction.

This section shows additional results for Cox regression models based on six different gene models comprising randomly selected combinations of three genes, wherein said three genes comprise per random combination of genes. In five of the six gene models (model 3.1, 3.2., 3.4. −3.6) the random combination of genes comprises one immune defense response gene, one TCR signaling gene and one PDE4D7 correlated gene. In one reference model (model 3.3) the random combination of genes comprises two TCR signaling gene and one PDE4D7 correlated gene. The details for the variables and weights are displayed in TABLE 4.

TABLE 4
Variables and weights for the six gene Cox regression models, i.e. the five gene models
comprising a randomly selected combination of three genes, having per random combination one immune
defense response gene, one TCR signaling gene and one PDE4D7 correlated gene (model 3.1, 3.2., 3.4. -
3.6) and one reference (model 3.3).

| | | PCAI-3 combinations regression models | | | | | |
|---|---|---|---|---|---|---|---|
| | Variable | 3.1 | 3.2 | 3.3 (reference) | 3.4 | 3.5 | 3.6 |
| Immune | AIM2 | — | — | — | — | — | — |
| defense | APOBEC3A | — | — | — | — | — | — |
| response | CIAO1 | — | — | — | — | — | — |
| genes | DDX58 | — | — | — | — | — | — |
| | DHX9 | — | — | — | — | — | — |
| | IFI16 | −0,3148 | — | — | — | — | — |
| | IFIT1 | — | — | — | — | — | −0,2293 |
| | IFIT3 | — | — | — | −0,1308 | — | — |
| | LRRFIP1 | — | −0,6686 | — | — | — | — |
| | MYD88 | — | — | — | — | — | — |
| | OAS1 | — | — | — | — | −0,07831 | — |
| | TLR8 | — | — | — | — | — | — |
| | ZBP1 | — | — | — | — | — | — |
| TCR | CD2 | — | — | — | — | — | — |
| signaling | CD247 | — | — | — | — | — | — |
| genes | CD28 | — | — | — | — | — | — |
| | CD3E | — | — | −0,06166 | — | — | — |
| | CD3G | — | — | — | — | — | — |
| | CD4 | — | — | — | — | — | — |
| | CSK | — | — | — | — | — | 0,1881 |
| | EZR | — | — | — | −0,1059 | — | — |
| | FYN | — | −0,1393 | — | — | — | — |
| | LAT | — | — | — | — | — | — |
| | LCK | — | — | — | — | — | — |
| | PAG1 | — | — | — | — | — | — |
| | PDE4D | — | — | −0,4176 | — | — | — |
| | PRKACA | 0,3535 | — | — | — | — | — |
| | PRKACB | — | — | — | — | — | — |
| | PTPRC | — | — | — | — | −0,2034 | — |
| | ZAP70 | — | — | — | — | — | — |
| PDE4D7 | ABCC5 | — | — | — | 0,3185 | — | — |
| correlated | CUX2 | — | 0,2883 | — | — | — | — |
| genes | KIAA1549 | — | — | — | — | — | — |
| | PDE4D | — | — | — | — | −0,3429 | — |
| | RAP1GAP2 | −0,4725 | — | — | — | — | — |
| | SLC39A11 | — | — | — | — | — | 0,3593 |
| | TDRD1 | — | — | — | — | — | — |
| | VWA2 | — | — | 0,1159 | — | — | — |

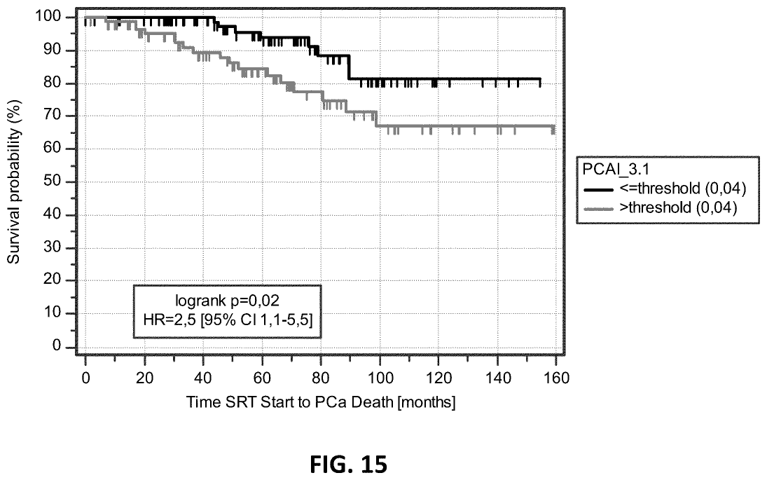

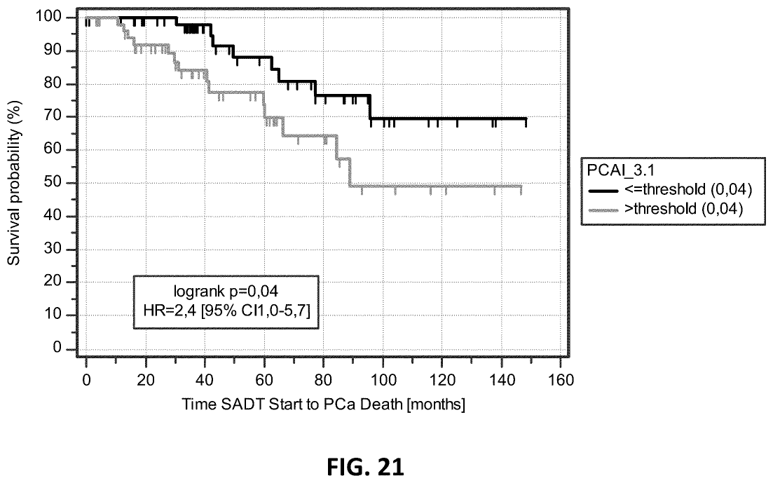

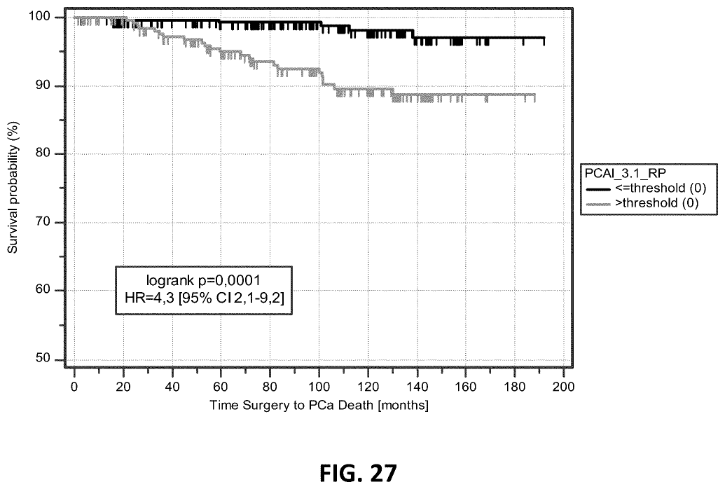

For Kaplan-Meier curve analysis the Cox regression function of the six risk models (PCAI-3.1-3.6) was categorized into two sub-cohorts (low risk vs. high risk) based on a cut-off (see description of figures below). The patient cohorts were tested for three different endpoints: prostate cancer specific death (PCa Death) after start of SRT (salvage radiation) in a 186 patients cohort (FIGS. 15-20), prostate cancer specific death (PCa Death) after start of SADT (salvage androgen deprivation) in a 106 patients cohort (FIGS. 21-26) or prostate cancer specific death (PCa Death) after surgery in a 571 patient cohort (FIGS. 27-32). The endpoint seeing to PCa Death after surgery comprises patients that have not been treated with SRT. In other words, testing by using the models of the present invention may be applied to predict an endpoint before starting a primary treatment in prostate cancer patients.

FIG. 15 shows a Kaplan-Meier curve for the PCAI-3.1 model. The clinical endpoint tested was PCa Death after start of SRT due to post-surgical disease recurrence. Subjects were stratified into two cohorts according to their risk to experience the clinical endpoint as predictend by PCAI-3.1 model (low vs. high risk). The value 0.04 was used as cut-off (log rank p=0.02; HR=2.5; CI=1.1-5.5). The following supplementary list indicate the number of subjects at risk for the analyzed PCAI-3.1 model classes, i.e. the subjects at risk at any time interval +20 months after surgery are shown: Low Risk (<=0.04): 97, 90, 73, 55, 31, 17, 6, 3, 0; High risk (>0.04): 88, 73, 58, 43, 29, 16, 9, 5, 0.

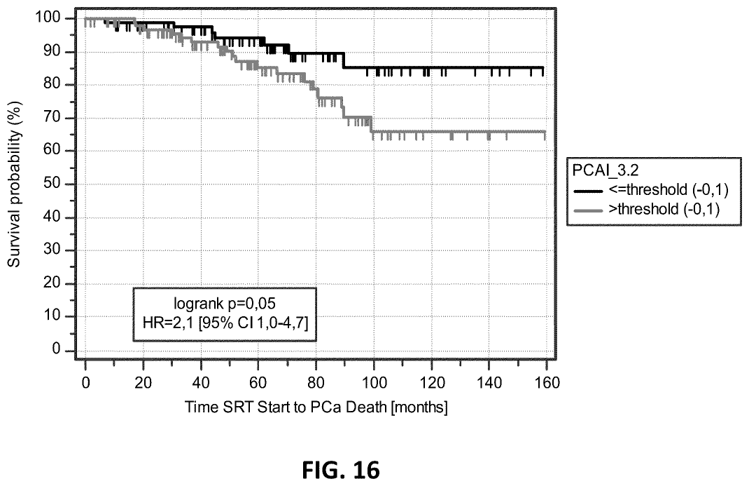

FIG. 16 shows a Kaplan-Meier curve for the PCAI-3.2 model. The clinical endpoint tested was PCa Death after start of SRT due to post-surgical disease recurrence. Subjects were stratified into two cohorts according to their risk to experience the clinical endpoint as predictend by PCAI-3.2 model (low vs. high risk). The value −0.1 was used as cut-off (log rank p=0.05; HR=2.1; CI=1.0-4.7). The following supplementary list indicate the number of subjects at risk for the analyzed PCAI-3.2 model classes, i.e. the subjects at risk at any time interval +20 months after surgery are shown: Low Risk (<−0.1): 83, 75, 64, 49, 27, 19, 8, 5, 0; High risk (>−0.1): 102, 88, 67, 49, 33, 14, 7, 3, 0.

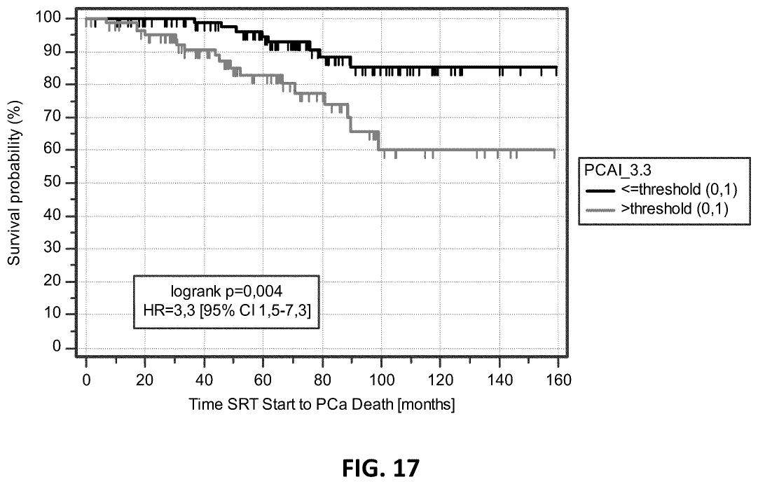

FIG. 17 comprises a reference figure showing a Kaplan-Meier curve for the PCAI-3.3 model. The clinical endpoint tested was PCa Death after start of SRT due to post-surgical disease recurrence. Subjects were stratified into two cohorts according to their risk to experience the clinical endpoint as predictend by reference PCAI-3.3 model (low vs. high risk). The value 0.1 was used as cut-off (log rank p=0.004; HR=3.3; CI=1.5-7.3). The following supplementary list indicate the number of subjects at risk for the analyzed reference PCAI-3.3 model classes, i.e. the subjects at risk at any time interval +20 months after surgery are shown: Low Risk (<=0.1): 100, 89, 76, 61, 37, 22, 9, 5, 0; High risk (>0.1): 85, 74, 55, 37, 23, 11, 6, 3, 0.

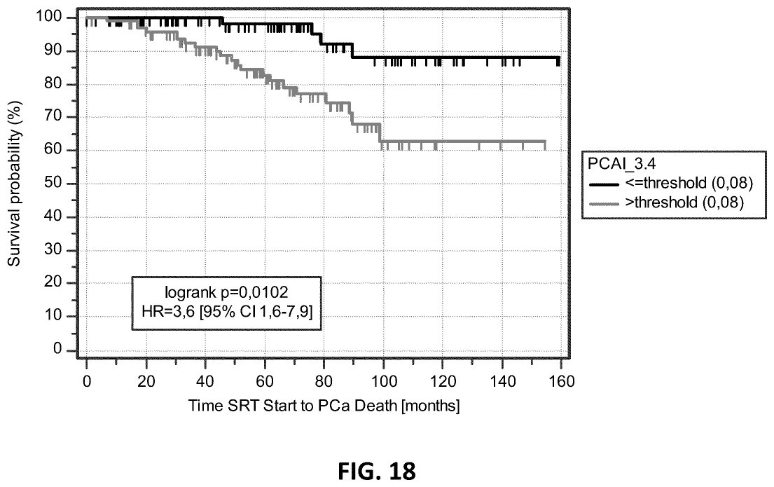

FIG. 18 shows a Kaplan-Meier curve for the PCAI-3.4 model. The clinical endpoint tested was PCa Death after start of SRT due to post-surgical disease recurrence. Subjects were stratified into two cohorts according to their risk to experience the clinical endpoint as predictend by PCAI-3.4 model (low vs. high risk). The value 0.08 was used as cut-off (log rank p=0.0102; HR=3.6; CI=1.6-7.9). The following supplementary list indicate the number of subjects at risk for the analyzed PCAI-3.4 model classes, i.e. the subjects at risk at any time interval +20 months after surgery are shown: Low Risk (<=0.08): 85, 72, 59, 48, 29, 22, 11, 6, 0; High risk (>0.08): 100, 91, 72, 50, 31, 11, 4, 2, 0

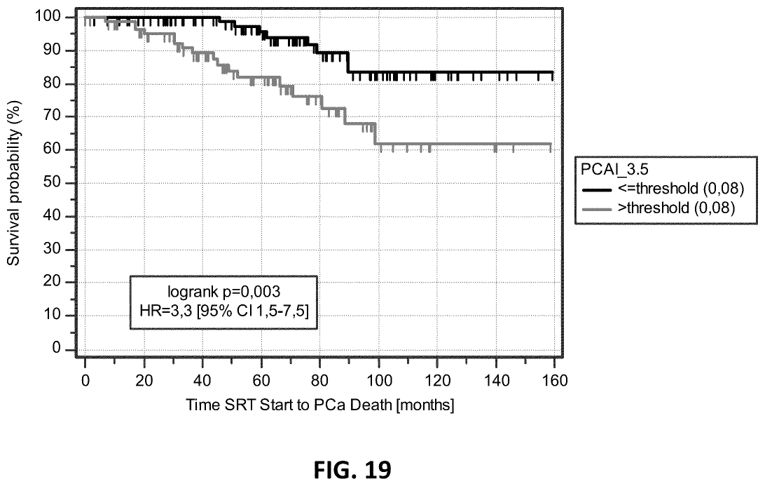

FIG. 19 shows a Kaplan-Meier curve for the PCAI-3.5 model. The clinical endpoint tested was PCa Death after start of SRT due to post-surgical disease recurrence. Subjects were stratified into two cohorts according to their risk to experience the clinical endpoint as predictend by PCAI-3.5 model (low vs. high risk). The value 0.08 was used as cut-off (log rank p=0.003; HR=3.3; CI=1.5-7.5). The following supplementary list indicate the number of subjects at risk for the analyzed PCAI-3.5 model classes, i.e. the subjects at risk at any time interval +20 months after surgery are shown: Low Risk (<=0.08): 99, 90, 75, 59, 38, 23, 11, 5, 0; High risk (>0.08): 86, 73, 56, 39, 22, 10, 4, 3, 0.

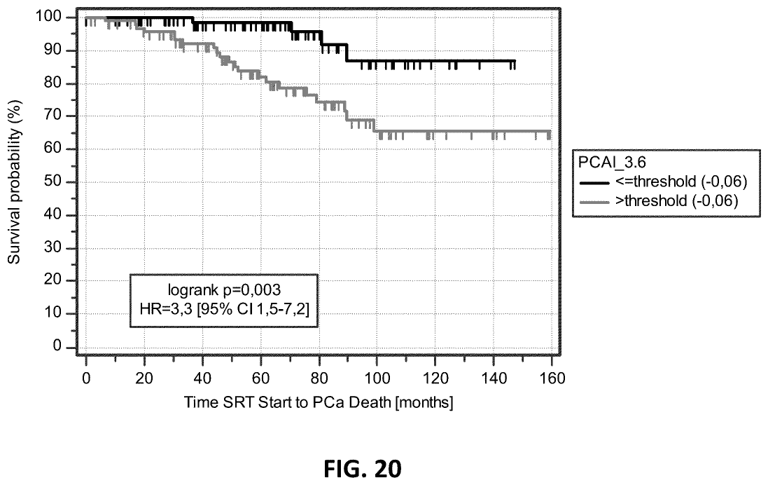

FIG. 20 shows a Kaplan-Meier curve for the PCAI-3.6 model. The clinical endpoint tested was PCa Death after start of SRT due to post-surgical disease recurrence. Subjects were stratified into two cohorts according to their risk to experience the clinical endpoint as predictend by PCAI-3.6 model (low vs. high risk). The value −0.06 was used as cut-off (log rank p=0.003; HR=3.3; CI=1.5-7.2). The following supplementary list indicate the number of subjects at risk for the analyzed PCAI-3.6 model classes, i.e. the subjects at risk at any time interval +20 months after surgery are shown: Low Risk (<=−0.06): 86, 76, 58, 49, 25, 14, 6, 2, 0; High risk (>−0.06): 99, 87, 73, 49, 35, 19, 9, 6, 0.

FIG. 21 shows a Kaplan-Meier curve for the PCAI-3.1 model. The clinical endpoint tested was PCa Death after start of SADT due to post-surgical disease recurrence. Subjects were stratified into two cohorts according to their risk to experience the clinical endpoint as predictend by PCAI-3.1 model (low vs. high risk). The value 0.04 was used as cut-off (log rank p=0.04; HR=2.4; CI=1.0-5.7). The following supplementary list indicate the number of subjects at risk for the analyzed PCAI-3.1 model classes, i.e. the subjects at risk at any time interval +20 months after surgery are shown: Low Risk (<=0.04): 54, 47, 31, 24, 17, 9, 4, 1, 0; High risk (>0.04): 52, 41, 26, 19, 11, 5, 3, 1, 0.

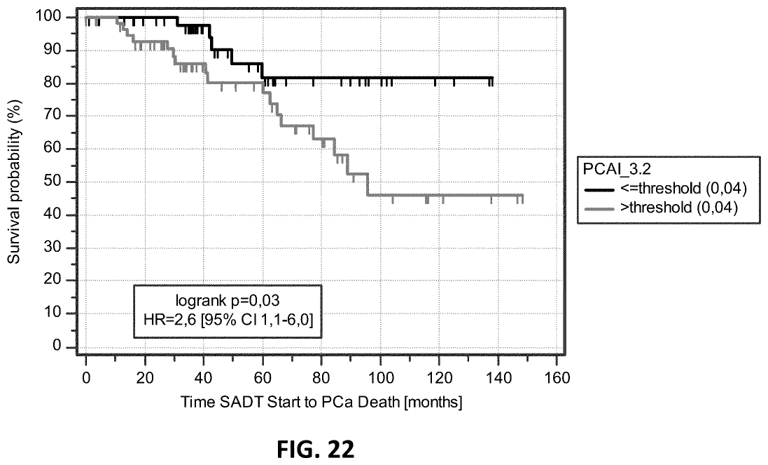

FIG. 22 shows a Kaplan-Meier curve for the PCAI-3.2 model. The clinical endpoint tested was PCa Death after start of SADT due to post-surgical disease recurrence. Subjects were stratified into two cohorts according to their risk to experience the clinical endpoint as predictend by PCAI-3.2 model (low vs. high risk). The value 0.04 was used as cut-off (log rank p=0.03; HR=2.6; CI=1.1-6.0). The following supplementary list indicate the number of subjects at risk for the analyzed PCAI-3.2 model classes, i.e. the subjects at risk at any time interval +20 months after surgery are shown: Low Risk (<=0.04): 48, 41, 27, 18, 12, 7, 3, 0, 0; High risk (>0.04): 58, 47, 30, 25, 16, 7, 4, 2, 0.

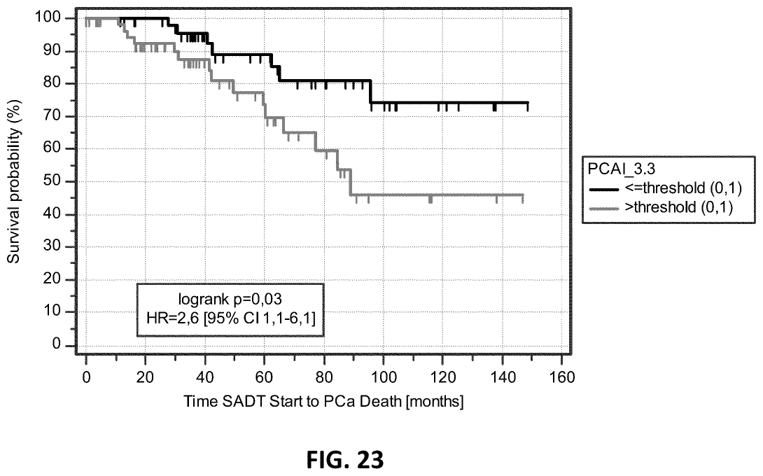

FIG. 23 comprises a reference figure showing a Kaplan-Meier curve for the PCAI-3.3 model. The clinical endpoint tested was PCa Death after start of SADT due to post-surgical disease recurrence. Subjects were stratified into two cohorts according to their risk to experience the clinical endpoint as predictend by reference PCAI-3.3 model (low vs. high risk). The value 0.1 was used as cut-off (log rank p=0.03; HR=2.6; CI=1.1-6.1). The following supplementary list indicate the number of subjects at risk for the analyzed reference PCAI-3.3 model classes, i.e. the subjects at risk at any time interval +20 months after surgery are shown: Low Risk (<=0.1): 50, 45, 30, 24, 17, 10, 5, 1, 0; High risk (>0.1): 56, 43, 27, 19, 11, 4, 2, 1, 0.

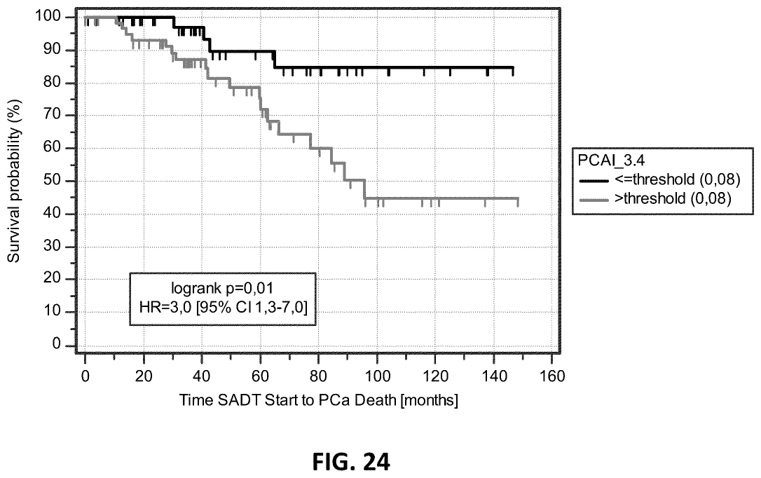

FIG. 24 shows a Kaplan-Meier curve for the PCAI-3.4 model. The clinical endpoint tested was PCa Death after start of SADT due to post-surgical disease recurrence. Subjects were stratified into two cohorts according to their risk to experience the clinical endpoint as predictend by PCAI-3.4 model (low vs. high risk). The value 0,08 was used as cut-off (log rank p=0.01; HR=3.0; CI=1.3-7.0). The following supplementary list indicate the number of subjects at risk for the analyzed PCAI-3.4 model classes, i.e. the subjects at risk at any time interval +20 months after surgery are shown: Low Risk (<=0.08): 46, 36, 26, 20, 14, 7, 4, 1, 0; High risk (>0.08): 60, 52, 31, 23, 14, 7, 3, 1, 0.

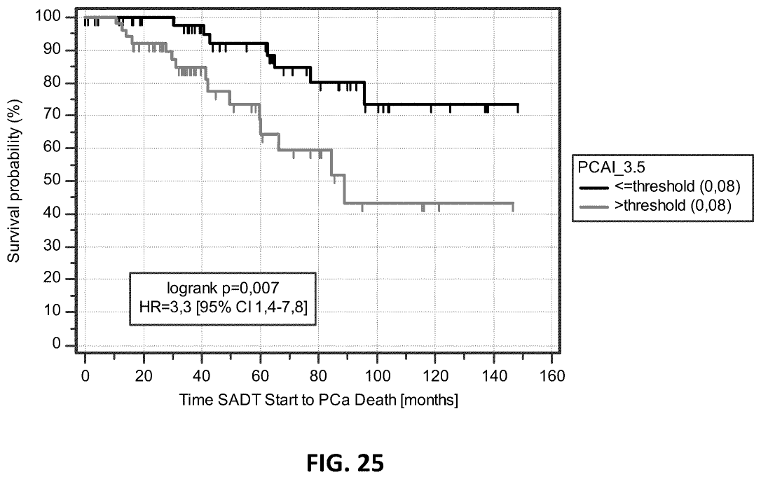

FIG. 25 shows a Kaplan-Meier curve for the PCAI-3.5 model. The clinical endpoint tested was PCa Death after start of SADT due to post-surgical disease recurrence. Subjects were stratified into two cohorts according to their risk to experience the clinical endpoint as predictend by PCAI-3.5 model (low vs. high risk). The value 0.08 was used as cut-off (log rank p=0.007; HR=3.3; CI=1.4-7.8). The following supplementary list indicate the number of subjects at risk for the analyzed PCAI-3.5 model classes, i.e. the subjects at risk at any time interval +20 months after surgery are shown: Low Risk (<=0.08): 54, 44, 34, 28, 18, 10, 5, 1, 0; High risk (>0.08): 52, 44, 23, 15, 10, 4, 2, 1, 0.

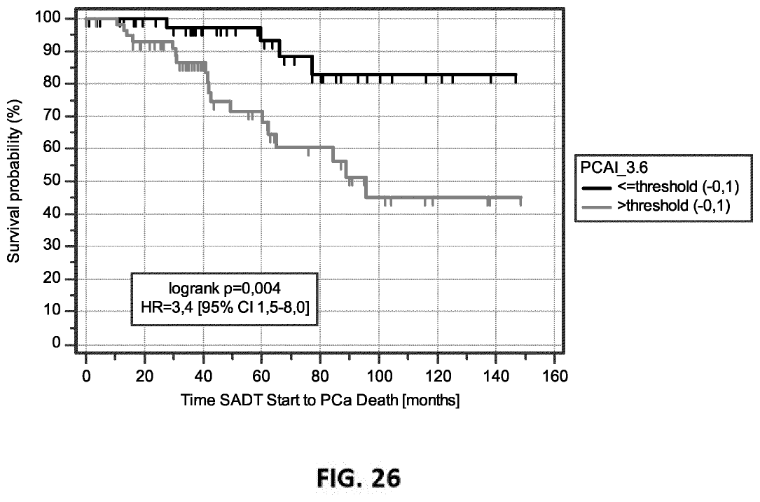

FIG. 26 shows a Kaplan-Meier curve for the PCAI-3.6 model. The clinical endpoint tested was PCa Death after start of SADT due to post-surgical disease recurrence. Subjects were stratified into two cohorts according to their risk to experience the clinical endpoint as predictend by PCAI-3.6 model (low vs. high risk). The value −0.1 was used as cut-off (log rank p=0.004; HR=3.4; CI=1.5-8.0). The following supplementary list indicate the number of subjects at risk for the analyzed PCAI-3.6 model classes, i.e. the subjects at risk at any time interval +20 months after surgery are shown: Low Risk (<=−0.1): 49, 39, 28, 22, 14, 7, 4, 1, 0; High risk (>−0.1): 57, 49, 29, 21, 14, 7, 3, 1, 0.

The Kaplan-Meier analyses as shown in the FIGS. 15-26 demonstrate that different subject/patient risk groups can also be distinguished using risk models that are based on a randomly selected combination of three genes, having per random combination one immune defense response gene, one TCR signaling gene and one PDE4D7 correlated gene and/or on a randomly selected combination of three genes, having per random combination two TCR signaling gene and one PDE4D7 correlated gene (FIGS. 17 & 23).

FIG. 27 shows a Kaplan-Meier curve for the PCAI-3.1 model. The clinical endpoint tested was PCa Death after surgery. Subjects were stratified into two cohorts according to their risk to experience the clinical endpoint as predictend by PCAI-3.1 model (low vs. high risk). The value 0 was used as cut-off (log rank p=0.0001; HR=4.3; CI=2.1-9.2). The following supplementary list indicate the number of subjects at risk for the analyzed PCAI-3.1 model classes, i.e. the subjects at risk at any time interval +20 months after surgery are shown: Low Risk (<=0): 301, 280, 266, 254, 230, 194, 160, 80, 16, 2, 0; High risk (>0): 270, 251, 233, 211, 189, 156, 129, 64, 8, 2, 0.

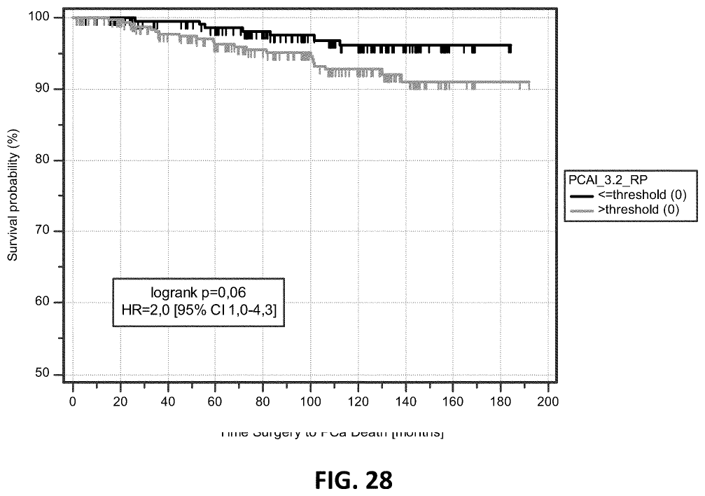

FIG. 28 shows a Kaplan-Meier curve for the PCAI-3.2 model. The clinical endpoint tested was PCa Death after surgery. Subjects were stratified into two cohorts according to their risk to experience the clinical endpoint as predictend by PCAI-3.2 model (low vs. high risk). The value 0 was used as cut-off (log rank p=0.06; HR=2.0; CI=1.0-4.3). The following supplementary list indicate the number of subjects at risk for the analyzed PCAI-3.2 model classes, i.e. the subjects at risk at any time interval +20 months after surgery are shown: Low Risk (<=0): 237, 221, 211, 200, 182, 147, 127, 61, 12, 2, 0; High risk (>0): 334, 310, 288, 265, 237, 203, 162, 83, 12, 2, 0.

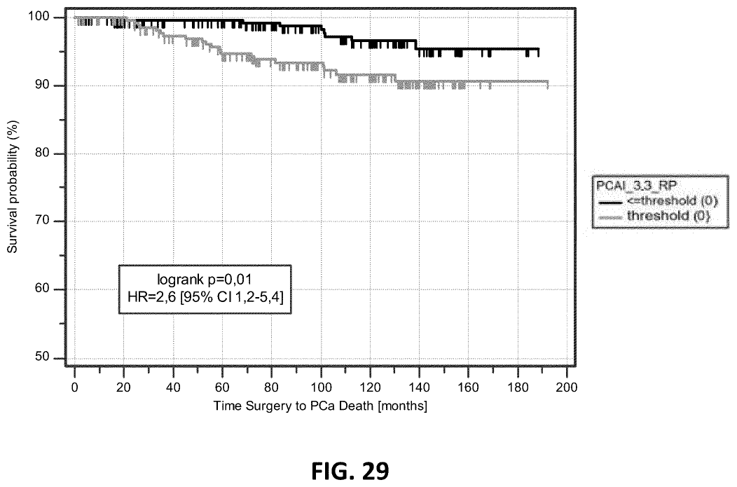

FIG. 29 comprises a reference figure showing a Kaplan-Meier curve for the PCAI-3.3 model. The clinical endpoint tested was PCa Death after surgery. Subjects were stratified into two cohorts according to their risk to experience the clinical endpoint as predictend by PCAI-3.3 model (low vs. high risk). The value 0 was used as cut-off (log rank p=0.01; HR=2.6; CI=1.2-5.4). The following supplementary list indicate the number of subjects at risk for the analyzed PCAI-3.3 model classes, i.e. the subjects at risk at any time interval +20 months after surgery are shown: Low Risk (<=0): 291, 268, 255, 243, 222, 189, 156, 77, 18, 3, 0; High risk (>0): 280, 263, 244, 222, 197, 161, 133, 67, 6, 1, 0.

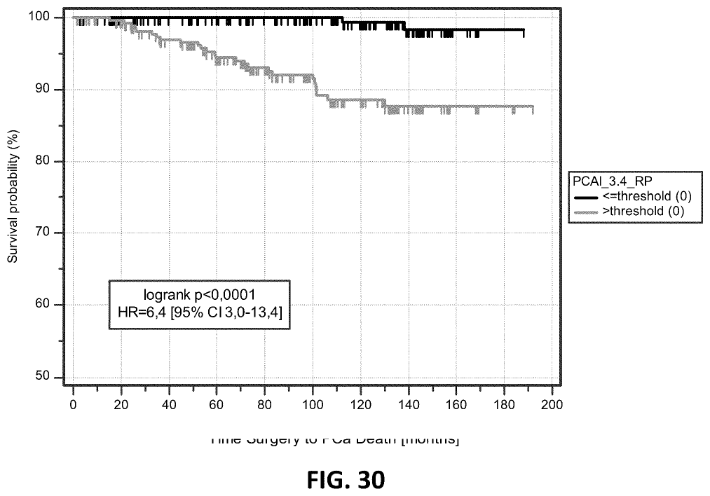

FIG. 30 shows a Kaplan-Meier curve for the PCAI-3.4 model. The clinical endpoint tested was PCa Death after surgery. Subjects were stratified into two cohorts according to their risk to experience the clinical endpoint as predictend by PCAI-3.4 model (low vs. high risk). The value 0 was used as cut-off (log rank p<0.0001; HR=6.4; CI=3.0-13.4). The following supplementary list indicate the number of subjects at risk for the analyzed PCAI-3.4 model classes, i.e. the subjects at risk at any time interval +20 months after surgery are shown: Low Risk (<=0): 292, 265, 255, 242, 228, 193, 164, 85, 13, 1, 0; High risk (>0): 279, 266, 244, 223, 191, 157, 125, 59, 11, 3, 0.

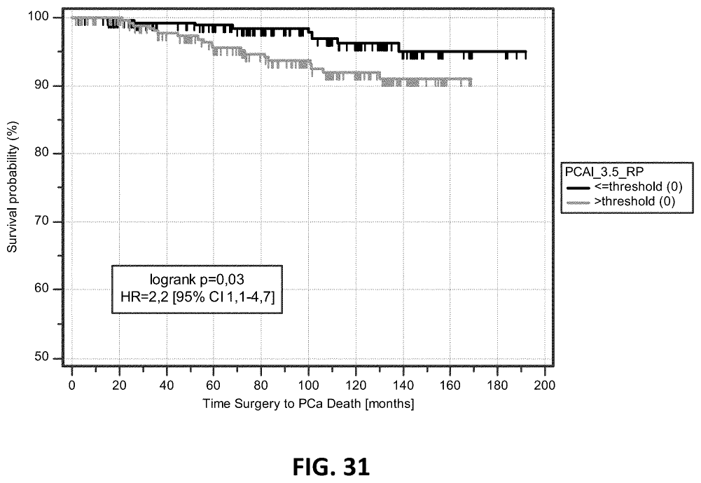

FIG. 31 shows a Kaplan-Meier curve for the PCAI-3.5 model. The clinical endpoint tested was PCa Death after surgery. Subjects were stratified into two cohorts according to their risk to experience the clinical endpoint as predictend by PCAI-3.5 model (low vs. high risk). The value 0 was used as cut-off (log rank p=0.03; HR=2.2; CI=1.1-4.7). The following supplementary list indicate the number of subjects at risk for the analyzed PCAI-3.5 model classes, i.e. the subjects at risk at any time interval +20 months after surgery are shown: Low Risk (<=0): 292, 268, 255, 243, 219, 187, 154, 74, 17, 4, 0; High risk (>0): 279, 263, 244, 222, 200, 163, 135, 70, 7, 0, 0.

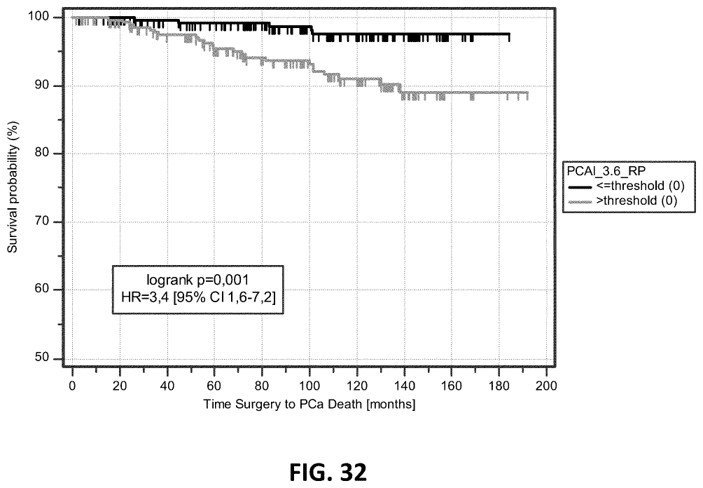

FIG. 32 shows a Kaplan-Meier curve for the PCAI-3.6 model. The clinical endpoint tested was PCa Death after surgery. Subjects were stratified into two cohorts according to their risk to experience the clinical endpoint as predictend by PCAI-3.6 model (low vs. high risk). The value 0 was used as cut-off (log rank p=0.001; HR=3.4; CI=1.6-7.2). The following supplementary list indicate the number of subjects at risk for the analyzed PCAI-3.6 model classes, i.e. the subjects at risk at any time interval +20 months after surgery are shown: Low Risk (<=0): 274, 257, 242, 230, 207, 168, 137, 77, 14, 1, 0; High risk (>0): 297, 274, 257, 235, 212, 182, 152, 67, 10, 3, 0.

The Kaplan-Meier analyses as shown in the FIGS. 27-32 demonstrate that different subject/patient risk groups tested on the endpoint of PCa Death after surgery comprises patients that have not been treated with SRT can also be distinguished using risk models that are based on a randomly selected combination of three genes, having per random combination one immune defense response gene, one TCR signaling gene and one PDE4D7 correlated gene and/or on a randomly selected combination of three genes, having per random combination two TCR signaling gene and one PDE4D7 correlated gene (FIG. 29).

### Discussion

The effectiveness of both radical RT and SRT for localized prostate cancer is limited, resulting in disease progression and ultimately death of patients, especially for those at high risk of recurrence. The prediction of the therapy outcome is very complicated as many factors play a role in therapy effectiveness and disease recurrence. It is likely that important factors have not yet been identified, while the effect of others cannot be determined precisely. Multiple clinico-pathological measures are currently investigated and applied in a clinical setting to improve response prediction and therapy selection, providing some degree of improvement. Nevertheless, a strong need remains for better prediction of the response to radical RT and to SRT, in order to increase the success rate of these therapies.

We have identified molecules of which expression shows a significant relation to mortality after radical RT and SRT and/or SADT and/or CTX and therefore are expected to improve the prediction of the effectiveness of these treatments. An improved prediction of effectiveness of RT for each patient be it in the radical or the salvage setting, will improve therapy selection and potentially survival. This can be achieved by 1) optimizing RT for those patients where RT is predicted to be effective (e.g. by dose escalation or a different starting time) and 2) guiding patients where RT is predicted not to be effective to an alternative, potentially more effective form of treatment. Further, this would reduce suffering for those patients who would be spared ineffective therapy and would reduce cost spent on ineffective therapies. Similar effects can be achieved for SADT and/or CTX.

Other variations to the disclosed realizations can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality.

One or more steps of the method illustrated in FIG. 1 may be implemented in a computer program product that may be executed on a computer. The computer program product may comprise a non-transitory computer-readable recording medium on which a control program is recorded (stored), such as a disk, hard drive, or the like. Common forms of non-transitory computer-readable media include, for example, floppy disks, flexible disks, hard disks, magnetic tape, or any other magnetic storage medium, CD-ROM, DVD, or any other optical medium, a RAM, a PROM, an EPROM, a FLASH-EPROM, or other memory chip or cartridge, or any other non-transitory medium from which a computer can read and use.

Alternatively, the one or more steps of the method may be implemented in transitory media, such as a transmittable carrier wave in which the control program is embodied as a data signal using transmission media, such as acoustic or light waves, such as those generated during radio wave and infrared data communications, and the like.

The exemplary method may be implemented on one or more general purpose computers, special purpose computer(s), a programmed microprocessor or microcontroller and peripheral integrated circuit elements, an ASIC or other integrated circuit, a digital signal processor, a hardwired electronic or logic circuit such as a discrete element circuit, a programmable logic device such as a PLD, PLA, FPGA, Graphical card CPU (GPU), or PAL, or the like. In general, any device, capable of implementing a finite state machine that is in turn capable of implementing the flowchart shown in FIG. 1, can be used to implement one or more steps of the method of risk stratification for therapy selection in a patient with prostate cancer is illustrated. As will be appreciated, while the steps of the method may all be computer implemented, in some embodiments one or more of the steps may be at least partially performed manually.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified herein.

Any reference signs in the claims should not be construed as limiting the scope.

The invention relates to a method of predicting a response of a prostate cancer subject to therapy or of personalizing therapy of a prostate cancer subject, comprising determining or receiving the result of a determination of a first gene expression profile for each of one or more, for example, 1, 2, 3, 4, 5, 6, 7, 8, 9, 10, 11, 12, 13 or all, immune defense response genes selected from the group consisting of: AIM2, APOBEC3A, CIAO1, DDX58, DHX9, IFI16, IFIH1, IFIT1, IFIT3, LRRFIP1, MYD88, OAS1, TLR8, and ZBP1, said first gene expression profile(s) being determined in a biological sample obtained from the subject, determining or receiving the result of a determination of a second gene expression profile for each of one or more, for example, 1, 2, 3, 4, 5, 6, 7, 8, 9, 10, 11, 12, 13, 14, 15, 16 or all, T-Cell receptor signaling genes selected from the group consisting of: CD2, CD247, CD28, CD3E, CD3G, CD4, CSK, EZR, FYN, LAT, LCK, PAG1, PDE4D, PRKACA, PRKACB, PTPRC, and ZAP70, said second gene expression profile(s) being determined in a biological sample obtained from the subject, determining or receiving the result of a determination of a third gene expression profile for each of one or more, for example, 1, 2, 3, 4, 5, 6, 7 or all, PDE4D7 correlated genes selected from the group consisting of: ABCC5, CUX2, KIAA1549, PDE4D, RAP1GAP2, SLC39A11, TDRD1, and VWA2, said third gene expression profile(s) being determined in a biological sample obtained from the subject, determining the prediction of the therapy response or the personalization of the therapy based on the first, second, and third gene expression profile(s), and, optionally, providing the prediction or the personalization or a therapy recommendation based on the prediction or the personalization to a medical caregiver or the subject. Since the status of the immune system and of the immune microenvironment have an impact on therapy effectiveness, the ability to identify markers predictive for this effect might help to be better able to predict response to pre-surgical RT as well as post-surgical therapies like SRT, SADT or CTX. The identified genes were found to exhibit a significant correlation with outcome after SRT and/or SADT and/or CTX. We therefore expect that they will provide predictive value with regard to the effectiveness of radical RT and/or post-surgical SRT and/or SADT and/or CTX.

The attached Sequence Listing, entitled 2020PF00492_Sequence Listing_ST25 is incorporated herein by reference, in its entirety.

1. A method of predicting a response of a prostate cancer subject to therapy or of personalizing therapy of a prostate cancer subject, comprising:
obtaining a first gene expression profile for each of one or more immune defense response genes selected from the group consisting of: AIM2, APOBEC3A, CIAO1, DDX58, DHX9, IFI16, IFIH1, IFIT1, IFIT3, LRRFIP1, MYD88, OAS1, TLR8, and ZBP1, said first gene expression profile(s) being determined in a biological sample obtained from the subject;
obtaining a second gene expression profile for each of one or more T-Cell receptor signaling genes selected from the group consisting of: CD2, CD247, CD28, CD3E, CD3G, CD4, CSK, EZR, FYN, LAT, LCK, PAG1, PDE4D, PRKACA, PRKACB, PTPRC, and ZAP70, said second gene expression profile(s) being determined in a biological sample obtained from the subject;
obtaining a third gene expression profile for each of one or more PDE4D7 correlated genes selected from the group consisting of: ABCC5, CUX2, KIAA1549, PDE4D, RAP1GAP2, SLC39A11, TDRD1, and VWA2, said third gene expression profile(s) being determined in a biological sample obtained from the subject;
determining at least one of: the prediction of the therapy response and/or the personalization of the therapy based on the first, second, and third gene expression profile(s); and
providing at least one of: the prediction and/or the personalization or a therapy recommendation based on the prediction or the personalization to a medical caregiver or the subject.

2. The method as defined in claim 1, wherein:
the one or more immune defense response genes comprise three or more of the immune defense genes, and/or
the one or more T-Cell receptor signaling genes comprise three or more of the T-Cell receptor signaling genes, and/or
the one or more PDE4D7 correlated genes comprise three or more of the PDE4D7 correlated genes.

3. The method as defined in claim 1, wherein the determining of the prediction of the therapy response or of the personalization of the therapy comprises:
combining the first gene expression profiles for two or more of the immune defense response genes with a regression function that had been derived from a population of prostate cancer subjects; and/or
combining the second gene expression profiles for two or more of the T-Cell receptor signaling genes with a regression function that had been derived from a population of prostate cancer subjects; and/or
combining the third gene expression profiles for two or more of the PDE4D7 correlated genes with a regression function that had been derived from a population of prostate cancer subjects.

4. The method as defined in claim 3, wherein the determining of the prediction of the therapy response or of the personalization of the therapy further comprises combining the combination of the first gene expression profiles, the combination of the second gene expression profiles, and the combination of the third gene expression profiles with a regression function that had been derived from a population of prostate cancer subjects.

5. The method as defined in claim 1, wherein the determining of the prediction of the therapy response or of the personalization of the therapy is further based on one or more clinical parameters obtained from the subject.

6. The method as defined in claim 4, wherein the clinical parameters comprise one or more of: (i) a prostate-specific antigen (PSA) level; (ii) a pathologic Gleason score (pGS); (iii) a clinical tumour stage; (iv) a pathological Gleason grade group (pGGG); (v) a pathological stage; (vi) one or more pathological variables; (vii) CAPRA; (viii) CAPRA-S; (ix) EAU-BCR risk groups; and (x) another clinical risk score.

7. The method as defined in claim 4, wherein the determining of the prediction of the therapy response or of the personalization of the therapy comprises combining one or more of: (i) the first gene expression profile(s) for the one or more immune defense response genes; (ii) the second gene expression profile(s) for the one or more T-Cell receptor signaling genes; (iii) the third gene expression profile(s) for the one or more PDE4D7 correlated genes, and; (iv) the combination of the first gene expression profiles, the combination of the second gene expression profiles, and the combination of the third gene expression profiles, and the one or more clinical parameters obtained from the subject with a regression function that had been derived from a population of prostate cancer subjects.

8. The method as defined in claim 1, wherein the biological sample(s) is/are obtained from the subject before the start of the therapy.

9. The method as defined in claim 1, wherein the therapy is radical radiotherapy, salvage radiotherapy (SRT), salvage androgen deprivation therapy (SADT), or cytotoxic chemotherapy (CTX).

10. The method as defined in claim 1, wherein the therapy is radiotherapy, wherein the prediction of the therapy response is negative or positive for the effectiveness of the therapy, wherein a therapy is recommended based on the prediction and, if the prediction is negative, the recommended therapy comprises one or more of: (i) radiotherapy provided earlier than is the standard; (ii) radiotherapy with an increased radiation dose; (iii) an adjuvant therapy, such as androgen deprivation therapy; and iv) an alternative therapy that is not a radiation therapy.

11. A non-transitory computer program product comprising instructions which, when the program is executed by a computer, cause the computer to carry out a method comprising:
receiving data indicative of a first gene expression profile for each of one or more immune defense response genes selected from the group consisting of: AIM2, APOBEC3A, CIAO1, DDX58, DHX9, IFI16, IFIH1, IFIT1, IFIT3, LRRFIP1, MYD88, OAS1, TLR8, and ZBP1, said first gene expression profile(s) being determined in a biological sample obtained from the subject;
receiving data indicative of a second gene expression profile for each of one or more T Cell receptor signaling genes selected from the group consisting of: CD2, CD247, CD28, CD3E, CD3G, CD4, CSK, EZR, FYN, LAT, LCK, PAG1, PDE4D, PRKACA, PRKACB, PTPRC, and ZAP70, said second gene expression profile(s) being determined in a biological sample obtained from the subject
receiving data indicative of a third gene expression profile for each of one or more PDE4D7 correlated genes selected from the group consisting of: ABCC5, CUX2, KIAA1549, PDE4D, RAP1GAP2, SLC39A11, TDRD1, and VWA2, said third gene expression profile(s) being determined in a biological sample obtained from the subject;
determining a prediction of a response of a prostate cancer subject to therapy or a personalization of therapy of a prostate cancer subject based on the first, second, and third gene expression profile(s); and
providing the prediction or the personalization or a therapy recommendation based on the prediction or the personalization to a medical caregiver or the subject.

12. An apparatus for predicting a response of a prostate cancer subject to therapy or for personalizing therapy of a prostate cancer subject, comprising:
an input adapted to receive: (i) data indicative of a first gene expression profile for each of one or more immune defense response genes selected from the group consisting of: AIM2, APOBEC3A, CIAO1, DDX58, DHX9, IFI16, IFIH1, IFIT1, IFIT3, LRRFIP1, MYD88, OAS1, TLR8, and ZBP1, said first gene expression profile(s) being determined in a biological sample obtained from the subject, (ii) data indicative of a second gene expression profile for each of one or more T Cell receptor signaling genes selected from the group consisting of: CD2, CD247, CD28, CD3E, CD3G, CD4, CSK, EZR, FYN, LAT, LCK, PAG1, PDE4D, PRKACA, PRKACB, PTPRC, and ZAP70, said second gene expression profile(s) being determined in a biological sample obtained from the subject, and (iii) data indicative of a third gene expression profile for each of one or more PDE4D7 correlated genes selected from the group consisting of: ABCC5, CUX2, KIAA1549, PDE4D, RAP1GAP2, SLC39A11, TDRD1, and VWA2, said third gene expression profile(s) being determined in a biological sample obtained from the subject;
a processor adapted to determine the prediction of the therapy response or the personalization of the therapy based on the first, second, and third gene expression profile(s); and
a non-transitory computer program product according to claim 11.

13. A diagnostic kit, comprising:
at least one primer and probe for determining a first gene expression profile for each of one or more immune defense response genes selected from the group consisting of: AIM2, APOBEC3A, CIAO1, DDX58, DHX9, IFI16, IFIH1, IFIT1, IFIT3, LRRFIP1, MYD88, OAS1, TLR8, and ZBP1, in a biological sample obtained from the subject;
at least one primer and probe for determining a second gene expression profile for each of one or more T Cell receptor signaling genes selected from the group consisting of: CD2, CD247, CD28, CD3E, CD3G, CD4, CSK, EZR, FYN, LAT, LCK, PAG1, PDE4D, PRKACA, PRKACB, PTPRC, and ZAP70, in a biological sample obtained from the subject; and
at least one primer and probe for determining a third gene expression profile for each of one or more PDE4D7 correlated genes selected from the group consisting of: ABCC5, CUX2, KIAA1549, PDE4D, RAP1GAP2, SLC39A11, TDRD1, and VWA2, in a biological sample obtained from the subject.

14. A method of using the kit as defined in claim 13 for determining at least one of: a first gene expression profile, a second gene expression profile, and/or a third gene expression profile.

15. The method of using the kid defined in claim 14 wherein the determined first gene expression profile, second gene expression profile, and/or third gene expression profile are used in predicting a response of a prostate cancer subject to therapy or of personalizing therapy of a prostate cancer subject.

16. A method, comprising:
receiving one or more biological sample(s) obtained from a prostate cancer subject,
using the kit as defined in claim 13 to determine a first gene expression profile for each of one or more immune defense response genes selected from the group consisting of: AIM2, APOBEC3A, CIAO1, DDX58, DHX9, IFI16, IFIH1, IFIT1, IFIT3, LRRFIP1, MYD88, OAS1, TLR8, and ZBP1, in a biological sample obtained from the subject,
using the kit as defined in claim 13 to determine a second gene expression profile for each of one or more T Cell receptor signaling genes selected from the group consisting of: CD2, CD247, CD28, CD3E, CD3G, CD4, CSK, EZR, FYN, LAT, LCK, PAG1, PDE4D, PRKACA, PRKACB, PTPRC, and ZAP70, in a biological sample obtained from the subject,
using the kit as defined in claim 13 to determine a third gene expression profile for each of one or more PDE4D7 correlated genes selected from the group consisting of: ABCC5, CUX2, KIAA1549, PDE4D, RAP1GAP2, SLC39A11, TDRD1, and VWA2, in a biological sample obtained from the subject.

17. The apparatus according to claim 12 wherein the non-transitory computer program product further:
determines the prediction of the therapy response or the personalization of the therapy based on the first, second, and third gene expression profile(s), and
provides the prediction or the personalization or a therapy recommendation based on the prediction or the personalization to a medical caregiver or the subject.

18. The method of claim 6, wherein the one or more pathological variables comprises at least one of: a status of surgical margins and/or a lymph node invasion and/or an extra-prostatic growth and/or a seminal vesicle invasion.
